(19) United States
(12) Patent Application Publication
(10) Pub. No.: US 2026/0066131 A1
(43) Pub. Date: Mar. 5, 2026

(54) METHODS FOR TREATING HEMOPHILIA A AND ESTIMATING BLEED RISK AND USES THEREOF

(71) Applicant: BIOVERATIV THERAPEUTICS INC., Waltham, MA (US)

(72) Inventors: Pratik BHAGUNDE, Cambridge, MA (US); Anaïs GLATARD, Uppsala (SE); Suresh KATRAGADDA, Cambridge, MA (US); Joakim NYBERG, Uppsala (SE)

(21) Appl. No.: 19/281,258

(22) Filed: Jul. 25, 2025

Related U.S. Application Data
(63) Continuation of application No. PCT/US2024/013782, filed on Jan. 31, 2024.
(60) Provisional application No. 63/482,631, filed on Feb. 1, 2023.
(60) Provisional application No. 63/485,658, filed on Feb. 17, 2023.
(60) Provisional application No. 63/592,100, filed on Oct. 20, 2023.

(51) Int. Cl. G16H 50/30

(52) U.S. Cl. CPC G16H 50/30

(57) ABSTRACT

The present disclosure provides methods for estimating bleed risk for subjects with hemophilia A, as well as computer-based-systems that comprise and implement repeated time to event (RTTE) models.

### CROSS-REFERENCE TO RELATED APPLICATIONS

This application is a continuation of U.S. Patent Application Ser. No. PCT/US2024/013782, filed Jan. 31, 2024, which claims the benefit of U.S. Provisional Patent Application Nos. 63/482,631 filed Feb. 1, 2023; 63/485,658 filed Feb. 17, 2023; and 63/592,100 filed Oct. 20, 2023; each of which is incorporated by reference in its entirety for all purposes.

### REFERENCE TO SEQUENCE LISTING SUBMITTED ELECTRONICALLY

The content of the electronically submitted sequence listing in XML file (Name: 765920_SA9-504PCCON_ST26.xml; Size: 33,373 bytes; and Date of Creation: Jul. 25, 2025) is incorporated herein by reference in its entirety.

### BACKGROUND

While plasma-derived and recombinant clotting factor products allow hemophilia patients to live longer and healthier lives, hemophilia remains one of the most costly and complex conditions to manage. Due to its complexity, treatment of hemophilia A using FVIII replacement therapy requires a special therapeutic management process for doctors, pharmacies, and patients. Clinicians often assess lifestyle, psychosocial requirements, and the home environment when evaluating a patient's or guardian's ability to provide adequate care.

The current recommended standard of care involves the regular administration (routine prophylaxis) of FVIII to minimize the number of bleeding episodes. Routine prophylaxis has been associated with improvements in long-term outcomes, but is a demanding regimen limited by the need for frequent intravenous (IV) administration. See Manco-Johnson et al., N Engl J Med. 357(6):535-44 (2007). Extended half-life FVIII products have reduced the frequency of FVIII administration for prophylaxis; however, most currently-available FVIII products interact with endogenous von Willebrand factor (VWF) and have comparable circulating half-lives, consistent with an upper limit on the half-life of rFVIII variants due to the half-life of endogenous VWF. See, e.g., Pipe et al., Blood. 128 (16): 2007-16 (2016).

### BRIEF SUMMARY

Disclosed herein are, inter alia, methods of assessing bleed risk for subjects with hemophilia A and computer-based systems comprising a repeated time to event (RTTE) model and their use to estimate bleed risk of subjects with hemophilia A. In some embodiments, the RTTE model comprises treatment effect as a covariate. In some embodiments, the treatment effect is prophylaxis treatment or on-demand treatment. In some embodiments, the RTTE model is the RTTE model [B]. In some embodiments, the RTTE model is the RTTE model [C]. In some embodiments, the RTTE model is the RTTE model [D]. In some embodiments, the RTTE model is the RTTE model [D1]. In some embodiments, the RTTE model is the RTTE model [D2]. In some embodiments, estimating bleed risk is estimating the risk of occurrence of a bleeding event.

Certain aspects of the present disclosure are directed to methods of estimating bleed risk comprising receiving clotting Factor VIII (FVIII) activity information by a software-based system comprising a computer program that is programmed to implement an RTTE model [B]; calculating, by the computer program, bleed risk using the RTTE model [B] and the received information, and transmitting, by the software-based system, the calculated bleed risk information of (b) for output of the bleed risk information.

Certain aspects of the present disclosure are directed to methods of estimating bleed risk comprising receiving clotting Factor VIII (FVIII) activity information by a software-based system comprising a computer program that is programmed to implement an RTTE model [B]; calculating, by the computer program, bleed risk using the RTTE model [C] and the received information, and transmitting, by the software-based system, the calculated bleed risk information of (b) for output of the bleed risk information.

Certain aspects of the present disclosure are directed to methods of estimating bleed risk comprising receiving clotting Factor VIII (FVIII) activity information by a software-based system comprising a computer program that is programmed to implement an RTTE model [B]; calculating, by the computer program, bleed risk using the RTTE model [D1] and the received information, and transmitting, by the software-based system, the calculated bleed risk information of (b) for output of the bleed risk information.

Certain aspects of the present disclosure are directed to methods of estimating bleed risk comprising receiving clotting Factor VIII (FVIII) activity information by a software-based system comprising a computer program that is programmed to implement an RTTE model [B]; calculating, by the computer program, bleed risk using the RTTE model [D2] and the received information, and transmitting, by the software-based system, the calculated bleed risk information of (b) for output of the bleed risk information.

Certain aspects of the present disclosure are directed to methods of estimating bleed risk comprising receiving, by one or more electronic devices, FVIII activity information; transmitting, by a processing device, the FVIII information to a software-based system, wherein the software-based system is programmed to implement an RTTE model [B] to calculate bleed risk; receiving, from the software-based system, bleed risk information calculated using the transmitted information of (b) and the RTTE model [B]; and transmitting, by the one or more electronic devices, the bleed risk information of (c) for output of the bleed risk information.

Certain aspects of the present disclosure are directed to methods of estimating bleed risk comprising receiving, by one or more electronic devices, FVIII activity information; transmitting, by a processing device, the FVIII information to a software-based system, wherein the software-based system is programmed to implement an RTTE model [C] to calculate bleed risk; receiving, from the software-based system, bleed risk information calculated using the transmitted information of (b) and the RTTE model [C]; and transmitting, by the one or more electronic devices, the bleed risk information of (c) for output of the bleed risk information.

Certain aspects of the present disclosure are directed to methods of estimating bleed risk comprising receiving, by one or more electronic devices, FVIII activity information; transmitting, by a processing device, the FVIII information to a software-based system, wherein the software-based system is programmed to implement an RTTE model [D1] to calculate bleed risk; receiving, from the software-based system, bleed risk information calculated using the transmitted information of (b) and the RTTE model [D1]; and transmitting, by the one or more electronic devices, the bleed risk information of (c) for output of the bleed risk information.

Certain aspects of the present disclosure are directed to methods of estimating bleed risk comprising receiving, by one or more electronic devices, FVIII activity information; transmitting, by a processing device, the FVIII information to a software-based system, wherein the software-based system is programmed to implement an RTTE model [D2] to calculate bleed risk; receiving, from the software-based system, bleed risk information calculated using the transmitted information of (b) and the RTTE model [D2]; and transmitting, by the one or more electronic devices, the bleed risk information of (c) for output of the bleed risk information.

Certain aspects of the present disclosure are directed to methods for estimating bleed risk for an individual subject, the method comprising receiving, by a software-based system, individualized subject information comprising the subject's body weight; receiving, by the software-based system, desired treatment outcome information comprising a FVIII activity level; applying an RTTE model for the subject based on the individualized information and/or the desired treatment outcome information; estimating bleed risk for the individual subject using the RTTE model; and transmitting, by the software-based system, the estimated bleed risk information of (d) for output of the bleed risk information. In some embodiments, the desired treatment outcome information is provided by the efanesoctocog alfa popPK model [A]. In some embodiments, the desired treatment outcome information is provided by the efanesoctocog alfa popPK model [A′]. In some embodiments, the desired treatment outcome information is provided by the individual subject. In some embodiments, the individual subject has severe hemophilia A. In some embodiments, the desired treatment outcome information is provided by a healthcare professional. In some embodiments, the RTTE model is RTTE model [B]. In some embodiments, the RTTE model is RTTE model [C]. In some embodiments, the RTTE model is RTTE model [D1]. In some embodiments, the RTTE model is RTTE model [D2].

Certain aspects of the present disclosure are directed to methods of treating a subject with hemophila A comprising identifying a subject with hemophilia A; estimating the bleed risk in the subject when treated with 50 IU/kg efanesoctocog alfa about once weekly; estimating the bleed risk in the subject if the subject had FVIII activity level of at least 10 IU/dL; and treating the subject with 50 IU/kg efanesoctocog alfa about once weekly if the subject has a lower estimated bleed risk with the efanesoctocog alfa therapy than the estimated bleed risk if the subject had a FVIII activity level of at least 10 IU/dL. In some embodiments, the estimating the bleed risk is calculated using a RTTE model. In some embodiments, the RTTE model is RTTE model [B]. In some embodiments, the RTTE model is RTTE model [C]. In some embodiments, the RTTE model is RTTE model [D1]. In some embodiments, the RTTE model is RTTE model [D2].

Certain aspects of the present disclosure are directed to methods for estimating bleed risk for an individual subject comprising (a) receiving information for an individual subject by a software-based system, wherein the system is programmed to implement (i) a one-compartment efanesoctocog alfa popPK model to calculate FVIII activity information, wherein the efanesoctocog alfa popPK model comprises body weight as a covariate, and wherein the efanesoctocog alfa popPK model does not comprise the level of VWF or level of hematocrit as covariates; and (ii) an RTTE model that uses the FVIII activity information to estimate bleed risk, (b) calculating, by the software-based system, estimated bleed risk using the efanesoctocog alfa popPK model, the RTTE model, and the received information, and (c) transmitting, by the software-based system, the calculated bleed risk information of (b) for output of the bleed risk information. In some embodiments, the RTTE model is RTTE model [B]. In some embodiments, the RTTE model is RTTE model [C]. In some embodiments, the RTTE model is RTTE model [D1]. In some embodiments, the RTTE model is RTTE model [D2]. In some embodiments, the individual subject has severe hemophilia A.

Certain aspects of the present disclosure are directed to methods of estimating bleed risk, the method comprising: (a) receiving, by one or more electronic devices, information for an individual subject; (b) transmitting, by a processing device, information for an individual subject to a software-based system, wherein the software-based system is programmed to implement (i) a one-compartment efanesoctocog alfa popPK model to calculate FVIII activity information, wherein the efanesoctocog alfa popPK model comprises body weight as a covariate, and wherein the efanesoctocog alfa popPK model does not comprise the level of VWF or level of hematocrit as covariates; and (ii) an RTTE model that uses the FVIII activity information to estimate bleed risk, (c) receiving, from the software-based system, estimated bleed risk using the efanesoctocog alfa popPK model, the RTTE model, and the received information; and (d) transmitting, by the one or more electronic devices, the bleed risk information of (c) for output of the bleed risk information. In some embodiments, the RTTE model is RTTE model [B]. In some embodiments, the RTTE model is RTTE model [C]. In some embodiments, the RTTE model is RTTE model [D1]. In some embodiments, the RTTE model is RTTE model [D2]. In some embodiments, the individual subject has severe hemophilia A.

In some embodiments, the FVIII activity information is provided by a popPK model. In some embodiments, the popPK model is an efanesoctocog alfa popPK model. In some embodiments, the efanesoctocog alfa popPK model comprises body weight as a covariate, and wherein the efanesoctocog alfa popPK model does not comprise the level of VWF or level of hematocrit as covariates. In some embodiments, the popPK model is the efanesoctocog alfa popPK model [A].

In some embodiments, the popPK model is the efanesoctocog alfa popPK model [A′].

In some embodiments, the FVIII activity information provided is for an individual subject.

In some embodiments, the subject has severe hemophilia A.

In some embodiments, the subject is less than 6 years old. In some embodiments, the subject is at least 6 years old. In some embodiments, the subject is less than 12 years old. In some embodiments, the subject is at least 12 years old. In some embodiments, the subject is at least 18 years old. In some embodiments, the subject is less than 18 years old.

In some embodiments, the FVIII activity information provided is the estimated FVIII activity level of a population of subjects. In some embodiments, the FVIII activity information provided is for a virtual or hypothetical subject. In some embodiments, the FVIII activity information provided is a target FVIII activity level. In some embodiments, the FVIII activity information provided is a variable FVIII activity level over time. In some embodiments, the subject had or has a FVIII activity level of at least 10 IU/dL.

Certain aspects of the present disclosure are directed to a data processing apparatus, device, or system comprising a processor configured to implement efanesoctocog alfa RTTE model [B]. In some embodiments, the RTTE model is RTTE model [C]. In some embodiments, the RTTE model is RTTE model [D1]. In some embodiments, the RTTE model is RTTE model [D2]. In some embodiments, the data processing apparatus, device, or system is further configured to implement a one-compartment efanesoctocog alfa popPK model that comprises body weight as a covariate, wherein the efanesoctocog alfa popPK model does not comprise the level of VWF or level of hematocrit as covariates. In some embodiments, the popPK model is efanesoctocog alfa popPK model [A]. In some embodiments, the popPK model is the efanesoctocog alfa popPK model [A′]. In some embodiments, the data processing apparatus, device, or system comprises a smart phone, a tablet computer, a personal digital assistant, a handheld computer, a laptop computer, or a smart watch. In some embodiments, the data processing apparatus, device, or system comprises a smart phone.

Certain aspects of the present disclosure are directed to a computer program comprising instructions which, when the program is executed by a computer, cause the computer to implement the efanesoctocog alfa RTTE model [B]. In some embodiments, the RTTE model is RTTE model [C]. In some embodiments, the RTTE model is RTTE model [D1]. In some embodiments, the RTTE model is RTTE model [D2]. In some embodiments, the RTTE model uses a PK profile (e.g., a level of FVIII activity over time) generated from a popPK model as an input. In some embodiments, the popPK model is an efanesoctocog alfa popPK model. In some embodiments, the efanesoctocog alfa popPK model is a one-compartment efanesoctocog alfa popPK model that comprises body weight as a covariate, wherein the efanesoctocog alfa popPK model does not comprise the level of VWF or level of hematocrit as covariates. In some embodiments, the popPK model is efanesoctocog alfa popPK model [A]. In some embodiments, the popPK model is the efanesoctocog alfa popPK model [A].

Certain aspects of the present disclosure are directed to a computer-readable medium comprising instructions which, when executed by a computer, cause the computer to carry out any of the methods disclosed herein.

Certain aspects of the present disclosure are directed to methods of treating hemophilia A in a human subject in need thereof, comprising intravenously administering to the subject from about 25 IU/kg to about 50 IU/kg of efanesoctocog alfa every about 4 to about 14 days, wherein the human subject is less than 6 years old and weighs from 15 to 20 kg.

Certain aspects of the present disclosure are directed to methods of treating hemophilia A in a human subject in need thereof, comprising intravenously administering to the subject from about 25 IU/kg to about 50 IU/kg of efanesoctocog alfa every about 4 to about 14 days, wherein the human subject is 6 to <12 years of age and weighs from 30 to 35 kg.

In some embodiments, the efanesoctocog alfa is administered at a dose of about 25 IU/kg every about 4 days. In some embodiments, the efanesoctocog alfa is administered at a dose of about 30 IU/kg every about 7 days. In some embodiments, the efanesoctocog alfa is administered at a dose of about 50 IU/kg every about 7 days. In some embodiments, the efanesoctocog alfa is administered at a dose of about 50 IU/kg every about 14 days. In some embodiments, the efanesoctocog alfa is administered at a dose of about 25 IU/kg every about 4 days for at least about 52 weeks. In some embodiments, the efanesoctocog alfa is administered at a dose of about 30 IU/kg every about 7 days for at least about 52 weeks. In some embodiments, the efanesoctocog alfa is administered at a dose of about 50 IU/kg every about 7 days for at least about 52 weeks. In some embodiments, the efanesoctocog alfa is administered at a dose of about 50 IU/kg every about 14 days for at least about 52 weeks.

Certain aspects of the present disclosure are directed to a pharmaceutical composition comprising efanesoctocog alfa for use in treating hemophilia A in a human subject in need thereof, wherein from about 25 IU/kg to about 50 IU/kg of efanesoctocog alfa is intravenously administered to the subject every about 4 to about 14 days during the period, and wherein the human subject is less than 6 years old and weighs from 15 to 20 kg.

Certain aspects of the present disclosure are directed to a pharmaceutical composition comprising efanesoctocog alfa for use in treating hemophilia A in a human subject in need thereof, wherein from about 25 IU/kg to about 50 IU/kg of efanesoctocog alfa is intravenously administered to the subject every about 4 to about 14 days during the period, and wherein the human subject is 6 to <12 years of age and weighs from 30 to 35 kg.

In some embodiments, the efanesoctocog alfa is administered at a dose of about 25 IU/kg every about 4 days. In some embodiments, the efanesoctocog alfa is administered at a dose of about 30 IU/kg every about 7 days. In some embodiments, wherein the efanesoctocog alfa is administered at a dose of about 50 IU/kg every about 7 days. In some embodiments, the efanesoctocog alfa is administered at a dose of about 50 IU/kg every about 14 days. In some embodiments, the efanesoctocog alfa is administered at a dose of about 25 IU/kg every about 4 days for at least about 52 weeks. In some embodiments, the efanesoctocog alfa is administered at a dose of about 30 IU/kg every about 7 days for at least about 52 weeks. In some embodiments, the efanesoctocog alfa is administered at a dose of about 50 IU/kg every about 7 days for at least about 52 weeks. In some embodiments, the efanesoctocog alfa is administered at a dose of about 50 IU/kg every about 14 days for at least about 52 weeks.

Certain aspects of the present disclosure are directed to methods of reducing the risk that a human subject with severe hemophilia A in need thereof will have a bleed that is not caused by trauma over a period of 52 weeks, wherein the risk is reduced to a probability of less than 50%, the method comprising intravenously administering to the subject from about 25 IU/kg to about 50 IU/kg of efanesoctocog alfa every about 4 to about 14 days during the period, wherein the human subject is 6 to <12 years of age and weighs from 30 to 35 kg, thereby reducing the probability that to less than 50%.

Certain aspects of the present disclosure are directed to methods of reducing the probability that a human subject with severe hemophilia A in need thereof will have a bleed that is not caused by trauma within the next 52 weeks, wherein the risk is reduced to a probability of less than 50%, the method comprising intravenously administering to the subject from about 25 IU/kg to about 50 IU/kg of efanesoctocog alfa every about 4 to about 14 days during the period, wherein the human subject is 6 to <12 years of age and weighs from 30 to 35 kg, thereby reducing the probability to less than 50%.

In some embodiments, the risk is reduced to a probability of less than 40%. In some embodiments, the risk is reduced to a probability of less than 30%. In some embodiments, the bleed that is not caused by trauma is a spontaneous bleed. As used herein, a “spontaneous bleed” is a bleed that occurs when there is no known contributing factor, such as a definite trauma or antecedent strenuous activity. In some embodiments, the efanesoctocog alfa is administered at a dose of about 25 IU/kg every about 4 days during the period. In some embodiments, the efanesoctocog alfa is administered at a dose of about 30 IU/kg every about 7 days during the period. In some embodiments, the efanesoctocog alfa is administered at a dose of about 50 IU/kg every about 7 days during the period. In some embodiments, the efanesoctocog alfa is administered at a dose of about 50 IU/kg every about 14 days during the period.

Certain aspects of the present disclosure are directed to pharmaceutical compositions comprising efanesoctocog alfa for use in reducing the risk that a human subject with severe hemophilia A in need thereof will have a bleed that is not caused by trauma over a period of 52 weeks, wherein the risk is reduced to a probability of less than 50%, wherein from about 25 IU/kg to about 50 IU/kg of efanesoctocog alfa is intravenously administered to the subject every about 4 to about 14 days during the period, wherein the human subject is 6 to <12 years of age, and wherein the subject weighs from 30 to 35 kg intravenously administering to the subject, thereby reducing the probability that to less than 50%.

Certain aspects of the present disclosure are directed to pharmaceutical compositions comprising efanesoctocog alfa for use in reducing the risk that a human subject with severe hemophilia A in need thereof will have a bleed that is not caused by trauma over a period of 52 weeks, wherein the risk is reduced to a probability of less than 50%, wherein from about 25 IU/kg to about 50 IU/kg of efanesoctocog alfa is intravenously administered to the subject every about 4 to about 14 days during the period, wherein the human subject is 6 to <12 years of age, and wherein the subject weighs from 30 to 35 kg, thereby reducing the probability to less than 50%.

In some embodiments, the the risk is reduced to a probability of less than 40%. In some embodiments, the risk is reduced to a probability of less than 30%. In some embodiments, the bleed that is not caused by trauma is a spontaneous bleed. In some embodiments, the efanesoctocog alfa is administered at a dose of about 25 IU/kg every about 4 days during the period. In some embodiments, the efanesoctocog alfa is administered at a dose of about 30 IU/kg every about 7 days during the period. In some embodiments, the efanesoctocog alfa is administered at a dose of about 50 IU/kg every about 7 days during the period. In some embodiments, the efanesoctocog alfa is administered at a dose of about 50 IU/kg every about 14 days during the period.

### BRIEF DESCRIPTION OF DRAWINGS/FIGURES

FIG. 1 is a visual representation of a software-based system that can be used in methods disclosed herein.

FIG. 2 is a visual representation of an exemplary network-based system that can be used according to the methods disclosed herein.

FIG. 3 shows a schematic diagram of an example computing system 400.

FIG. 4 is a graph showing simulated steady-state FVIII activity over time using efanesoctocog alfa population PK model [A] (50 IU/kg) in patients ≥12 years of age, based on clinical data derived from the one-stage clotting assay. Observed FVIII activity from the clinical data is also shown. Solid line is simulated median FVIII activity (IU/dL). Dashed lines are simulated 5th and 95th percentiles.

FIG. 5A is a graph showing a baseline corrected FVIII activity time profile from adult/adolescent study XTEND-1 (EFC16293, NCT04161495), at day 1. Data is shown for all patients. LLOQ=1 IU/dL. FIG. 5B is a graph showing a baseline corrected FVIII activity time profile from adult/adolescent study XTEND-1, at day 1 (solid line) and week 26 (dashed line). Data is shown for sequential arm patients only.

FIG. 6 illustrates correlation between 4 continuous covariates at baseline. WTKGB is baseline weight (median is 78.3, excluding EFC16295). BH is baseline race (median is 43, excluding EFC16295). BVWF is baseline VWF (median is 112, excluding EFC16295).

FIGS. 7A and 7B are graphs demonstrating population predictions (PRED) (FIG. 7A) and individual predictions (IPRED) (FIG. 7B) versus or observed value of one-stage FVIII activity (DV; data value) using efanesoctocog alfa population PK model [A]. Black line is line of unity. Grey line is loess smoothing line. R²=0.92 for the population predictions in FIG. 7A. R²=0.97 for the individual predictions in FIG. 7B.

FIG. 8 is a set of graphs demonstrating the performance of efanesoctocog alfa population PK model [A] using visual predictive checks (VPC). Open circles show observed data. Solid line shows the model simulated median. Dashed lines show model simulated 5^{th} and 95^{th} percentiles. Shading around each dashed line represents 90% CI around the simulated 5^{th} and 95^{th} percentiles. Shading around each solid line shows the 90% CI around the simulated median.

FIGS. 9A and 9B demonstrate PRED (FIG. 9A) and IPRED (FIG. 9B) versus DV for surgery using efanesoctocog alfa population PK model [A]. The grey line is the line of unity. R² is shown as a black line which is the regression line between observed and predicted.

FIG. 10 is a set of graphs showing the distribution of steady state C_{trough}, Cₘₐₓₛₛ and time to 40 IU/dL FVIII activity levels across body weight.

FIG. 11 is a set of graphs showing the distribution of steady state C_{trough}, Cₘₐₓₛₛ and time to 40 IU/dL FVIII activity levels across different age groups of non-Asian and Asian populations.

FIG. 12 is a graph showing OSC FVIII activity over time for major surgery and major bleeds for patients aged 6 and younger. Grey solid line: simulated median for 30 IU/kg dose. Grey line with circles: simulated median for 50 IU/kg dose. Black solid lines: simulated 5th and 95th percentiles. Dashed lines indicate OSC activity of 80 IU/kg and 40 IU/kg.

FIG. 13 is a graph showing simulated FVIII activity for a dose of 50 IU/kg efanesoctocog alfa followed by 30 IU/kg every 3 days until Day 14 in a virtual adult and adolescent population.

FIG. 14 is a graph showing simulated OSC FVIII activity over time for all age groups.

FIG. 15 is a graph showing Kaplan-Meier plots for patients without bleeding events for Arm A and Arm B in the XTEND-1 study.

FIG. 16 is a set of graphs showing Kaplan-Meier visual predictive checks of the final RTTE model for the prophylaxis arm in the XTEND-1 study.

FIG. 17 is a graph demonstrating the probability of a bleeding event in 1 year in a typical patient as generated by the RTTE model.

FIG. 18 is a set of histograms showing the distribution of covariates at baseline for the patients in the pediatric BIVV001 RTTE analysis data set. The median is shown as the vertical solid line on the left, and the mean is shown as the vertical dotted line on the right in each graph.

FIG. 19 is a correlation matrix of covariates at baseline for the patients in the pediatric BIVV001 RTTE analysis data set. For this plot, all covariates (continuous and categorical) are treated as continuous. Correlation coefficients, presented in the lower left side of the matrix, are also visualized as ellipses in the upper right side, with the ellipse's width corresponding to the absolute value of the correlation coefficient and the ellipse's slope indicating a positive or negative correlation. The three ellipses representing negative values are further identified by a darkened border. Correlation between each pair of variables is computed using all individuals with observations of both variables.

FIG. 20 is a set of graphs showing Kaplan-Meier visual predictive checks of the final pediatric RTTE model as applied to the pediatric BIVV001 RTTE dataset (n=74). The solid and dashed lines represent the Kaplan-Meier point estimate and 95% confidence interval for the observed data, respectively; the shaded areas represent the 95% confidence interval of the Kaplan-Meier point estimate based on 400 replicate simulations.

FIG. 21 is a set of graphs showing the predicted versus observed FVIII activity for EFC16295 and LTS16294 studies. The line with triangles is the line of unity while the dotted line is the loess smoothing line.

FIG. 22 is a set of graphs showing conditional weighted residuals (CWRES) vs time and CWRES vs PRED. The gray line is loess smoothing line.

FIG. 23 is a set of graphs showing individual weighted residuals (IWRES) vs time and IWRES vs IPRED. The gray line is loess smoothing line.

FIG. 24 is a graph showing visual predictive checks (VPC) of the final popPK model for the EFC16295 study.

FIG. 25 is a set of graphs showing Predicted vs Observed FVIII activity during and after surgery (post-op follow up). The line with triangles is the line of unity while the dotted line is the loess smoothing line.

FIG. 26 is a graph showing steady state AUCtau, Cmax and Cmin with baseline VWF for assessment of intrinsic/extrinsic factors. The gray line is loess smoothing line. Empty circles represent the group of children over one year of age, but under 6 years of age. Filled circles represent the group of children from 6 years of age and older, but under 12.

FIG. 27 is a graph showing steady state AUCtau, Cmax and Cmin with baseline body weight for assessment of intrinsic/extrinsic factors. The gray line is loess smoothing line. Empty circles represent the group of children over one year of age, but under 6 years of age. Filled circles represent the group of children from 6 years of age and older, but under 12.

FIG. 28 is a graph showing steady state AUCtau, Cmax and Cmin with baseline hematocrit for assessment of intrinsic/extrinsic factors. The gray line is loess smoothing line. Empty circles represent the group of children over one year of age, but under 6 years of age. Filled circles represent the group of children from 6 years of age and older, but under 12.

### DETAILED DESCRIPTION

With the emergence of extended half-life replacement products, treatment goals are now expanding beyond targeting a low annualized bleed rate (ABR) to include long-term outcomes associated with high sustained plasma FVIII activity levels, such as long-term joint protection. Efanesoctocog alfa circulates independently of endogenous von Willebrand factor (VWF), and provides high sustained FVIII activity (see, e.g., Chhabra, et al. Blood. 2020; 135(17):1484-1496, Konkle et al., N Engl J Med 2020; 383:1018-1027, and von Drygalski et al., N Engl J Med 2023; 388:310-318 (referring to efanesoctocog alfa as BIVV001), the entire contents of each of which are incorporated herein by reference for all purposes).

The present disclosure provides, inter alia, methods of treatment, software-based systems for estimating individualized subject efanesoctocog alfa PK information. In some embodiments, the software-based systems apply an efanesoctocog alfa population PK model [A] for estimating dose information for subjects receiving efanesoctocog alfa as FVIII replacement treatment.

The present disclosure also provides, inter alia, methods of treatment and software-based systems for estimating or quantifying the risk of a bleed associated with different levels of FVIII activity e.g., over time. In some embodiments, the software-based system can use an efanesoctocog alfa population pharmacokinetic model when quantifying the risk of bleed with high sustained FVIII activity compared to standard of care.

### Definitions

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

It is understood that wherever aspects are described herein with the language “comprising,” otherwise analogous aspects described in terms of “consisting of” and/or “consisting essentially of” are also provided.

As used herein in the context of hemophilia A, the term “prophylactic treatment” refers to the preemptive administration of a therapy for the treatment of hemophilia A, where such treatment is intended to prevent or reduce the severity of one or more symptoms of hemophilia A, e.g., bleeding episodes, such as one or more spontaneous bleeding episodes, and/or joint damage. To prevent or reduce the severity of such symptoms, e.g., bleeding episodes and the progression of joint disease, hemophilia A patients may receive regular infusions of clotting factor (such as efanesoctocog alfa) as part of a prophylactic treatment regimen.

The term “on-demand treatment” or “episodic treatment” refers to the “as needed” administration of a FVIII replacement therapy (such as efanesoctocog alfa) in response to symptoms of hemophilia A, e.g., a bleeding episode (such as a spontaneous bleeding episode or a traumatic bleeding episode), or before an activity that can cause bleeding. In some embodiments, the on-demand treatment can be given to a subject when bleeding starts, such as after an injury, or when bleeding is expected, such as before surgery. In some embodiments, the on-demand treatment can be given prior to activities that increase the risk of bleeding, such as contact sports. In some embodiments, on-demand treatment can be administered to a subject who is receiving prophylactic treatment, e.g., if supplemental FVIII replacement protein doses are administered to treat a bleeding episode or before strenuous activity. In some embodiments, the on-demand treatment is given as a single dose. In some embodiments, the on-demand treatment is given as a first dose, followed by one or more additional doses. In some embodiments, the on-demand regimen is for perioperative management of bleeding.

In some embodiments, a bleeding episode starts from the first sign of a bleed and ends 72 hours after the last treatment for the bleeding, within which any symptoms of bleeding at the same location, or injections less than or equal to 72 hours apart, is considered the same bleeding episode. See Blanchette V. (2006) Haemophilia 12:124-7. In some embodiments, any injection to treat the bleeding episode, taken more than 72 hours after the preceding one, is considered the first injection to treat a new bleeding episode at the same location. In some embodiments, any bleeding at a different location is considered a separate bleeding episode regardless of time from the last injection.

The methods provided herein can be applied to a subject in need of prophylactic treatment or episodic/on-demand treatment. In some embodiments, the subject in need of prophylactic treatment or episodic/on-demand treatment suffers from hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the iliopsoas sheath. In some embodiments, the subject is in need of treatment for surgery, including, e.g., surgical prophylaxis or peri-operative management. In some embodiments, the surgery is minor surgery or major surgery. Exemplary surgical procedures include tooth extraction, tonsillectomy, inguinal herniotomy, synovectomy, craniotomy, osteosynthesis, trauma surgery, intracranial surgery, intra-abdominal surgery, intrathoracic surgery, joint replacement surgery (e.g., total knee replacement, hip replacement, and the like), heart surgery, and caesarean section.

“Treat” and “treating”, as used herein in the context of hemophilia A include, e.g., the reduction in severity of hemophilia A; the amelioration of one or more symptoms associated with hemophilia A; the provision of beneficial effects to a subject with hemophilia A, without necessarily curing the hemophilia A; and/or prophylaxis for one or more symptoms associated with hemophilia A.

In some embodiments, treating hemophilia A includes prevention of one or more symptoms of hemophilia A (such as spontaneous bleeding). In some embodiments, treating hemophilia A includes reducing the likelihood of a bleeding episode/event, or reducing the severity of a bleeding episode/event. In some embodiments, treatment is prophylactic treatment. In some embodiments, treatment is on-demand treatment. In some embodiments, treating comprises the reduction of the frequency of one or more symptoms of hemophilia A, e.g., spontaneous or uncontrollable bleeding episodes.

The term “perioperative management” as used herein means use of efanesoctocog alfa before, concurrently with, or after an operative procedure, e.g., a surgical operation. The use for “perioperative management” of one or more bleeding episode includes surgical prophylaxis before (i.e., preoperative), during (i.e., intraoperative), or after (i.e., postoperative) a surgery to prevent one or more bleeding or bleeding episode or reducing or inhibiting spontaneous and/or uncontrollable bleeding episodes before, during, and after a surgery.

As used herein, a “baseline” plasma FVIII activity level is the lowest measured plasma FVIII activity level in a subject prior to administering a dose. In some embodiments, activity above the baseline pre-dosing can be considered residue FVIII activity from prior treatment, and can be decayed with time using the half-life of prior treatment and subtracted from the PK data following efanesoctocog alfa dosing.

The term “patient” and “subject” are used interchangeably herein and refer to a human. A subject may include, e.g., an individual who has been diagnosed with hemophilia A, and who is susceptible to spontaneous and/or uncontrolled bleeding episodes. Subjects can also include individuals who are in danger of one or more uncontrollable bleeding episodes prior to a certain activity, e.g., a surgery, a sport activity, or any strenuous activity. In some embodiments, the subject has a baseline FVIII activity less than 0.5%, less than 1%, less than 2%, less than 2.5%, less than 3%, or less than 4%. In some embodiments, the subject has severe hemophilia A, defined as <1 IU/dL (<1%) endogenous FVIII activity. In some embodiments, the subject has no coagulation disorder other than hemophilia A.

As used herein, the terms “ELNN polypeptides” and “ELNNs” are synonymous and refer to extended length polypeptides comprising non-naturally occurring, substantially non-repetitive sequences (e.g., polypeptide motifs) that are composed mainly of small hydrophilic amino acids, with the sequence having a low degree or no secondary or tertiary structure under physiologic conditions. ELNN polypeptides include unstructured hydrophilic polypeptides comprising repeating motifs of 6 natural amino acids (G, A, P, E, S, and/or T). In some embodiments, an ELNN polypeptide comprises multiple motifs of 6 natural amino acids (G, A, P, E, S, T), wherein the motifs are the same or comprise a combination of different motifs. In some embodiments, ELNN polypeptides can confer certain desirable pharmacokinetic, physicochemical, and pharmaceutical properties when linked to proteins, including T-cell engagers as disclosed herein. Such desirable properties may include but are not limited to enhanced pharmacokinetic parameters and solubility characteristics, as well as improved therapeutic index. ELNN polypeptides are known in the art, and non-limiting descriptions relating to and examples of ELNN polypeptides known as XTEN polypeptides are available in Schellenberger et al., (2009) Nat Biotechnol 27(12):1186-90; Brandl et al., (2020) Journal of Controlled Release 327:186-197; and Radon et al., (2021) Advanced Functional Materials 31, 2101633 (pages 1-33), the entire contents of each of which are incorporated herein by reference.

As used herein “software-based system” refers to an algorithm or set of algorithms capable of being implemented by one or more processing devices. The software-based system may be embodied in software which includes but is not limited to firmware, resident software, microcode, etc. and may take the form of one or more computer program products accessible from one or more computer-usable or computer-readable medium or media providing program code for use by or in connection with a computer or any instruction execution system. The software may be implemented across several processing devices that collectively comprise the software-based system. For the purposes of this description, a computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk, and an optical disk, including compact disc-read only memory (CD-ROM), compact disc-read/write (CD-R/N) and DVD. Non-limiting examples of software-based systems include network-based systems and web-based systems.

As used herein the term “processing device” refers to a data processing system suitable for storing and/or executing program code to implement the software-based system and may include at least one processor coupled directly or indirectly to memory elements through a system bus or other interface. The processor(s), i.e., the electronic circuitry that executes instructions that make up the program code, may be instantiated by a microprocessor, microcontroller, multi-core processor, array of processors, or vector processors, and may be embodied in one or across several physical devices. The memory elements can include local memory employed during actual execution of the program code, bulk storage, cloud storage, and cache memories which provide temporary storage of at least some program code in order to reduce the number of times code must be retrieved from bulk storage during execution. Input/output or I/O devices (including but not limited to keyboards, displays, pointing devices, touch screens, audio, etc.) can be coupled to the system either directly or through intervening I/O controllers. Network adapters may also be coupled to the system to enable the processing device to become coupled to other processing devices or remote printers or storage devices through intervening private or public networks. Modems, cable modems and Ethernet cards are just a few of the currently available types of network adapters. The processing device may also be a shared data processing system such as a network-based (e.g., web-based) server system, accessible via a network such as the Internet, that is capable of accessing and executing program code to implement the software-based system.

### Description of Efanesoctocog Alfa

Efanesoctocog alfa is described in Chhabra et al. Blood 2020; 135(17): 1484-1496, Konkle et al., N Engl J Med 2020; 383:1018-1027, von Drygalski et al., N Engl J Med 2023; 388:310-318, and the International Nonproprietary Names for Pharmaceutical Substances (INN) WHO Drug Information, 2019, Vol. 33, No. 4, p. 828-30, the entire contents of each of which are hereby incorporated by reference in their entireties. Efanesoctocog alfa temporarily replaces the missing FVIII needed for effective hemostasis in patients with a deficiency of FVIII. Efanesoctocog alfa comprises a first polypeptide comprising the amino acid sequence of SEQ ID NO: 3 covalently bound to a second polypeptide comprising the amino acid sequence of SEQ ID NO: 6, wherein the first polypeptide and the second polypeptide are covalently bound to each other via disulfide bonds. Efanesoctocog alfa may be produced, e.g., by recombinant DNA technology in a human embryonic kidney (HEK) cell line. For example, the cell line can express an rFVIIIFc-ELNN polypeptide (SEQ ID NO: 1), an rVWF-ELNN-Fc polypeptide (SEQ ID NO: 4), and a soluble PACE enzyme. Non-limiting examples of nucleotide sequences encoding the rFVIIIFc-ELNN polypeptide (SEQ ID NO: 2) and the rVWF-ELNN-Fc polypeptide polypeptide (SEQ ID NO: 5) can be found in Table A, below. Amino acid sequences for the rFVIIIFc-ELNN polypeptide without a signal peptide (SEQ ID NO: 3) and rVWF-ELNN-Fc polypeptide polypeptide without a signal peptide or D1D2 portion of VWF (SEQ ID NO: 6) can be found in Table A, below.

In some embodiments for subjects receiving prophylactic treatment with efanesoctocog alfa, the methods disclosed herein can be used to determine individualized subject information (e.g., the subject's plasma FVIII levels at a particular time point or a set of timepoints). Based on this individual subject information, the dose and/or dose interval of efanesoctocog alfa can be adjusted to achieve individual treatment goals, such as a minimum plasma FVIII activity level (e.g., trough).

In some embodiments, for subjects receiving on-demand treatment with efanesoctocog alfa, if the subject's bleeding is not controlled or is unsatisfactory after efanesoctocog alfa administration at an initial recommended dose and dose interval, the methods disclosed herein can be used to determine individual subject dose information. Based on this individual subject dose information, the dose and/or dose interval of efanesoctocog alfa can be adjusted to achieve improved bleed control. In some embodiments, the dose and/or dose interval is adjusted to achieve the desired estimated bleed risk.

In some embodiments, the methods disclosed herein can be used to estimate a minimum FVIII level between doses for a subject.

In some embodiments, a subject's plasma can be monitored for FVIII activity levels, e.g., the one-stage clotting assay, to confirm adequate FVIII levels have been achieved and maintained, when clinically indicated. FVIII activity can be measured by any known methods in the art.

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII. It is typically used in conjunction with prothrombin time (PT), which measures the extrinsic pathway. (See, e.g., Kamal et al., Mayo Clin Proc., 82(7):864-873 (2007)). In some embodiments, the aPTT assay uses Actin FSL as a reagent. In some embodiments, the aPTT assay does not use Actin FS as a reagent. In some embodiments, aPTT is tested using an assay where FVIII activity is measured using the Dade® Actin® FSL Activated PTT Reagent (Siemens Health Care Diagnostics) on a BCS® XP analyzer (Siemens Healthcare Diagnostics).

In some embodiments, the aPTT assay may also be used for assessing the potency of a chimeric polypeptide prior to administration to a subject. (Hubbard A R, et al. J Thromb Haemost 11: 988-9 (2013)). In some embodiments, the aPTT assay may further be used in conjunction with any of the assays described herein, either prior to administration or following administration to a subject.

In some embodiments, a model provided herein provides FVIII activity calculations and information that correspond to activity as measured by aPTT test. For example, efanesoctocog alfa popPK model [A] provides FVIII activity calculations and information that correspond to activity as measured by aPTT test.

### Subject PK and Dosing Information

An efanesoctocog alfa popPK model was developed using pooled FVIII activity data from efanesoctocog alfa Phase 1/2a and Phase 3 studies in adult, adolescent and pediatric patients with hemophilia A, in which PK information (FVIII activity) was adequately characterized by a one-compartment base structural model with linear elimination. The clearance (CL) and volume of central compartment (V) in the popPK model, depended on bodyweight, while Asian race was identified as significant covariate on CL. The efanesoctocog alfa popPK model [A] is represented as follows:

$$CL = TVCL * {( WT / 78.3 )}^{0.677} * {( 0.896 )}^{ASIAN} * {e}^{{η}^{1}} \qquad [ A ]$$

$$V = TVV * {( WT / 78.3 )}^{0.791} * {e}^{{η}^{2}}$$

$$k = CL / V$$

$$\frac{{dA}_{1}}{dt} = Rate - {kA}_{1}$$

$$C = {A}_{1} / V$$

Efanesoctocog alfa popPK model [A](also referred to herein as “popPK model [A]”) abbreviations are: CL, clearance from central compartment; TVCL, estimate of typical clearance; WT, bodyweight in kg; ASIAN, indicator for Asian race (0 for Non-Asians and 1 for Asians); η₁, variability on CL; V, volume of central compartment; TW, estimate of typical volume; η₂, variability on V; k, elimination rate from central compartment; Rate, rate of infusion; A₁, amount of one-stage (OS) FVIII activity in central compartment; and C, one-stage (OS) FVIII activity in central compartment.

A version of popPK model [A] containing parameters that are useful for patients of all ages can be described as popPK model [A′]:

$$CL = 0.433 {( \frac{Bodyweight ( kg )}{78.3} )}^{0.677} {( 0.896 )}^{Asian} {e}^{{η}_{1}} \qquad [ {A}^{′} ]$$

$$V = 30.2 {( \frac{Bodyweight ( kg )}{78.3} )}^{0.791} {e}^{{η}_{2}}$$

In some embodiments, efanesoctocog alfa model [A] is used to obtain PK information for a subject who is less than 6 years old. In some embodiments, efanesoctocog alfa model [A] is used to obtain PK information for a subject who is less than 12 years old. In some embodiments, efanesoctocog alfa model [A] is used to obtain PK information for a subject who is at least 12 years old. In some embodiments, efanesoctocog alfa model [A] is used to obtain PK information for a subject who is at least 18 years old.

In some embodiments, efanesoctocog alfa model [A′] is used to obtain PK information for a subject who is less than 6 years old. In some embodiments, efanesoctocog alfa model [A′] is used to obtain PK information for a subject who is less than 12 years old. In some embodiments, efanesoctocog alfa model [A′] is used to obtain PK information for a subject who is at least 12 years old. In some embodiments, efanesoctocog alfa model [A′] is used to obtain PK information for a subject who is at least 18 years old.

In some embodiments, the PK information is used in an RTTE model provided herein.

In some embodiments, efanesoctocog alfa model [A] is used to obtain dose information for a subject who is less than 6 years old. In some embodiments, efanesoctocog alfa model [A] is used to obtain dose information for a subject who is less than 12 years old. In some embodiments, efanesoctocog alfa model [A] is used to obtain dose information for a subject who is at least 12 years old. In some embodiments, efanesoctocog alfa model [A] is used to obtain dose information for a subject who is at least 18 years old.

In some embodiments, efanesoctocog alfa model [A′] is used to obtain dose information for a subject who is less than 6 years old. In some embodiments, efanesoctocog alfa model [A′] is used to obtain dose information for a subject who is less than 12 years old. In some embodiments, efanesoctocog alfa model [A′] is used to obtain dose information for a subject who is at least 12 years old. In some embodiments, efanesoctocog alfa model [A′] is used to obtain dose information for a subject who is at least 18 years old.

Some embodiments comprise administering a dose of efanesoctocog alfa to a human subject in need thereof at a dosing interval, wherein the dose and/or the dosing interval is identified using the subject's body weight and/or self-reported race, but not the subject's VWF or hematocrit levels. The present disclosure provides methods of administering a dose of efanesoctocog alfa to a human subject in need thereof at a dosing interval, wherein the dose and/or the dosing interval is identified by applying the efanesoctocog alfa model [A] disclosed herein. In some embodiments, the efanesoctocog model [A′] is used.

In some embodiments, the therapeutically effective dose of efanesoctocog alfa is about 50 IU/kg. In some embodiments, the subject is administered a dose of about 50 IU/kg once-weekly. In some embodiments, the subject is administered a dose of about 50 IU/kg once every about 7 days. In some embodiments, the subject is administered an initial dose of about 50 IU/kg, followed by either 50 IU/kg or 30 IU/kg every 2-3 days as needed.

In some embodiments, the methods disclosed herein are applied to determine a subject's individualized interval prophylaxis. The term “individualized interval prophylaxis” as used herein means use of efanesoctocog alfa for an individualized dose and/or dosing interval or frequency to prevent or inhibit occurrence of one or more spontaneous and/or uncontrollable bleeding or bleeding episodes or to reduce the frequency of one or more spontaneous and/or uncontrollable bleeding or bleeding episodes.

In some embodiments, subject treatment goals include achieving high FVIII plasma activity levels and/or high trough levels. As used herein, a “trough level” in a hemophilia subject is the measurement of the lowest concentration reached by a factor therapy, e.g., efanesoctocog alfa therapy, before the next dose is administered. The methods disclosed herein can be used to determine subject dosing information in order to achieve specific FVIII plasma activity levels and/or trough levels. Administration of efanesoctocog alfa has been shown to successfully achieve high FVIII plasma activity levels and/or high trough levels in hemophilia A subjects.

In some embodiments, administration of efanesoctocog alfa results in a level of FVIII activity of 40% or greater in the subject for about 1, 2, 3, or 4 days. In some embodiments, administration of efanesoctocog alfa results a level of FVIII activity of greater in the subject for about 1, 2, 3, or 4 days. In some embodiments, administration of efanesoctocog alfa results in a level of FVIII activity of at least 40% in the subject for at least three days. In some embodiments, administration of efanesoctocog alfa results in a level of FVIII activity of at least 50% in the subject for about 4 days. In some embodiments, the efanesoctocog alfa model [A] is used to determine individual subject dosing information in order to achieve a FVIII activity level of at least 40% in the subject for about 1, 2, 3 or 4 days.

### Estimating Bleed Risk

Included herein are Repeated Time to Event (RTTE) models for assessing the bleed risk of subjects with hemophilia A. In some embodiments, treatment effect (e.g., prophylaxis or on-demand treatment) is a covariate for base hazard in the RTTE model.

In the XTEND-1 Phase 3 study, bleeding events were recorded for subjects treated with efanesoctocog alfa either as on-demand or prophylactically (i.e. treatment effect). The information about the subject's bleeding profile and effect of FVIII activity (from efanesoctocog alfa dosing) was used to develop an exemplary RTTE model and identify demographic or disease history parameters that affect the bleeding profile.

Provided herein is a RTTE model [B] for estimating or predicting bleed risk. In some embodiments, the bleed risk is for a subject receiving FVIII replacement therapy. In some embodiments, the bleed risk is for a subject receiving FVIII replacement therapy with efanesoctocog alfa. The RTTE model [B] is represented as follows:

$$h ( t ) = λ {γ ( λ t )}^{γ - 1} {e}^{β {C}^{α}} {e}^{η} \qquad [ B ]$$

RTTE model [B] Abbreviations are: h: hazard function of Weibull distribution; t: time; λ: constant of the base hazard; γ: shape parameter of the base hazard; β: constant of the FVIII effect; C: FVIII activity; α: shape parameter of the FVIII effect; η: individual random effect. η varies with a mean of 0 and a coefficient of variation of 114%. Additional information relating to RTTE model [B] is provided in Example 2.

In some embodiments, η=0, resulting in RTTE model [C]. The RTTE model [C] is represented as follows:

$$h ( t ) = {λγ ( λ t )}^{γ - 1} {e}^{β {C}^{α}} \qquad [ C ]$$

RTTE model [C] Abbreviations are: h: hazard function of Weibull distribution; t: time; λ: constant of the base hazard; γ: shape parameter of the base hazard; β: constant of the FVIII effect; C: FVIII activity; α: shape parameter of the FVIII effect.

The instantaneous hazard from time 0 using the final RTTE model parameters is shown in models/equations [D] and [D1]:

$$h ( t ) = ( λγ ) {( λ t )}^{γ - 1} ( {e}^{On - demand - effect * ARM + β * {C}_{pred}^{α} ( t )} ) \qquad [ D ]$$

$$h ( t ) = ( 0.00247 * 1.02 ) * ( {( 0.00247 * t )}^{0.02} ) * {e}^{2.21 * ARM - 1.73 * {C}_{pred}^{0.247} ( t )} \qquad [ D1 ]$$

$$h ( t ) = ( 0.0000429 * 0.694 ) * ( {( 0.0000429 * t )}^{- 0.306} ) * {e}^{- 0.00535 * {C}_{pred}^{0.00429} ( t )} \qquad [ D2 ]$$

Abbreviations for equations [D], [D1], and [D2]: h, hazard function of Weibull distribution; t, time in hours; C_{pred} (t), predicted FVIII activity at time t; ARM is treatment effect, such that ARM is 1 if the subject is on on-demand treatment, or 0 if the subject is on prophylactic treatment. Equation [D1] contains the parameters for patients who are at least 12 years old. Equation [D2] contains the parameters for patients who are less than 12 years old.

In some embodiments, the RTTE model comprises treatment effect as a coviariate. In some embodiments, treatment effect is prophylaxis. In some embodiments, treatment effect is on-demand treatment. In some embodiments, the method comprises use of a RTTE model [B]. In some embodiments, η=0.

In some embodiments, the RTTE model [B] is used to estimate the bleed risk in an individual subject.

In some embodiments, the RTTE model [C] is used to estimate the bleed risk in an individual subject.

In some embodiments, the RTTE model [D] is used to estimate the bleed risk in an individual subject.

In some embodiments, the RTTE model [D1] is used to estimate the bleed risk in an individual subject. In some embodiments, RTTE model [D1] is used to estimate the bleed risk in an individual subject who is at least 12 years old. In some embodiments, RTTE model [D1] is used to estimate the bleed risk in an individual subject who is at least 18 years old.

In some embodiments, the RTTE model [D2] is used to estimate the bleed risk in an individual subject. In some embodiments, RTTE model [D2] is used to estimate the bleed risk in an individual subject who is less than 12 years old. In some embodiments, RTTE model [D2] is used to estimate the bleed risk in an individual subject who is at least 6 but less than 12 years old. In some embodiments, RTTE model [D2] is used to estimate the bleed risk in an individual subject who is no more than 6 years old.

In some embodiments, the RTTE model [B] is used to estimate the probability that a subject will have a bleed at a specific time point (t). In some embodiments, the RTTE model [C] is used to estimate the probability of a subject having a bleeding event within a period of time, such as within or over the course of 1 month, 3 months, 6 months, 1 year, 2, years, or more. In some embodiments, the RTTE model [B] is used to estimate the probability of a subject having a bleeding event within 1 year.

In some embodiments, the FVIII activity (C) in the RTTE model [B] is determined using the popPK model [A] disclosed herein. In some embodiments, the popPK model [A] and the RTTE model [B] can be used sequentially to estimate the bleed risk in a subject. In some embodiments, the risk of a bleeding event in a subject is predicted using the subject's body weight and/or self-reported race, but not the subject's VWF or hematocrit levels.

In some embodiments, the FVIII activity (C) in the RTTE model [B] is not determined using the popPK model [A] disclosed herein.

In some embodiments, the FVIII activity (C) in the RTTE model [B] is provided by the subject. In some embodiments, the FVIII activity (C) is a selected FVIII activity level. In some embodiments, the FVIII activity (C) is a target FVIII activity level. In some embodiments, the target FVIII activity level is a variable FVIII activity level. In some embodiments, target FVIII activity level is a constant FVIII activity level. In some embodiments, the RTTE model [B] is used to estimate the bleed risk of an individualized subject at a selected FVIII activity level. In some embodiments, the RTTE model [B] is used to estimate the bleed risk of an individualized subject at a target FVIII activity level.

In some embodiments, a desired treatment outcome is used in the RTTE model to estimate bleed risk. In some embodiments, the desired treatment outcome is provided by the individual subject.

In some embodiments, the RTTE model [B] is used to estimate the bleed risk in a subject with high sustained FVIII activity. In some embodiments, the RTTE model [B] is used to estimate the bleed risk in a subject with >40 IU/dL FVIII activity. In some embodiments, the RTTE model [B] is used to estimate the bleed risk in a subject with >40 IU/dL FVIII activity for about about 1, 2, 3, or 4 days. In some embodiments, the RTTE model [B] is used to estimate the bleed risk in a subject with >10 IU/dL FVIII activity. In some embodiments, the RTTE model [B] is used to estimate the bleed risk in a subject with >10 IU/dL FVIII activity for about about 1, 2, 3, 4, 5, 6, or 7 days. In some embodiments, the RTTE model [B] is used to estimate the bleed risk in a subject with a trough level of >10 IU/dL FVIII activity.

In some embodiments, the RTTE model [B] is used to determine the probability of a subject being bleed-free for 1 year with efanesoctocog alfa treatment. In some embodiments, the efanesoctocog alfa treatment comprises a dose of about 50 IU/kg of efanesoctocog alfa once weekly.

In some embodiments, the bleed risk information produced by the RTTE model [B] is used at least in part for selecting a treatments (e.g., a particular hemophilia A therapeutic and/or a dose regimen) for a hemophilia A subject. The risk information produced by the RTTE model [B] with efanesoctocog alfa treatment can be compared with the risk information using other hemophilia A treatments. Other hemophilia A treatments, include, for example, treatment with a continuous infusion regimen (CIR), treatment with efanesoctocog alfa, treatment with a recombinant clotting factor replacement product other than efanesoctocog alfa, treatment with a bispecific antibody that binds activated clotting Factor IX and clotting Factor X (such as emicizumab), or gene therapy for hemophilia A (such as valoctocogene roxaparvovec).

In some embodiments, disclosed herein is a method of treating a subject with hemophila A, the method comprising a) identifying a subject with hemophilia A; b) estimating the bleed risk in the subject when treated with 50 IU/kg efanesoctocog alfa about once weekly; c) estimating the bleed risk in the subject when treated with an alternative hemophilia A therapy that maintains a FVIII activity level of at least 10 IU/dL in the subject; and d) treating the subject with 50 IU/kg efanesoctocog alfa about once weekly if the subject has a lower estimated bleed risk with the efanesoctocog alfa therapy than the estimated bleed risk with the alternative hemophilia A therapy.

In some embodiments, the RTTE model [B] can be used without inputting any individualized FVIII activity data.

In some embodiments, the RTTE model [B] can be used to estimate the bleed risk in a population of subjects. In some embodiments, the estimated bleed risk is based on the population mean or median rather than being individualized for a particular subject.

In some embodiments, the RTTE model [B] can be used to estimate the bleed risk in a virtual or hypothetical subject.

In some embodiments, the bleed risk information produced by the RTTE model [B] is used to determine the added benefits of efanesoctocog alfa treatment in an individual subject.

In some embodiments, the RTTE model [B] is used to estimate the bleed risk in an individual subject.

### Method, System, and Storage Medium for Estimating Subject Individualized Bleed Risk Information

Included herein is a method of estimating (e.g., calculating, determining, or providing) bleed risk with efanesoctocog alfa treatment for an individual subject, the method comprising: (a) receiving subject information and/or desired treatment outcome information by a software-based system comprising a computer program programmed to operate with an efanesoctocog alfa RTTE model (e.g., efanesoctocog alfa RTTE model [B]), b) calculating individualized bleed risk information for the subject. In some embodiments, the program is also programmed to operate with an efanesoctocog alfa popPK model (e.g., efanesoctocog alfa popPK model [A]). One or more of the above steps may be performed using one or more of a software-based system, a network-based system, a computing system, or various combinations of the aforementioned-systems. For example, the exemplary network-based system can be used for obtaining an estimated subject individualized bleeding risk information.

In some embodiments, the methods disclosed herein further comprise receiving, by the software-based system, subject information. In some embodiments the subject information comprises age, self-reported race, and/or body weight. In some embodiments the subject information comprises diagnostic (baseline) FVIII level, PK determinations, time of PK sampling, dosing history if PK samples were taken from multiple doses, actual dose, FVIII activity level, etc.

In some embodiments, output information comprises estimated bleed risk. In some embodiments, output information comprises estimated bleed risk at a specific time (t). In some embodiments, output information comprises estimated risk of occurrence of a bleeding event. In some embodiments, output information comprises estimated risk of occurrence of a bleeding event within 1 year. In some embodiments, output information comprises estimated annual bleeding rate (ABR).

The system may be compliant with patient privacy laws. In some embodiments, the system is encrypted, e.g., with SSL. In some embodiments, input subject information is made anonymous.

In some embodiments, the system includes a user help function.

In some embodiments, the method can be carried out by, e.g., a subject, a physician, a nurse, or another healthcare practitioner. In some embodiments, the method is carried out by the subject.

Some embodiments include a computer readable storage medium having instructions stored thereon that, when executed by a processor, cause the processor to perform one or more steps of the above methods.

Some embodiments include a system comprising a processor and a memory, the memory having instructions stored thereon that, when executed by the processor, cause the processor to perform any of the above methods.

The user of the system or computer readable storage medium can be, e.g., a subject or a caregiver, or a physician, a nurse, or other healthcare practitioner.

In some embodiments, the subject information entered into the system includes body weight. In some embodiments, the subject information entered into the system is self-reported race. In some embodiments, the subject information entered into the system is FVIII activity levels. In some embodiments, the subject information entered into the system is target FVIII activity levels. In some embodiments, the subject information entered into the system is treatment effect (prophylaxis or on-demand treatment). In some embodiments, the methods disclosed herein include an electronic device. An electronic device can include, but is not limited to, a device having a processor and memory for executing and storing instructions. The electronic device may also include a display and one or more computer input devices such as a keyboard, a mouse, a pad, a touch screen, a microphone, and/or a joystick. In some embodiments, the electronic device is a general-purpose computing and data communication device such as digital pen, a smart phone, a smart watch, a tablet computer, a personal digital assistant, a handheld computer, a laptop computer, a point-of-sale transaction device, a scanner, a camera, and a fax machine. The electronic device may also have multiple processors and multiple shared or separate memory components. For example, the electronic device may be a clustered computing environment or server farm.

Alternatively, the electronic device can be a specialized data collection, computing and communications device such as, for example, a point-of-care (POC) device capable of receiving subject demographic information including age, vital signs including body weight, and/or blood characterizing values including self-reported race. The blood characterizing values may be received by the electronic device via a data communications channel, manual entry, and/or by diagnostic processes performed by the electronic device. Diagnostic processes performed on subject blood samples within the device may include ultrasound measurements, impedance measurements, conductivity measurements, and/or optical measurements. The electronic device may be further configured to receive, detect, record and/or communicate additional subject information including diagnostic (baseline) FVIII level, PK determinations, time of PK sampling, dosing history if PK samples were taken from multiple doses, actual dose, FVIII activity level, or treatment effect (prophylaxis or on-demand treatment). The electronic device communicates with one or more network-based (e.g., web-based) application programs over one or more networks, such as the Internet. Similar to the electronic device, the network-based (e.g., web-based) application program can be implemented using a general-purpose computer, a server, or other device capable of serving data to the electronic device. The electronic device can receive individualized subject efanesoctocog alfa PK information from a network-based (e.g., web-based) server and program. In some embodiments, the electronic device can assist in calculating the estimated bleed risk for an individualized subject, population, or other source.

The methods and systems described herein may be implemented in or via a mobile device. Mobile devices include navigation devices, mobile phones, smart phones, smart watches, tablets, mobile personal digital information processing terminals, laptops, palmtops, netbooks, pagers, electronic book terminals, music players, and the like. These devices, apart from other components, may comprise a storage medium such as flash memory, buffers, RAM, ROM and one or more computing devices. A computing device associated with the mobile device may be adapted to execute program code, methods, and instructions stored thereon. As another example, a mobile device may be configured to execute instructions in cooperation with other devices. The mobile device may communicate with a base station that is connected to the server and configured to execute the program code. Mobile devices can also communicate over peer-to-peer networks, mesh networks, or other communication networks. The program code may be stored in a storage medium associated with the server and executed by a computing device embedded in the server. The base station may comprise a computing device and a storage medium. The storage medium may store program code and instructions that are executed by a computing device associated with the base station. In some embodiments, the methods and systems described herein are directed to a kit for collecting subject information. Although different embodiments of the kit may include different components, an exemplary kit includes a diagnostic device such as a processing element and/or a calculation element for acquiring information from the subject, and a transmission element that transmits the subject information to a computer device through a wired or wireless connection. The transmitting element in the kit may be configured to transmit subject information in real time when the device is in use, or the diagnostic information may be transmitted with receipt of instructions from a user or provider. Any of the components of the kit, such as the body, can be configured as a hands-free unit during use or as a handheld unit during use.

### Exemplary Computing Environments for the Disclosed Methods and Systems

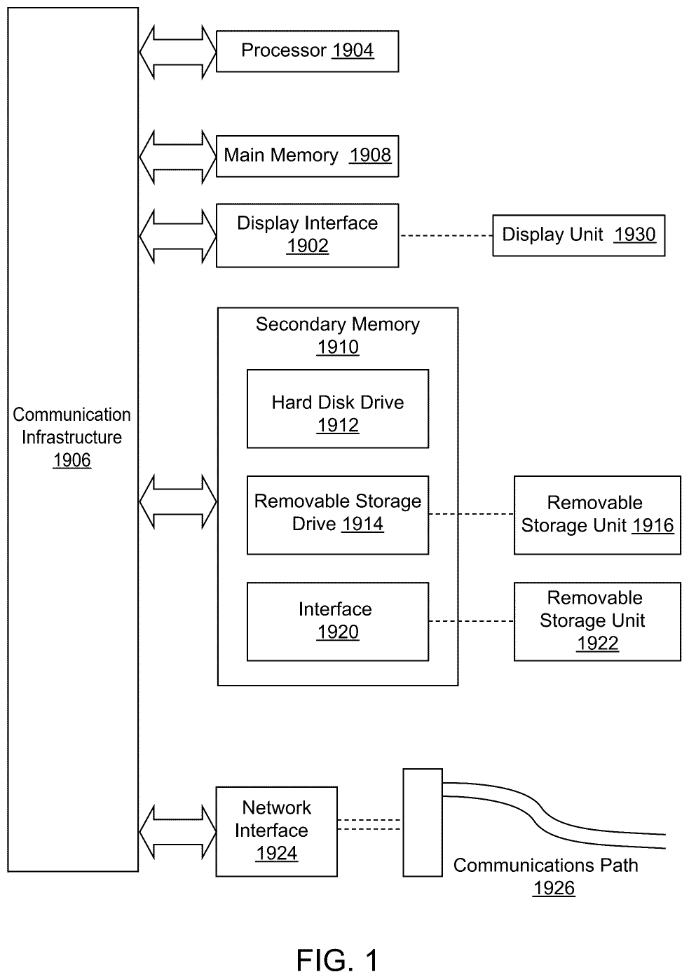

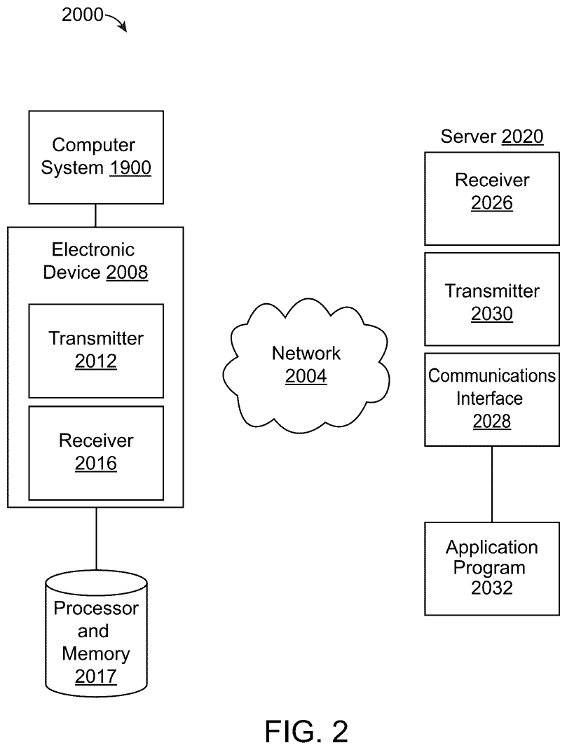

Various modeling techniques, dosage calculations, and estimations described herein can be implemented by software, firmware, hardware, or a combination thereof. FIG. 1 illustrates an example software-based computer system 1900 in which the embodiments, or portions thereof, can be implemented as computer-readable code. In another embodiment, for efanesoctocog alfa, the modeling disclosed in the Examples herein can be implemented in system 1900. Computer system 1900 includes one or more processors, such as processor 1904. Processor 1904 is connected to a communication infrastructure 1906 (for example, a bus or network).

Computer system 1900 also includes a main memory 1908, preferably random access memory (RAM), and may also include a secondary memory 1910. In accordance with implementations, user interface data may be stored, for example and without limitation, in main memory 1908. Main memory 1908 may include, for example, cache, and/or static and/or dynamic RAM. Secondary memory 1910 may include, for example, a hard disk drive and/or a removable storage drive. Removable storage drive 1914 may include a floppy disk drive, a magnetic tape drive, an optical disk drive, a flash memory, or the like. The removable storage drive 1914 reads from and/or writes to removable storage unit 1916 in a well-known manner. Removable storage unit 1916 may include a floppy disk, magnetic tape, optical disk, etc. which is read by and written to by removable storage drive 1914. As will be appreciated by persons skilled in the relevant art(s), removable storage unit 1916 includes a computer readable storage medium having stored therein computer software and/or data.

Computer system 1900 may also include a display interface 1902. Display interface 1902 may be adapted to communicate with display unit 1930. Display unit 1930 may include a computer monitor or similar means for displaying graphics, text, and other data received from main memory 1908 via communication infrastructure 1906. In alternative implementations, secondary memory 1910 may include other similar means for allowing computer programs or other instructions to be loaded into computer system 1900. Such means may include, for example, a removable storage unit 1922 and an interface 1920. Examples of such means may include a program cartridge and cartridge interface, a removable memory chip (such as an EPROM, or PROM) and associated socket, and other removable storage units 1922 and interfaces 1920 which allow software and data to be transferred from the removable storage unit 1922 to computer system 1900.

Computer system 1900 may also include a communications interface 1924. Communications interface 1924 allows software and data to be transferred between computer system 1900 and external devices. Communications interface 1924 may include a modem, a network interface (such as an Ethernet card or WiFi), a communications port, a PCMCIA slot and card, or the like. Software and data transferred via communications interface 1924 are in the form of signals which may be electronic, electromagnetic, optical, or other signals capable of being received by communications interface 1924. These signals are provided to communications interface 1924 via a communications path 1926. Communications path 1926 carries signals and may be implemented using wire or cable, fiber optics, a phone line, a cellular phone link, WiFi, Bluetooth, an RF link or other communications channels.

In this document, the term “computer readable storage medium” is used to generally refer to non-transitory storage media such as removable storage unit 1916, removable storage unit 1922, and a hard disk installed in hard disk drive 1912. Computer readable storage medium can also refer to one or more memories, such as main memory 1908 and secondary memory 1910, which can be memory semiconductors (e.g., DRAMs, etc.). These computer program products are means for providing software to computer system 1900.

Computer programs (also called computer control logic) are stored in main memory 1908 and/or secondary memory 1910. Computer programs may also be received via communications interface 1924 and stored on main memory 1908 and/or secondary memory 1910. Such computer programs, when executed, enable computer system 1900 to implement embodiments as discussed herein. In particular, the computer programs, when executed, enable processor 1904 to implement processes of the present disclosure, such as certain methods discussed above. Accordingly, such computer programs represent controllers of the computer system 1900. Where embodiments use software, the software may be stored in a computer program product and loaded into computer system 1900 using removable storage drive 1914, interface 1920, or hard drive 1912.

Embodiments may be directed to computer program products comprising software stored on any computer readable medium, or distributed across several such media. Such software, when executed in one or more processing devices, causes a processing device to operate as described herein. Embodiments may employ any computer useable or readable medium. Examples of computer readable storage media include, but are not limited to, non-transitory primary storage devices (e.g., any type of random access memory), and non-transitory secondary storage devices (e.g., hard drives, floppy disks, CD ROMS, ZIP disks, tapes, magnetic storage devices, and optical storage devices, MEMS, nano-technological storage device, etc.). Other computer readable media include communication mediums (e.g., wired and wireless communications networks, local area networks, wide area networks, intranets, etc.).

Non-limiting examples of software-based systems include network-based systems and web-based systems.

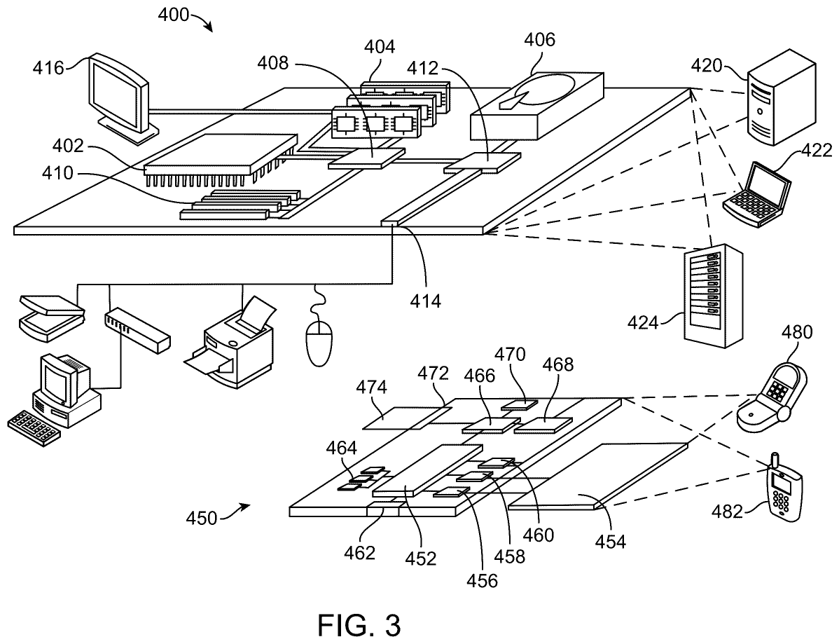

FIG. 3 shows an example of a computing device 400 and an example of a mobile computing device 450 that can be used to implement the techniques described here. A software-based system such as that described in FIG. 1 may be implemented in a computing device 400 or mobile computing device 450. The computing device 400 is intended to represent various forms of digital computers, such as laptops, desktops, workstations, personal digital assistants, servers, blade servers, mainframes, and other appropriate computers. The mobile computing device 450 is intended to represent various forms of mobile devices, such as personal digital assistants, tablets, cellular telephones, smart-phones, and other similar computing devices. The components shown here, their connections and relationships, and their functions, are meant to be exemplary only, and are not meant to limit implementations of the inventions described and/or claimed in this document.

The computing device 400 includes a processor 402, a memory 404, a storage device 406, a high-speed interface 408 connecting to the memory 404 and multiple high-speed expansion ports 410, and a low-speed interface 412 connecting to a low-speed expansion port 414 and the storage device 406. Each of the processor 402, the memory 404, the storage device 406, the high-speed interface 408, the high-speed expansion ports 410, and the low-speed interface 412, are interconnected using various busses, and can be mounted on a common motherboard or in other manners as appropriate. The processor 402 can process instructions for execution within the computing device 400, including instructions stored in the memory 404 or on the storage device 406 to transmit and display graphical or other information for a GUI on an external input/output device, such as a display 416 coupled to the high-speed interface 408. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 404 stores information within the computing device 400. In some implementations, the memory 404 is a volatile memory unit or units. In some implementations, the memory 404 is a non-volatile memory unit or units. The memory 404 can also be another form of computer-readable medium, such as a magnetic or optical disk.

The storage device 406 is capable of providing mass storage for the computing device 400. In some implementations, the storage device 406 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 404, the storage device 406, or memory on the processor 402.

The high-speed interface 408 manages bandwidth-intensive operations for the computing device 400, while the low-speed interface 412 manages lower bandwidth-intensive operations. Such allocation of functions is exemplary only. In some implementations, the high-speed interface 408 is coupled to the memory 404, the display 416 (e.g., through a graphics processor or accelerator), and to the high-speed expansion ports 410, which can accept various expansion cards (not shown). In the implementation, the low-speed interface 412 is coupled to the storage device 406 and the low-speed expansion port 414. The low-speed expansion port 414, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 400 can be implemented in a number of different forms, as shown in the figure. For example, it can be implemented as a standard server 420, or multiple times in a group of such servers. In addition, it can be implemented in a personal computer such as a laptop computer 422. It can also be implemented as part of a rack server system 424. Alternatively, components from the computing device 400 can be combined with other components in a mobile device (not shown), such as a mobile computing device 450. Each of such devices can contain one or more of the computing device 400 and the mobile computing device 450, and an entire system can be made up of multiple computing devices communicating with each other.

The mobile computing device 450 includes a processor 452, a memory 464, an input/output device such as a display 454, a communication interface 466, and a transceiver 468, among other components. The mobile computing device 450 can also be provided with a storage device, such as a micro-drive or other device, to provide additional storage. Each of the processor 452, the memory 464, the display 454, the communication interface 466, and the transceiver 468, are interconnected using various buses, and several of the components can be mounted on a common motherboard or in other manners as appropriate.

The processor 452 can execute instructions within the mobile computing device 450, including instructions stored in the memory 464. The processor 452 can be implemented as a chipset of chips that include separate and multiple analog and digital processors. The processor 452 can provide, for example, for coordination of the other components of the mobile computing device 450, such as control of user interfaces, applications run by the mobile computing device 450, and wireless communication by the mobile computing device 450.

The processor 452 can communicate with a user through a control interface 458 and a display interface 456 coupled to the display 454. The display 454 can be, for example, a TFT (Thin-Film-Transistor Liquid Crystal Display) display or an OLED (Organic Light Emitting Diode) display, or other appropriate display technology. The display interface 456 can comprise appropriate circuitry for driving the display 454 to present graphical and other information to a user. The control interface 458 can receive commands from a user and convert them for submission to the processor 452. In addition, an external interface 462 can provide communication with the processor 452, so as to enable near area communication of the mobile computing device 450 with other devices. The external interface 462 can provide, for example, for wired communication in some implementations, or for wireless communication in other implementations, and multiple interfaces can also be used.

The memory 464 stores information within the mobile computing device 450. The memory 464 can be implemented as one or more of a computer-readable medium or media, a volatile memory unit or units, or a non-volatile memory unit or units. An expansion memory 474 can also be provided and connected to the mobile computing device 450 through an expansion interface 472, which can include, for example, a SIMM (Single In Line Memory Module) card interface. The expansion memory 474 can provide extra storage space for the mobile computing device 450, or can also store applications or other information for the mobile computing device 450. Specifically, the expansion memory 474 can include instructions to carry out or supplement the processes described above, and can include secure information also. Thus, for example, the expansion memory 474 can be provide as a security module for the mobile computing device 450, and can be programmed with instructions that permit secure use of the mobile computing device 450. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 464, the expansion memory 474, or memory on the processor 452. In some implementations, the computer program product can be received in a propagated signal, for example, over the transceiver 468 or the external interface 462.

The mobile computing device 450 can communicate wirelessly through the communication interface 466, which can include digital signal processing circuitry where necessary. The communication interface 466 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 468 using a radio-frequency. In addition, short-range communication can occur, such as using a Bluetooth, WiFi, or other such transceiver (not shown). In addition, a GPS (Global Positioning System) receiver module 470 can provide additional navigation- and location-related wireless data to the mobile computing device 450, which can be used as appropriate by applications running on the mobile computing device 450.

The mobile computing device 450 can also communicate audibly using an audio codec 460, which can receive spoken information from a user and convert it to usable digital information. The audio codec 460 can likewise generate audible sound for a user, such as through a speaker, e.g., in a handset of the mobile computing device 450. Such sound can include sound from voice telephone calls, can include recorded sound (e.g., voice messages, music files, etc.) and can also include sound generated by applications operating on the mobile computing device 450.

The mobile computing device 450 can be implemented in a number of different forms, as shown in the figure. For example, it can be implemented as a cellular telephone 480. It can also be implemented as part of a smart-phone 482, personal digital assistant, a tablet, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard, pointing device (e.g., a mouse or a trackball), and/or touchscreen or other user interface, by which the user can provide input to the computer and to a software-based system implemented on the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

### EMBODIMENTS OF THE DISCLOSURE

The present disclosure includes (and is not limited to) the following exemplary embodiments:
E1. A method of estimating bleed risk, the method comprising: (a) receiving clotting Factor VIII (FVIII) activity information by a software-based system comprising a computer program that is programmed to implement a Repeated Time to Event (RTTE) model [B], (b) calculating, by the computer program, bleed risk using the RTTE model [B] and the received information, and (c) transmitting, by the software-based system, the calculated bleed risk information of (b) for output of the bleed risk information.
E2. A method of estimating bleed risk, the method comprising: (a) receiving, by one or more electronic devices, clotting Factor VIII (FVIII) activity information, (b) transmitting, by a processing device, the clotting Factor VIII (FVIII) information to a software-based system, wherein the software-based system is programmed to implement a Repeated Time to Event (RTTE) model [B] to calculate bleed risk; (c) receiving, from the software-based system, bleed risk information calculated using the transmitted information of (b) and the RTTE model [B]; and (d) transmitting, by the one or more electronic devices, the bleed risk information of (c) for output of the bleed risk information.
E3. The method of embodiment 1 or 2, wherein calculating bleed risk is estimating the risk of occurrence of a bleeding event.
E4. The method of any one of embodiments 1-3, wherein the FVIII activity information is provided by a popPK model.
E5. The method of embodiment 4, wherein the popPK model is an efanesoctocog alfa popPK model.
E6. The method of embodiment 5, wherein the efanesoctocog alfa popPK model comprises body weight as a covariate, and wherein the efanesoctocog alfa popPK model does not comprise the level of VWF or level of hematocrit as covariates.
E7. The method of embodiment 5, wherein the popPK model is an efanesoctocog alfa popPK model [A].
E8. The method of embodiment 5, wherein the popPK model is an efanesoctocog alfa popPK model [A′].
E9. The method of any one of embodiments 1-8, wherein the FVIII activity information provided is for an individual subject.
E10. The method of any one of embodiments 1-3, wherein the FVIII activity information provided is a target FVIII activity level.
E11. The method of embodiment 10, wherein the target FVIII activity level is a variable FVIII activity level.
E12. The method of any one of embodiments 1-8, wherein the FVIII activity information provided is the estimated FVIII activity level of a population of subjects.
E13. The method of any one of embodiments 1-8, wherein the FVIII activity information provided is for a virtual or hypothetical subject.
E14. The method of any one of embodiments 1-13, wherein the RTTE model is the RTTE model [C].
E15. The method of any one of embodiments 1-13, wherein the RTTE model is the RTTE model [D].
E16. The method of any one of embodiments 1-13, wherein the RTTE model is the RTTE model [D1].
E17. The method of any one of embodiments 1-13, wherein the RTTE model is the RTTE model [D2].
E18. A method for estimating bleed risk for an individual subject, the method comprising a) receiving, by a software-based system, individualized subject information comprising the subject's body weight; b) receiving, by the software-based system, desired treatment outcome information comprising a FVIII level; c) applying a repeated time to event (RTTE) model for the subject based on the individualized information and/or the desired treatment outcome information; d) estimating bleed risk for the individual subject using the RTTE model; and e) transmitting, by the software-based system, the estimated bleed risk information of (d) for output of the bleed risk information.
E19. The method of embodiment 18, wherein the RTTE model comprises treatment effect as a covariate.
E20. The method of embodiment 19, wherein the treatment effect is prophylaxis treatment or on-demand treatment.
E21. The method of any one of embodiments 18-20, wherein the RTTE model is the RTTE model [B].
E22. The method of any one of embodiments 18-20, wherein the RTTE model is the RTTE model [C].
E23. The method of any one of embodiments 18-20, wherein the RTTE model is the RTTE model [D].
E24. The method of any one of embodiments 18-20, wherein the RTTE model is the RTTE model [D1].
E25. The method of any one of embodiments 18-20, wherein the RTTE model is the RTTE model [D2].
E26. The method of any one of embodiments 18-25, wherein the desired treatment outcome information is provided by the efanesoctocog alfa popPK model [A].
E27. The method of any one of embodiments 18-25, wherein the desired treatment outcome information is provided by the efanesoctocog alfa popPK model [A′].
E28. The method of any one of embodiments 18-25, wherein the desired treatment outcome information is provided by the individual subject.
E29. The method of any one of embodiments 18-25, wherein the desired treatment outcome information is provided by a healthcare professional.
E30. A method of treating a subject with hemophila A, the method comprising: a) identifying a subject with hemophilia A; b) estimating the bleed risk in the subject when treated with 50 IU/kg efanesoctocog alfa about once weekly, c) estimating the bleed risk in the subject if the subject had FVIII activity level of at least 10 IU/dL; and d) treating the subject with 50 IU/kg efanesoctocog alfa about once weekly if the subject has a lower estimated bleed risk with the efanesoctocog alfa therapy than the estimated bleed risk if the subject had a FVIII activity level of at least 10 IU/dL. E31. The method of embodiment 30, wherein a Repeated Time to Event (RTTE) model is used to estimate the bleed risk of the efanesoctocog alfa therapy and/or the bleed risk in the subject if the subject had FVIII activity level of at least 10 IU/dL.
E32. The method of embodiment 31, wherein the RTTE model is RTTE model [B].
E33. The method of embodiment 31, wherein the RTTE model is RTTE model [C].
E34. The method of embodiment 31, wherein the RTTE model is RTTE model [D].
E35. The method of embodiment 31, wherein the RTTE model is RTTE model [D1].
E36. The method of embodiment 31, wherein the RTTE model is RTTE model [D2].
E37. A method for estimating bleed risk for an individual subject, the method comprising: (a) receiving information for an individual subject by a software-based system, wherein the system is programmed to implement: (i) a one-compartment efanesoctocog alfa popPK model to calculate clotting Factor VIII (FVIII) activity information, wherein the efanesoctocog alfa popPK model comprises body weight as a covariate, and wherein the efanesoctocog alfa popPK model does not comprise the level of VWF or level of hematocrit as covariates; and (ii) a Repeated Time to Event (RTTE) model that uses the FVIII activity information to estimate bleed risk, (b) calculating, by the software-based system, estimated bleed risk using the efanesoctocog alfa popPK model, the RTTE model, and the received information, and (c) transmitting, by the software-based system, the calculated bleed risk information of (b) for output of the bleed risk information.
E38. A method of estimating bleed risk, the method comprising: (a) receiving, by one or more electronic devices, information for an individual subject; (b) transmitting, by a processing device, information for an individual subject to a software-based system, wherein the software-based system is programmed to implement (i) a one-compartment efanesoctocog alfa popPK model to calculate clotting Factor VIII (FVIII) activity information, wherein the efanesoctocog alfa popPK model comprises body weight as a covariate, and wherein the efanesoctocog alfa popPK model does not comprise the level of VWF or level of hematocrit as covariates; and (ii) a Repeated Time to Event (RTTE) model that uses the FVIII activity information to estimate bleed risk, (c) receiving, from the software-based system, estimated bleed risk using the efanesoctocog alfa popPK model, the RTTE model, and the received information; and (d) transmitting, by the one or more electronic devices, the bleed risk information of (c) for output of the bleed risk information.
E39. The method of embodiment 37 or 38, wherein the RTTE model is RTTE model [B].
E40. The method of embodiment 37 or 38, wherein the RTTE model is RTTE model [C].
E41. The method of embodiment 37 or 38, wherein the RTTE model is RTTE model [D].
E42. The method of embodiment 37 or 38, wherein the RTTE model is RTTE model [D1].
E43. The method of embodiment 37 or 38, wherein the RTTE model is RTTE model [D2].
E44. The method of any one of embodiments 38-43, wherein the efanesoctocog alfa popPK model is the efanesoctocog alfa popPK model [A].
E45. The method of any one of embodiments 38-43, wherein the efanesoctocog alfa popPK model is the efanesoctocog alfa popPK model [A′].
E46. The method of any one of embodiments 1-45, wherein the subject is at least 12 years old.
E47. The method of any one of embodiments 1-45, wherein the subject is at least 18 years old.
E48. The method of any one of embodiments 1-45, wherein the wherein the subject is less than 12 years old.
E49. The method of any one of embodiments 1-45, wherein the wherein the subject is less than 6 years old.
E50. The method of any one of embodiments 1-45, wherein the wherein the subject is between 6 and 12 years old.
E51. A data processing apparatus, device, or system comprising a processor configured to implement an efanesoctocog alfa RTTE model.
E52. The data processing apparatus, device, or system of embodiment 51, wherein the RTTE model is RTTE model [B].
E53. The data processing apparatus, device, or system of embodiment 51, wherein the RTTE model is RTTE model [C].
E54. The data processing apparatus, device, or system of embodiment 51, wherein the RTTE model is RTTE model [D].
E55. The data processing apparatus, device, or system of embodiment 51, wherein the RTTE model is RTTE model [D1].
E56. The data processing apparatus, device, or system of embodiment 51, wherein the RTTE model is RTTE model [D2].
E57. The data processing apparatus, device, or system of any one of embodiments 51-56 which is also configured to implement a one-compartment efanesoctocog alfa popPK model that comprises body weight as a covariate, and wherein the efanesoctocog alfa popPK model does not comprise the level of VWF or level of hematocrit as covariates.
E58. The data processing apparatus, device, or system of any one of embodiments 51-57, wherein the efanesoctocog alfa popPK model is efanesoctocog alfa popPK model [A].
E59. The data processing apparatus, device, or system of any one of embodiments 51-57, wherein the efanesoctocog alfa popPK model is efanesoctocog alfa popPK model [A′].
E60. The data processing apparatus, device, or system of any one of embodiments 51-59, which comprises a smart phone, a tablet computer, a personal digital assistant, a handheld computer, a laptop computer, or a smart watch.
E61. The data processing apparatus, device, or system of any one of embodiments 51-59, which comprises a smart phone.
E62. A computer program comprising instructions which, when the program is executed by a computer, cause the computer to implement an efanesoctocog alfa repeated time to event (RTTE) model.
E63. The computer program of embodiment 62, wherein the RTTE model is RTTE model [B].
E64. The computer program of embodiment 62, wherein the RTTE model is RTTE model [C].
E65. The computer program of embodiment 62, wherein the RTTE model is RTTE model [D].
E66. The computer program of embodiment 62, wherein the RTTE model is RTTE model [D1].
E67. The computer program of embodiment 62, wherein the RTTE model is RTTE model [D2].
E68. The computer program of any one of embodiments 62-67, wherein the RTTE model uses a PK profile generated from a popPK model as an input.
E69. The computer program of embodiment 68, wherein the popPK model is an efanesoctocog alfa popPK model.
E70. The computer program of embodiment 69, wherein the efanesoctocog alfa popPK model is a one-compartment efanesoctocog alfa popPK model that comprises body weight as a covariate, and wherein the efanesoctocog alfa popPK model does not comprise the level of VWF or level of hematocrit as covariates.
E71. The computer program of any one of embodiments 68-70, wherein the popPK model is efanesoctocog alfa popPK model [A].
E72. The computer program of any one of embodiments 68-70, wherein the popPK model is efanesoctocog alfa popPK model [A′].
E73. A computer-readable medium comprising instructions which, when executed by a computer, cause the computer to carry out the method of any one of embodiments 1-50.
E74. A method of estimating bleed risk, the method comprising: (a) receiving clotting Factor VIII (FVIII) activity information by a software-based system comprising a computer program that is programmed to implement a Repeated Time to Event (RTTE) model [D2], (b) calculating, by the computer program, bleed risk using the RTTE model [D2] and the received information, and (c) transmitting, by the software-based system, the calculated bleed risk information of (b) for output of the bleed risk information.
E75. A method of estimating bleed risk, the method comprising: (a) receiving, by one or more electronic devices, clotting Factor VIII (FVIII) activity information, (b) transmitting, by a processing device, the clotting Factor VIII (FVIII) information to a software-based system, wherein the software-based system is programmed to implement a Repeated Time to Event (RTTE) model [D2] to calculate bleed risk; (c) receiving, from the software-based system, bleed risk information calculated using the transmitted information of (b) and the RTTE model [D2]; and (d) transmitting, by the one or more electronic devices, the bleed risk information of (c) for output of the bleed risk information. E76. The method of embodiment 74 or 75, wherein calculating bleed risk is estimating the risk of occurrence of a bleeding event.
E77. The method of any one of embodiments 74-76, wherein the FVIII activity information is provided by a popPK model.
E78. The method of embodiment 77, wherein the popPK model is an efanesoctocog alfa popPK model.
E79. The method of embodiment 78, wherein the popPK model is an efanesoctocog alfa popPK model [A].
E80. The method of embodiment 78, wherein the popPK model is an efanesoctocog alfa popPK model [A′].
E81. The method of any one of embodiments 74-80, wherein the FVIII activity information provided is for an individual subject.
E82. The method of any one of embodiments 74-80, wherein the FVIII activity information provided is a target FVIII activity level.
E83. The method of embodiment 82, wherein the target FVIII activity level is a variable FVIII activity level.
E84. The method of any one of embodiments 74-80, wherein the FVIII activity information provided is the estimated FVIII activity level of a population of subjects.
E85. The method of any one of embodiments 74-80, wherein the FVIII activity information provided is for a virtual or hypothetical subject.
E86. A method for estimating bleed risk for an individual subject, the method comprising a) receiving, by a software-based system, individualized subject information comprising the subject's body weight; b) receiving, by the software-based system, desired treatment outcome information comprising a FVIII level; c) applying a Repeated Time to Event (RTTE) model [D2] for the subject based on the individualized information and/or the desired treatment outcome information; d) estimating bleed risk for the individual subject using the RTTE model [D2]; and e) transmitting, by the software-based system, the estimated bleed risk information of (d) for output of the bleed risk information.
E87. The method of embodiment 86, wherein the desired treatment outcome information is provided by the efanesoctocog alfa popPK model [A].
E88. The method of embodiment 86, wherein the desired treatment outcome information is provided by the efanesoctocog alfa popPK model [A′].
E89. The method of any one of embodiments 86-88, wherein the desired treatment outcome information is provided by the individual subject.
E90. The method of any one of embodiments 86-88, wherein the desired treatment outcome information is provided by a healthcare professional.
E91. A method of treating a subject with hemophila A, the method comprising: a) identifying a subject with hemophilia A; b) estimating the bleed risk in the subject when treated with 50 IU/kg efanesoctocog alfa about once weekly, c) estimating the bleed risk in the subject if the subject had FVIII activity level of at least 10 IU/dL; and d) treating the subject with 50 IU/kg efanesoctocog alfa about once weekly if the subject has a lower estimated bleed risk with the efanesoctocog alfa therapy than the estimated bleed risk if the subject had a FVIII activity level of at least 10 IU/dL, wherein Repeated Time to Event (RTTE) model [D2] is used to estimate the bleed risk of the efanesoctocog alfa therapy and/or the bleed risk in the subject if the subject had FVIII activity level of at least 10 IU/dL.
E92. A method for estimating bleed risk for an individual subject, the method comprising: (a) receiving information for an individual subject by a software-based system, wherein the system is programmed to implement: (i) a one-compartment efanesoctocog alfa popPK model [A′] to calculate clotting Factor VIII (FVIII) activity information, and (ii) a Repeated Time to Event (RTTE) model [D2] that uses the FVIII activity information to estimate bleed risk, (b) calculating, by the software-based system, estimated bleed risk using the efanesoctocog alfa popPK model [Q], the RTTE model [D2], and the received information, and (c) transmitting, by the software-based system, the calculated bleed risk information of (b) for output of the bleed risk information.
E93. A method of estimating bleed risk, the method comprising: (a) receiving, by one or more electronic devices, information for an individual subject; (b) transmitting, by a processing device, information for an individual subject to a software-based system, wherein the software-based system is programmed to implement (i) a one-compartment efanesoctocog alfa popPK model [A′] to calculate clotting Factor VIII (FVIII) activity information, and (ii) a Repeated Time to Event (RTTE) model [D2], that uses the FVIII activity information to estimate bleed risk, (c) receiving, from the software-based system, estimated bleed risk using the efanesoctocog alfa popPK popPK model [Q], the RTTE model [D2], and the received information; and (d) transmitting, by the one or more electronic devices, the bleed risk information of (c) for output of the bleed risk information.
E94. The method of any one of embodiments 91-93, wherein the subject is less than 12 years old.
E95. The method of any one of embodiments 91-93, wherein the subject is less than 6 years old.
E96. The method of any one of embodiments 91-93, wherein the subject is between 6 and 12 years old.
E97. A data processing apparatus, device, or system comprising a processor configured to implement efanesoctocog alfa RTTE model [D2].
E98. The data processing apparatus, device, or system of embodiment 97, which comprises a smart phone, a tablet computer, a personal digital assistant, a handheld computer, a laptop computer, or a smart watch.
E99. The data processing apparatus, device, or system of any one of embodiments 97-98, which comprises a smart phone.
E100. A computer program comprising instructions which, when the program is executed by a computer, cause the computer to implement efanesoctocog alfa repeated time to event (RTTE) model [D2].
E101. A computer-readable medium comprising instructions which, when executed by a computer, cause the computer to carry out the method of any one of embodiments 74-96.
E102. A pharmaceutical composition comprising efanesoctocog alfa for use in reducing the risk that a human subject with severe hemophilia A in need thereof will have a bleed that is not caused by trauma over a period of 52 weeks, wherein the risk is reduced to a probability of less than 30%, wherein from about 25 IU/kg to about 50 IU/kg of efanesoctocog alfa is intravenously administered to the subject every about 4 to about 14 days during the period, wherein the human subject is 6 to <12 years of age, and wherein the subject weighs from 30 to 35 kg intravenously administering to the subject, thereby reducing the probability that to less than 30%. E103. A pharmaceutical composition comprising efanesoctocog alfa for use in reducing the risk that a human subject with severe hemophilia A in need thereof will have a bleed that is not caused by trauma over a period of 52 weeks, wherein the risk is reduced to a probability of less than 30%, wherein from about 25 IU/kg to about 50 IU/kg of efanesoctocog alfa is intravenously administered to the subject every about 4 to about 14 days during the period, wherein the human subject is 6 to <12 years of age, and wherein the subject weighs from 30 to 35 kg, thereby reducing the probability to less than 30%.
E104. The pharmaceutical composition of any one of embodiments 102-103 wherein the bleed that is not caused by trauma is a spontaneous bleed.
E105. The pharmaceutical composition of any one of embodiments 102-104, wherein the efanesoctocog alfa is administered at a dose of about 25 IU/kg every about 4 days during the period.
E106. The pharmaceutical composition of any one of embodiments 102-104, wherein the efanesoctocog alfa is administered at a dose of about 30 IU/kg every about 7 days during the period.
E107. The pharmaceutical composition of any one of embodiments 102-104, wherein the efanesoctocog alfa is administered at a dose of about 50 IU/kg every about 7 days during the period.
E108. The pharmaceutical composition of any one of embodiments 102-104, wherein the efanesoctocog alfa is administered at a dose of about 50 IU/kg every about 14 days during the period.

Having now described the present disclosure in detail, the same will be more clearly understood by reference to the following examples, which are included herewith for purposes of illustration only and are not intended to be limiting of the present disclosure. All patents and publications referred to herein are expressly incorporated by reference.

### EXAMPLES

### Example 1. Repeated Time to Event (RTTE) Model to Characterize Bleed Risk in Patients with Severe Hemophilia a Treated with Efanesoctocog Alfa

The XTEND-1 (EFC16293, NCT04161495) study was a Phase 3, open-label, multicenter study of efanesoctocog alfa in previously treated adult and adolescent (>12 years) patients with severe hemophilia A. Some patients had been on prestudy FVIII prophylaxis, and these patients were were enrolled into Arm A, where they received 52 weeks of once-weekly 50 IU/kg prophylaxis. Patients who had received on-demand therapy before the XTEND-1 study entered Arm B and received 26 weeks of on-demand 50 IU/kg, followed by 26 weeks of once-weekly prophylaxis.

During the XTEND-1 study, treated bleeding events were recorded for patients receiving efanesoctocog alfa during the prophylaxis and on-demand treatment periods. These data were used to develop a repeated time to event (RTTE) model for efanesoctocog alfa as a means to quantitatively characterize the dependence of the occurrence of bleeding events on base hazard and FVIII activity during efanesoctocog alfa treatment in patients with severe hemophilia A. Though the RTTE model was developed for efanesoctocog alfa, it is also useful for existing FVIII replacement products (such as recombinant FVIII, fusion proteins comprising recombinant FVIII and Fc, and PEGylated FVIII) as well as for assessing bleed risk for hypothetical or desired FVIII activity levels.

The RTTE model was developed using NONMEM (v.7.4.4 or higher). A schematic of the RTTE model development is shown in Table 1.

TABLE 1
Schematic of RTTE model development

| | |
|---|---|
| Research an | The baseline hazard (disease progression) was |
| efanesoctocog alfa | first developed without including predicted |
| base hazard model | FVIII activity or covariates |
| | Exponential, Weibull, and Gompertz distributions |
| | were tested |
| Test FVIII effect | The influence of FVIII activity was assessed |
| in base model | by adding a FVIII activity effect model for the |
| | exposure-response relationship |
| | A power model, an Emax (maximum rate) model, |
| | and a sigmoidal Emax model were tested |
| Conduct covariate | Arm (on-demand or prophylaxis), age at the |
| research | start of first prophylaxis, number of target |
| | joints, and baseline hemophilia joint health |
| | score (HJHS) were the factors assessed in |
| | covariate research |

During the development of the model, different models of hazard distribution were tested to estimate base hazard. After the base hazard was identified, FVIII activity effect was evaluated as a proportional hazard effect on base hazard. FVIII activity was predicted using model-based individual pharmacokinetic parameters from a population pharmacokinetic (popPK) model (i.e. popPK model [A]) that used Phase 1 and 3 clinical data with efanesoctocog alfa. Potential covariate effects were tested on the base hazard using stepwise covariate modeling in PsN (v.5.2.6).

Following stepwise covariate modeling and model refinement, treatment effect (once-weekly prophylaxis and on-demand treatment) was selected as a covariate on base hazard. Treatment effect on base hazard showed that hazard (A) for a bleeding event was about 9-fold higher for on-demand treatment than for prophylaxis.

Among the various base and FVIII effect models evaluated, both Weibull function as base hazard, and power model for effect on FVIII activity provided the lowest statistically significant objective function value:

$$h ( t ) = λ {γ ( λ t )}^{γ - 1} {e}^{β {C}^{α}} {e}^{η} \qquad [ B ]$$

Model [B] Abbreviations are: h: hazard function of Weibull distribution; t: time; λ: constant of the base hazard; γ: shape parameter of the base hazard; β: constant of the FVIII effect; C: FVIII activity (drug plasma concentration); α: shape parameter of the FVIII effect; η: individual random effect.

The appropriateness of the model was assessed by comparing the percentage of patients without bleeding events observed in XTEND-1 versus that predicted by the RTTE model using Kaplan-Meier (KM) visual predictive checks, statistical significance of identified covariates, and other aspects such as precision of the parameter estimates. The parameter estimates with RSE (%) and 95% confidence interval (CI) from the SIR are shown in Table 2, confirming that the parameter estimates are contained within the 95% CI.

TABLE 2
Parameter estimates of final RTTE model with 95% CI using SIR

| | Run | | | | |
|---|---|---|---|---|---|
| | run213 | | | | |
| | OFV | | | | |
| | 5334.632 | | | | |
| | Condition number | | | | |
| | 81.9 | | | | |
| Parameter | Unit | Estimate | SIR 95% CI | RSE (%) | SHR (%) |
| Constant of the base hazard (λ) | hour⁻¹ | 0.00247 | 0.00112:0.00508 | 36 | — |
| Shape parameter of the base hazard (γ) | — | 1.02 | 0.900:1.16 | 6 | — |
| Constant of the FVIII effect (β) | (IU/dL)⁻¹ | −1.73 | −2.35:−1.24 | 15 | — |
| Shape parameter of the FVIII effect (α) | — | 0.247 | 0.191:0.311 | 11 | — |
| On-demand arm effect on the base hazard | — | 2.21 | 1.58:2.90 | 14 | — |
| IIV Base hazard | CV (%) | 114 | 87.3:147 | 13 | 33 |

For Table 2, abbreviations are OFV: objective function value;
SIR: sampling importance resampling,
CI: confidence interval;
RSE: relative standard error from NONMEM;
SHR: shrinkage on standard deviation scale;
CV: coefficient of variation

The parameter estimates from the final RTTE model [B] illustrate that the Weibull base hazard with λ of 0.00247 h¹ and shape parameter γ of 1.02, is slowly increasing with time since γ>1. For the power model of FVIII activity effect, the estimates of β=−1.73 (IU/dL)⁻¹ and α=0.247 shows that the hazard (hazard ratio [HR]=3.76) was 3.76-fold higher for FVIII activity of 1 IU/dL compared to FVIII activity of 10 IU/dL. The hazard for on-demand treatment was approximately 9.12-fold higher (HR=e2.21) than the hazard for prophylaxis treatment. Finally, the inclusion of treatment effect as covariate on base hazard decreased the IV on base hazard from % CV of 148% (run201) to % CV of 114% (run213).

Model Evaluation Results: Using the final RTTE model [B], the probability of being bleed-free in 1 year in a typical patient of 78.3 kg was simulated for once-weekly efanesoctocog alfa 50 IU/kg prophylaxis and for theoretical continuous infusion regimens (e.g., targeting stable levels of 10-20 IU/dL FVIII activity), using typical parameters of the RTTE model and parameter uncertainty. The annualized bleed rate was simulated for once-weekly efanesoctocog alfa 50 IU/kg prophylaxis using a virtual population of 1000 adult/adolescent patients for which the interindividual variability (IIV) in pharmacokinetics was incorporated using a popPK model, together with the IV from the RTTE model.

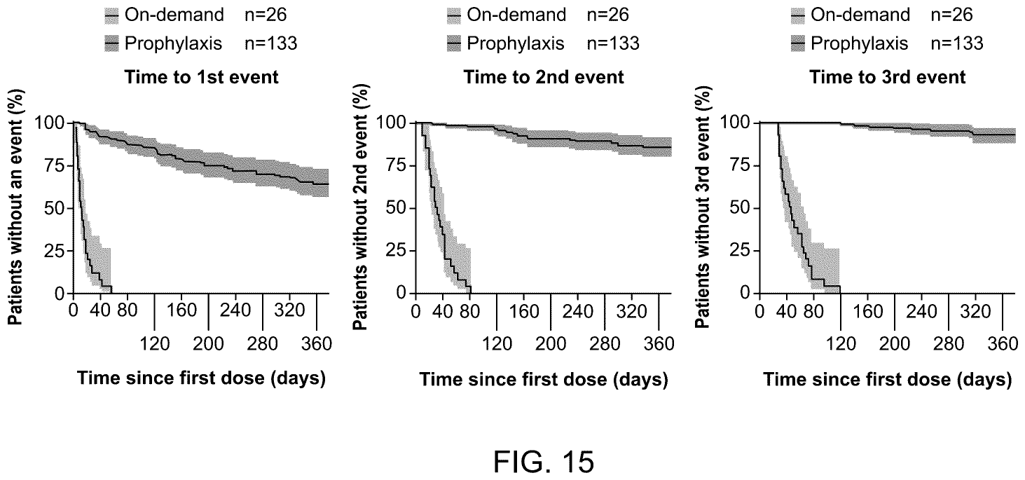

In the observed data, 362 bleeding events occurred during the study, 94 during prophylaxis (86 during the 52-week prophylaxis period in Arm A [n=133] and 8 during the 6-month prophylaxis period in Arm B [n=26]) and 268 during on-demand treatment (the initial 6-month period of Arm B [n=26]). One bleeding event occurred before the start of efanesoctocog alfa treatment and was not included in RTTE model development. The observed data shows a clear difference in the percentage of patients without bleeding events between on-demand treatment and prophylaxis arms. The percentage of patients without bleeding events is higher for prophylaxis than for on-demand treatment, and the time to a bleeding event is longer for prophylaxis versus on-demand (see FIG. 15).

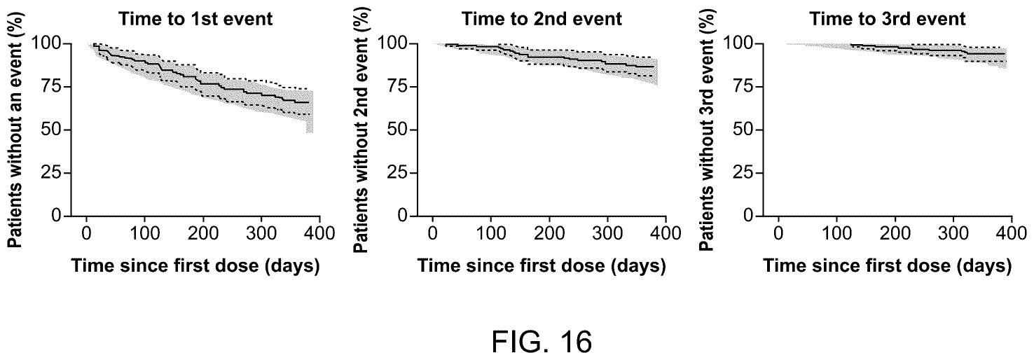

Kaplan-Meier (KM) visual predictions of the final RTTE model [B] for the prophylaxis arm in the XTEND-1 study are shown in FIG. 16. The solid line is observed KM and the dashed lines are 95% confidence interval around the observed KM. A grey band shows the visual predictive check with 95% prediction interval. RTTE, repeated time to event. The KM visual predictive check for the final model (see FIG. 16) provides a good description of the observed KM survival and demonstrated that the model was able to capture the general tendency of the data as well as their variability.

Annualized bleed rate (ABR) and bleed-related outcome simulations: Simulations were conducted with the final RTTE model to predict bleeding events in a virtual population of 1000 adult & adolescent patients on prophylaxis regimen of 50 IU/kg QW for 52 weeks. The individual simulated ABR was computed from simulations. The simulated mean (SD) ABR was 0.71 (1.50) and median ABR was 0 (Q1-Q3: 0-1). This was in good agreement with the observed ABR for the Arm A of XTEND-1 study in which the mean (SD) ABR was 0.71 (1.43) and median ABR was 0. (See Table 3). The similarity between simulated ABR and observed ABR shows that the final RTTE model is able to predict the ABR in a population treated with 50 IU/kg QW BIVV001 prophylactically.

64.1% virtual patients had 0 bleeds in the simulations, which was similar to the percentage in Arm A of the XTEND-1 study where 64.7% (86 out of 133) patients were observed to have 0 bleeds during the efficacy period. (See Table 3). Thus, the final RTTE model was also able to adequately predict the percentage of patients without bleeds in a population receiving 50 IU/kg QW efanesoctocog alfa prophylactically.

TABLE 3
Comparison of simulated (RTTE) and observed
(XTEND-1) bleed-related outcomes

| | Mean (SD) | Patients with no bleeding |
|---|---|---|
| | ABR | events (%) |
| Simulated ABR | 0.71 (1.5) | 64.1 |
| XTEND-1 prophylaxis arm | 0.71 (1.4) | 64.7 |

Probability of first bleed in 1 year simulation: The RTTE model simulation was used to determine the probability of being bleed-free in 1 year in a typical patient of 78.3 kg using different efanesoctocog alfa dosing regimens. Evaluated dose regimens of efanesoctocog alfa included 50 IU/kg QW efanesoctocog alfa (i.e. prophylaxis) and continuous infusion regimens (CIR) of 10, 12, and 15 IU/kg. In this simulation, CIR is infused over 1 week. For example, for 10 IU/kg continuous infusion in a 78.3 kg patient, 783 IU are infused over 1 week & continued for 52 weeks. RTTE model simulation results are shown in Table 4.

TABLE 4
Probability of being bleed-free in 1 year in a typical patient

| | Ctrough or stable | Time >10 IU/dL | Time >40 IU/dL | Probability of | |
|---|---|---|---|---|---|
| Dosing | FVIII at | FVIII | FVIII | being bleed-free | |
| Regimen | steady state | (% of dosing | (% of dosing | in 1 year | |
| (QW or CIR^{a}) | (IU/dL) | interval) | interval) | (%) (95% CI) | |
| 50 IU/kg QW | 12.8 | 100 | 52.6 | 71 | (50-83) |
| 10 IU/kg CIR | 10.8 | 97.9 | 0 | 36 | (16-56) |
| 12 IU/kg CIR | 12.9 | 98.8 | 0 | 42 | (20-60) |
| 15 IU/kg CIR | 16.1 | 99.2 | 0 | 48 | 26-65) |

CI, confidence interval; CIR, continuous infusion regimen; FVIII, factor VIII; QW, once weekly.
^{a}Continuous infusion is dose infused over 1 week, e.g., for 10 IU/kg continuous infusion, in a 78.3 kg patient, 783 IU are infused over 1 week & continued for 52 weeks.

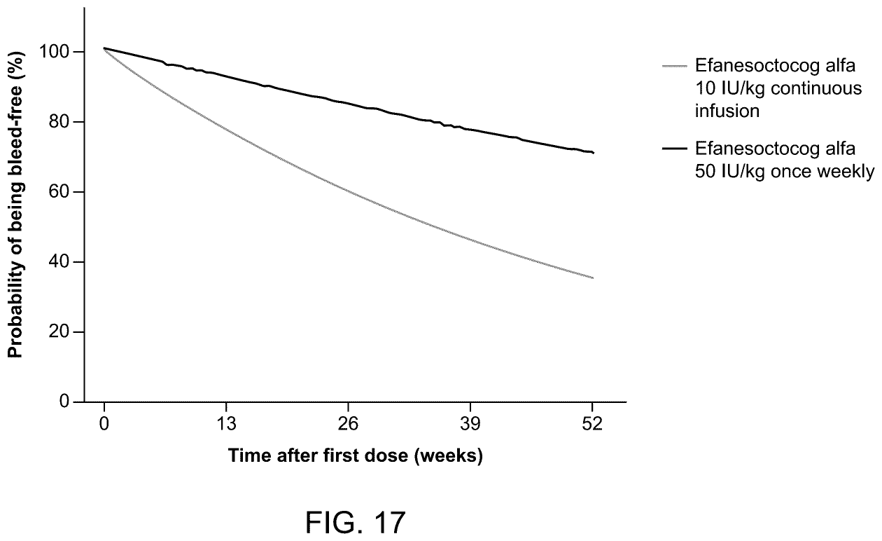

As shown in Table 4, the probability (%) of being bleed-free in 1 year was 71% (95% CI: 50%-83%) for a once-weekly efanesoctocog alfa 50 IU/kg prophylaxis regimen. The probability of being bleed-free with a once-weekly efanesoctocog alfa 50 IU/kg prophylaxis regimen was 23%-35% higher than a CIR of 10 IU/kg, which maintained stable FVIII activity in the range of 10.8-16.1 IU/dL. None of the CIR provided any significant time at >40 IU/dL FVIII during the dosing interval. The probability of being bleed-free with efanesoctocog alfa 50 IU/kg once-weekly prophylaxis and 10 IU/kg continuous infusion is shown in FIG. 17.

Conclusions: The bleeding events in adults & adolescents treated with IV efanesoctocog alfa were adequately characterized by the RTTE model [B] with a Weibull base hazard and effect of FVIII activity on base hazard modeled by a power model. Treatment effect (on-demand or prophylaxis) was identified as a statistically significant covariate on base hazard indicating that bleeding hazard was 9.12 fold higher for on-demand treatment relative to prophylaxis treatment.

Simulations in a virtual adult & adolescent population for a 50 IU/kg QW prophylaxis regimen showed the simulated ABR and percentage of patients with zero bleeds were comparable to that observed in Arm A of the XTEND-1 study. Simulations for a 50 IU/kg QW prophylaxis regimen in a typical patient predicted that the probability of first bleed (95% CI) in 1 year was 29% (17%-50%). The probability of first bleed in 1 year by was lowered by approximately 23% to 35% for a 50 IU/kg QW prophylaxis regimen compared to CIR than maintain stable FVIII activity in the range of 16.1 IU/dL to 10.8 IU/dL. As such, the RTTE model simulations of once-weekly 50 IU/kg efanesoctocog alfa prophylaxis estimated a 2-fold higher probability of being bleed-free in 1 year, compared with stable FVIII activity from 11-16 IU/dL achieved via CIR. These results further support the benefits of sustained normal to near-normal FVIII activity (>40 IU/dL) achieved by 50 IU/kg QW prophylaxis regimen on a hemophilia A subject.

### Example 2. Population Pharmacokinetic (popPK) Model to Characterize Efanesoctocog Alfa Factor VIII (FVIII) Activity Levels in Patients with Severe Hemophilia A

Once-weekly efanesoctocog alfa provides high sustained FVIII activity in the normal to near-normal range for most of the week and demonstrated superior bleed protection compared with prior FVIII prophylaxis. FVIII activity data have been collected from 5 clinical studies (Phase 1/2a single- and repeat-dose studies [NCT03205163 and EudraCT 2018-001535-51, respectively] in adults, and Phase 3 studies in adults and adolescents ≥12 years of age [XTEND-1, EFC16293, NCT04161495] and children 1 year to <12 years [XTEND-Kids, NCT04759131], and Phase 3 long-term extension study [XTEND-ed, NCT04644575]). A popPK model was developed to characterize FVIII activity after efanesoctocog alfa dosing, identify intrinsic and extrinsic factors affecting pharmacokinetics (PK), and assess PK variability.

FVIII activity levels used to develop the popPK model were measured by the one-stage clotting assay from 3054 blood samples from 199 adults and adolescents, and 61 children who received efanesoctocog alfa in the aforementioned studies. Body weight and VWF level ranged from 12.5 kg-133 kg and 40 IU/dL-339 IU/dL, respectively. A one-compartment model with linear elimination was used to characterize FVIII activity with an estimated allometric body weight effect on clearance (CL) and volume of central compartment (V) to account for the dependence of CL and V on body size. The efanesoctocog alfa popPK model is shown as equation [A], above.

Baseline VWF, baseline race, race (White and Asian), hepatitis C virus and human immunodeficiency virus status, and blood types (A, B, 0) were tested for statistical significance in the covariate analysis. The final popPK model was used to simulate various dose regimens in a virtual population of adult and adolescent patients generated using baseline body weight distribution from the Phase 1/2a studies and XTEND-1.

### Results:

TABLE 5
Parameter estimates of the final popPK
model for efanesoctocog alfa

| Parameter | Estimate | RSE (%) |
|---|---|---|
| Typical value of CL (⊖₁, dL/h) | 0.433 | 1.43 |
| Typical value of V (⊖₂, dL) | 30.2 | 1.04 |
| Power of coefficient of bodyweight on CL | 0.677 | 3.91 |
| Power of coefficient of bodyweight on V | 0.791 | 3.15 |
| Proportional coefficient of Asian Race on | 0.896 | 2.56 |
| CL | | |

| | Inter-individual variability | | |
|---|---|---|---|
| Parameter | Estimate (% CV) | RSE (%) | Shrinkage (%) |
| CL | 0.0354 (19.0) | 14.5 | 6.07 |
| V | 0.0209 (14.6) | 14.5 | 12.6 |
| Corr-CL-V | 0.0148 | 21.6 | |

Residual variability

| Parameter | Estimate | RSE (%) |
|---|---|---|
| Proportional term | 0.182 | 5.70 |
| Additive term (IU/dL) | 0.837 | 39.4 |

Table 5 Abbreviations are: CL, clearance from central compartment; CV, coefficient of variation; popPK, population pharmacokinetic; RSE (%), percentage of relative standard error (SE) (100% × SE/estimate); V, volume of central compartment; ⊖ is the population estimate of PK parameter.

The final popPK model described the FVIII activity over time profile, captured inter-individual variability in FVIII activity, and precisely estimated moderate inter-individual variability in CL and V (Table 5). Body weight effect allometric exponents showed that CL and V increase with body weight, with overall faster elimination with lower body weight. Asian race was identified as a statistically significant covariate on CL (P<0.001); CL in Asians was 10.4% lower than in non-Asians. Baseline VWF level was not identified as a statistically significant covariate in the final popPK model, consistent with prior studies that demonstrate that the PK of efanesoctocog alfa is VWF-independent. Blood type was not identified as a statistically significant covariate in the final popPK model.

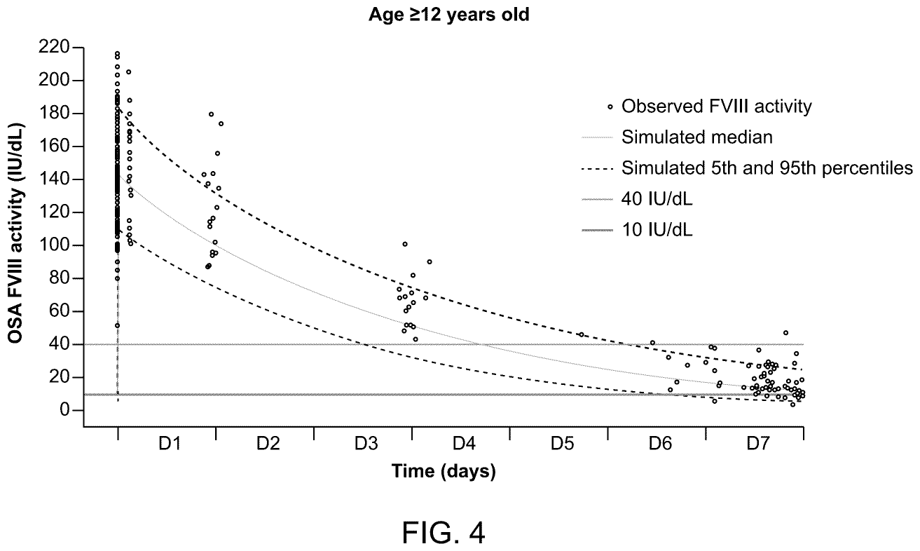

Simulated steady-state FVIII activity over time for efanesoctocog alfa and population predicted and individual predicted versus observed FVIIII activity in the final popPK model is presented in FIG. 4, which illustrated FVIII activity >40 IU/dL for 3 to 4 days post dose. The final popPK model showed that a once-weekly efanesoctocog alfa (50 IU/kg) prophylaxis regimen achieves a steady state C_{trough} of >10 IU/dL and the time to 40 IU/dL FVIII activity was 3 to 4 days in the majority of adult and adolescent patients, irrespective of body weight and race. Simulations for perioperative management during major surgery and treatment of major bleeds showed that a loading dose of 50 IU/kg, followed by 30 IU/kg every 3 days in the postoperative period, met the World Federation of Hemophilia guidelines for peak FVIII activity (>50 IU/dL to 80 IU/dL) for most adults and adolescents. Similarly, for minor surgeries and treatment of moderate to minor bleeds, a single dose of 50 IU/kg efanesoctocog alfa resulted in peak FVIII activity that met these guidelines.

Conclusions: A linear one-compartment popPK model was able to adequately characterize FVIII activity in patients with severe hemophilia A. Although CL and V depended on body weight and Asian race was identified as a covariate on CL, body weight and Asian race's limited influence on FVIII exposure was not considered clinically meaningful. PopPK simulations demonstrated that 50 IU/kg once weekly efanesoctocog alfa achieved sustained FVIII activity in the normal to near-normal range (>40 IU/dL) for 3-4 days and >10 IU/dL at Day 7 in most adults and adolescents. PopPK simulations also supported the Phase 3 dose regimens selected for regular prophylaxis, treatment of bleeds, and perioperative management. Efanesoctocog alfa individual clearance was independent of baseline VWF in adults and adolescents.

Additional Details Relating to the Development of the popPK Model

Available data from adult, adolescent, and pediatric phase 3 studies with efanesoctocog alfa were incorporated in development of the population pharmacokinetic (popPK) model. Complete data from adult and adolescent study and partial data from pediatric and long-term safety studies were also included.

TABLE 6
Description of studies used in the popPK analysis

| | | BIVV001 | | | |
|---|---|---|---|---|---|
| | | Dose | Duration of | | |
| Phase | Study | Regimens | Treatment | Population | N^{a} |
| 1/2^{a} | TDU16220 | IV 25 and | Single dose | Adult (Age >18 year) Hemophilia A | 15 |
| | | 65 IU/kg | | patients | |
| 1/2^{a} | TDR16219 | IV 50 and | Multiple dose, | Adult (Age >18 year) Hemophilia A | 24 |
| | | 65 IU/kg | QW for 4 weeks | patients | |
| 3 | EFC16293 | IV 50 IU/kg | Multiple dose, | Adult and adolescent (Age ≥12 year) | 159 |
| | | | QW for 52 weeks | Hemophilia A patients | |
| 3 | EFC16295 | IV 50 IU/kg | Multiple dose, | Pediatric (Age <12 year) Hemophilia | 74 |
| | | | QW for 52 weeks | A patients | |
| 3 | LTS16294 | IV 50 IU/kg | Multiple dose, | Pediatric, adult and adolescent | 205^{b} |
| | | | QW | Hemophilia A patients | |

^{a}Number of patients exposed to BIVV001 in each study
^{b}161 patients from Arm A (rolled over from EFC16293 or EFC16295), 37 patients from Arm B and 7 patients from Arm C of the LTS16294 study are included in the PopPK model assessment.

Table 6 Definitions: a) Number exposed to efanesoctocog alfa in each study for popPK model development; total N=260, with 199 adults & adolescent patients and 61 pediatric patients from EFC15295; b) Only patients who have surgery in (3 patients) the LTS16294 study are included in the popPK model development; c) In EFC16293, 17 patients are in sequential arm, in which patients skip the dose on week 1 day 7 and week 26 day 7, to allow estimation of terminal half-life by collection of one stage clotting (OSC) FVIII activity samples up to day 15 after day 1 and week 26 dose.

Phase 1 popPK analysis described the OSC FVIII activity data using 1-CMT model with bodyweight as a covariate on CL, V and level of hematocrit as covariate on V. In the final popPK analysis, the 1-CMT model is chosen to be the structural model to describe OSC FVIII activity profile, and the base model includes WT effect. Further covariate screening was done on base POP PK model.

Bodyweight in kg (WT), or other continuous covariates, was scaled to median baseline WT (median baseline value) in adults and adolescents (78.3 kg) for evaluating as adult covariate effect or pediatric allometry effect.

For example, CL=TVCL*(WT_{time-varying}/78.3)^{CLexp}*(exp(ETA1)), for adult covariate effect and pediatric allometry effect. A similar approach was used for volume.

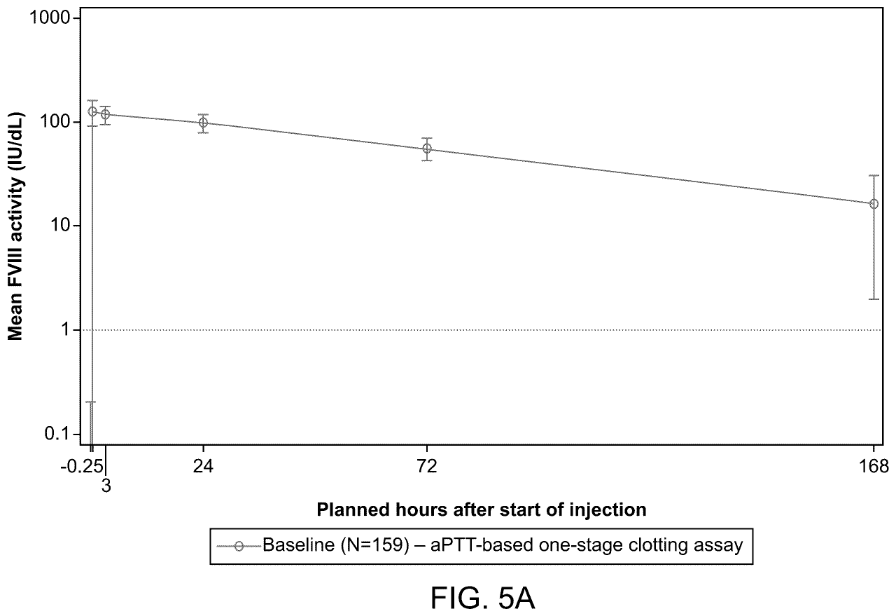

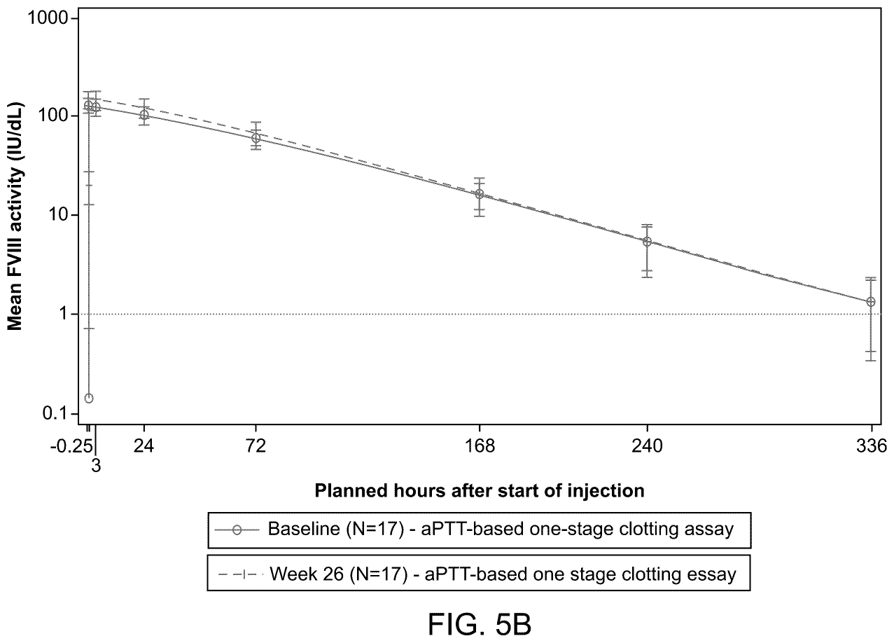

Datasets: One popPK dataset for observed data is based on the adult/adolescent study EFC16293. FIG. 5 shows a baseline corrected FVIII activity time profile. Day 1 (baseline) is shown for all patients (FIG. 5A). Day 1 (baseline) and at Week 26 is shown for sequential arm patients (FIG. 5B). The FVIII activity time profile follows a general one-compartment (linear decline on log-scale) type kinetics. The FVIII activity shows a mean half life for efanesoctocog alfa at 47.8 hours.

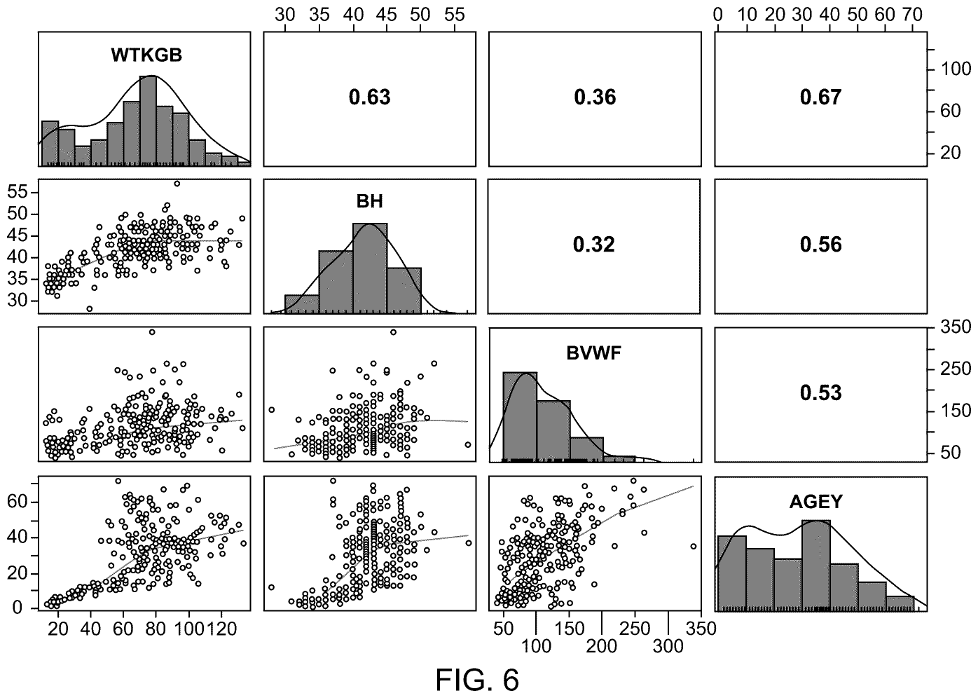

The correlation between 4 continuous covariates at baseline is shown in FIG. 6. Baseline weight (WTKGB) had a median of 78.3, which excluded the EFC16295 study. Baseline race (BH) had a median of 43 (also excluding the EFC16295 study). Baseline VWF (BVWF) showed a median of 112 (also excluding the EFC16295 study). Age appeared to be correlated to WT, so age is not tested as a covariate. Race and VWF were tested as covariates and found not to be significant.

The distribution of categorical covariates such as blood type, race, HIV status, and HCV status is shown in Table 7. Data in table 7 includes all studies (n=260). Black (1.92%) and Other race (3.08%) are present in about 5% of patients. For blood types, blood type A & O have more patients, 29.62% and 36.15% respectively. Blood type B is present in <10% (9.23%) of patients. Blood type AB is present in <5% (3.85%) of patients. HCV and HIV positive patients are older with no pediatric patients being HCV or HIV positive. There are 2 patients with Age<2 years. Out of the categorical factors shown, only HCV status, HIV status, blood type (A, B and O), and Race (Caucasian and Asian) were tested as covariates.

TABLE 7
Descriptive statistics of the catgorical covariates

| STUDY | N | COVARIATE | TYPE | n (%) | |
|---|---|---|---|---|---|
| All | 261 | GENDER | Female | 1 | (0.38) |
| All | 261 | | Male | 260 | (99.62) |
| All | 261 | Age group | Age >= 18 | 173 | (66.28) |
| All | 261 | | 12 <= Age < 18 | 26 | (9.96) |
| All | 261 | | 6 <= Age < 12 | 35 | (13.41) |
| All | 261 | | Age < 6 | 27 | (10.34) |
| All | 261 | Hepatitis C | With | 76 | (29.12) |
| All | 261 | infection status | Without | 185 | (70.88) |
| All | 261 | HIV infection | With | 24 | (9.2) |
| All | 261 | status | Without | 237 | (90.8) |
| All | 261 | RACE | Caucasian | 178 | (68.2) |
| All | 261 | | Asian | 40 | (15.33) |
| All | 261 | | Black | 5 | (1.92) |
| All | 261 | | Other race | 8 | (3.07) |
| All | 261 | | Race not reported | 30 | (11.49) |
| All | 261 | BLOOD TYPE | Blood type A | 77 | (29.5) |
| All | 261 | | Blood type AB | 10 | (3.83) |
| All | 261 | | Blood type B | 24 | (9.2) |
| All | 261 | | Blood type O | 94 | (36.02) |
| All | 261 | | Blood type unknown | 56 | (21.46) |

Table 8 provides additional details regarding the covariate model.

TABLE 8
Parameter estimates of the final popPK
model for efanesoctocog alfa

| Parameter | Estimate | RSE (%) |
|---|---|---|
| Typical value of CL (⊖₁, dL/h) | 0.433 | 1.43 |
| Typical value of V (⊖₂, dL) | 30.2 | 1.04 |
| Power of coefficient of body weight on CL | 0.677 | 3.91 |
| Power of coefficient of body weight on V | 0.791 | 3.15 |
| Proportional coefficient of Asian race on | 0.896 | 2.56 |
| CL | | |

| | Inter-individual variability | | |
|---|---|---|---|
| Parameter | Estimate (% CV) | RSE (%) | Shrinkage (%) |
| CL | 0.0354 (19.0) | 14.5 | 6.07 |
| V | 0.0209 (14.6) | 14.5 | 12.6 |
| Corr-CL-V | 0.0148 | 21.6 | |

Residual variability

| Parameter | Estimate | RSE (%) |
|---|---|---|
| Proportional term | 0.182 | 5.70 |
| Additive term (IU/dL) | 0.837 | 39.4 |

OSC activity is concentration (C) in central compartment. All parameters for base and final covariate model were estimated with acceptable precision. Adding bodyweight effect decreased the instrumental variables estimation (IIV) on CL and V, while adding the Asian race effect on CL decreased the IV on CL. The exponents for WT effect on CL and V are acceptable when compared to the simple allometry exponents. Asian race effect was identified on CL, with clearance for Asians 10.4% lower than non-Asians of identical bodyweight.

The PK parameters incorporated with covariates in the final popPK model are shown below:

$$CL = 0.433 {( \frac{Bodyweight ( kg )}{78.3} )}^{0.677} {( 0.896 )}^{Asian} {e}^{{η}_{1}} \qquad [ {A}^{′} ]$$

$$V = 30.2 {( \frac{Bodyweight ( kg )}{78.3} )}^{0.791} {e}^{{η}_{2}}$$

Where Asian=1 for Asians and Asian=0 for non-Asians.

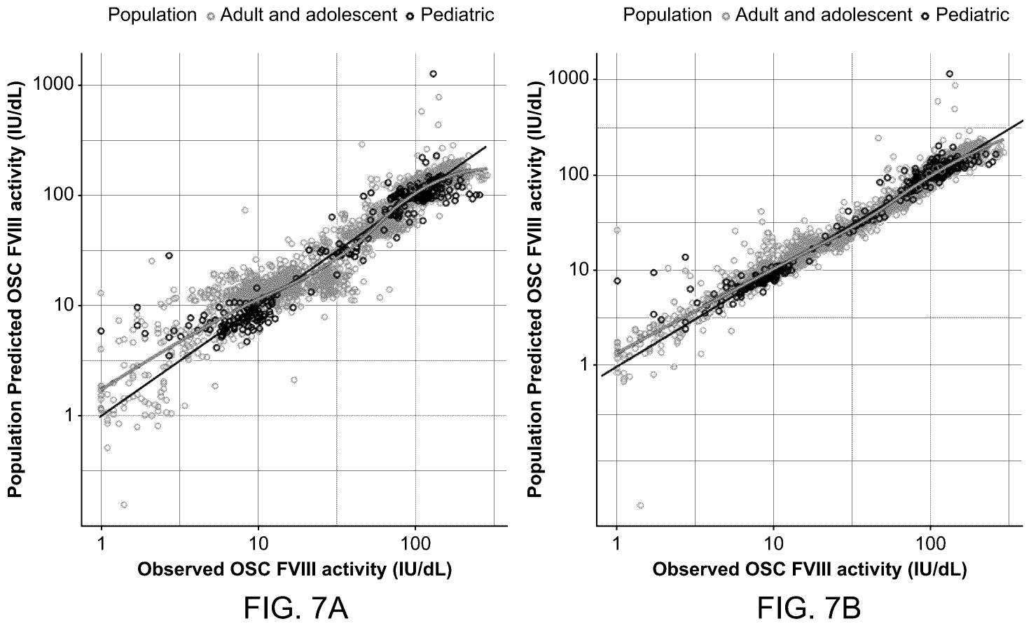

FIGS. 7A and 7B show population predictions (PRED) and individual predictions (IPRED) versus DV, respectively. This demonstrates that the population model and individual model are able to describe the PK data across the age categories.

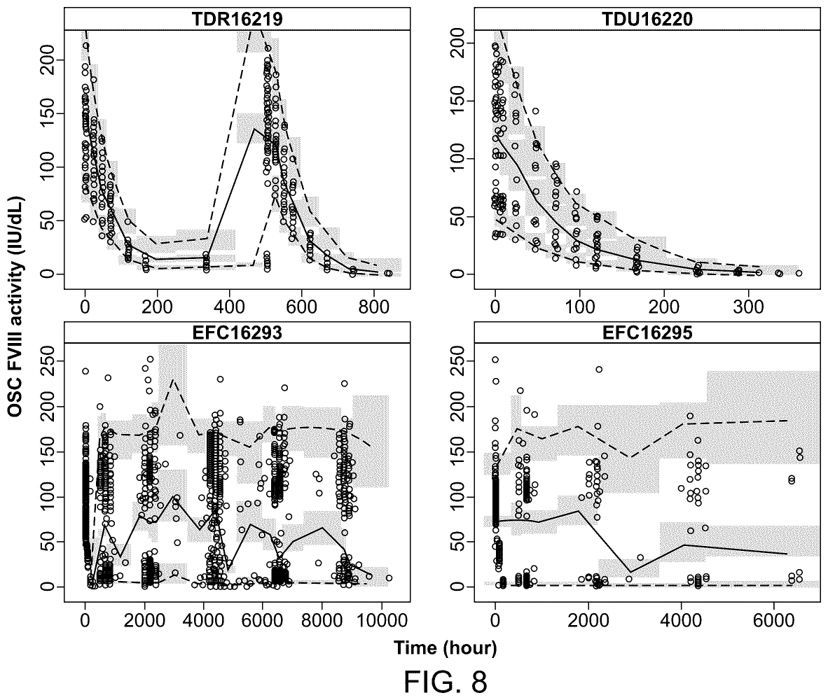

FIG. 8 demonstrates visual predictive checks (VPC) for the final popPK model. The VPC for each study show that a large majority of the observed FVIII activity data were within in the prediction range [5th-95th percentiles]. For the purpose of VPC, the one unique patient from LTS16294 was considered in EFC16293.

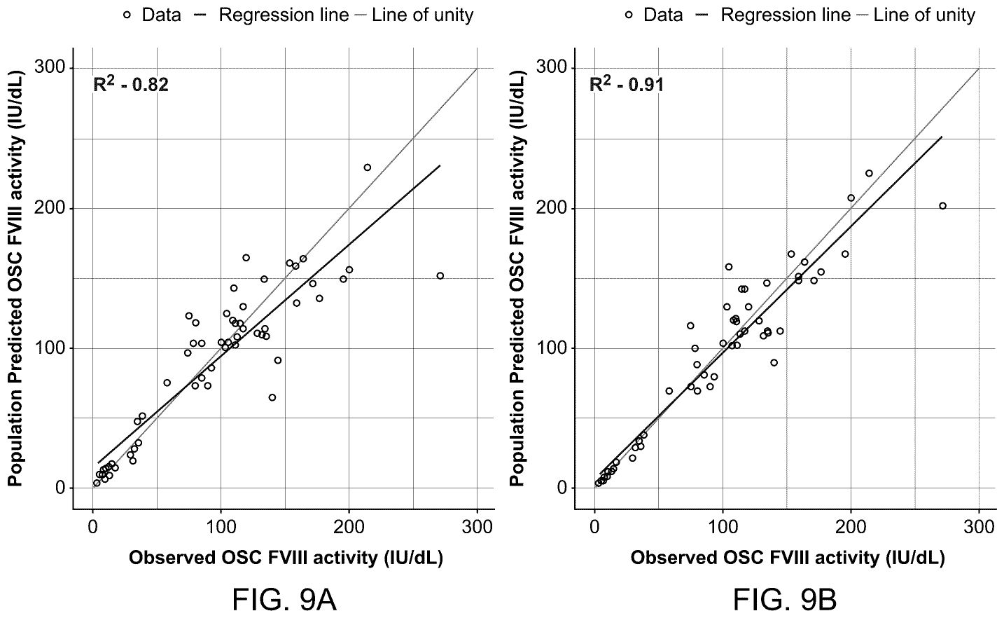

FIGS. 9A and 9B demonstrate population predictions (PRED) and individual predictions (IPRED) versus DV, respectively, for surgery. Data from 19 patients from EFC16293, EFC16295, and LTS16294 is included, during the surgery time frame. The model performs reasonably well in describing PK data collected during surgery and after adhoc surgery dosing.

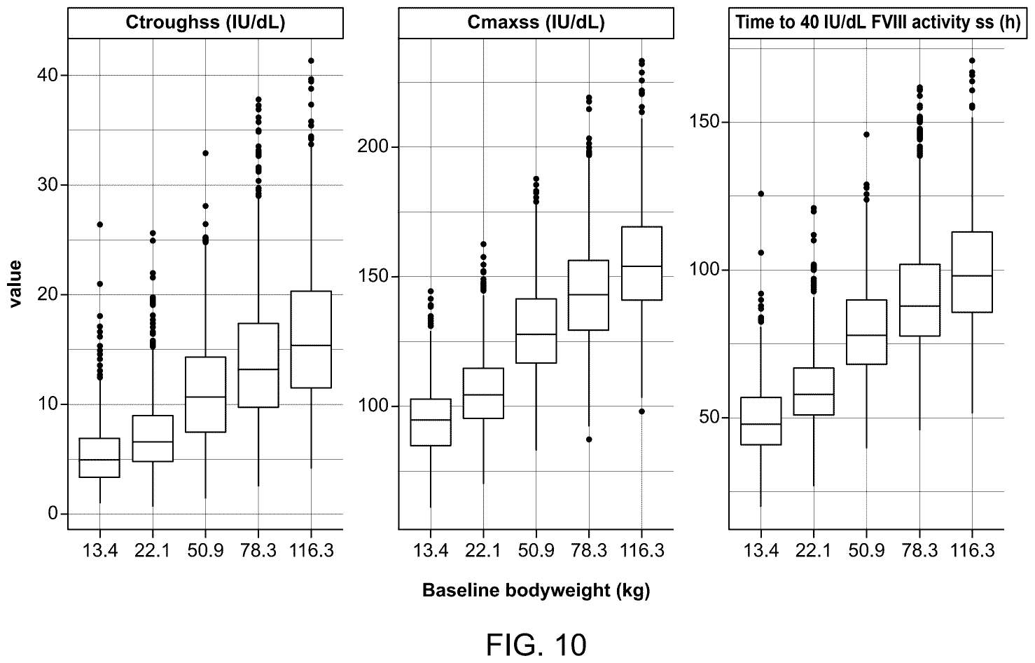

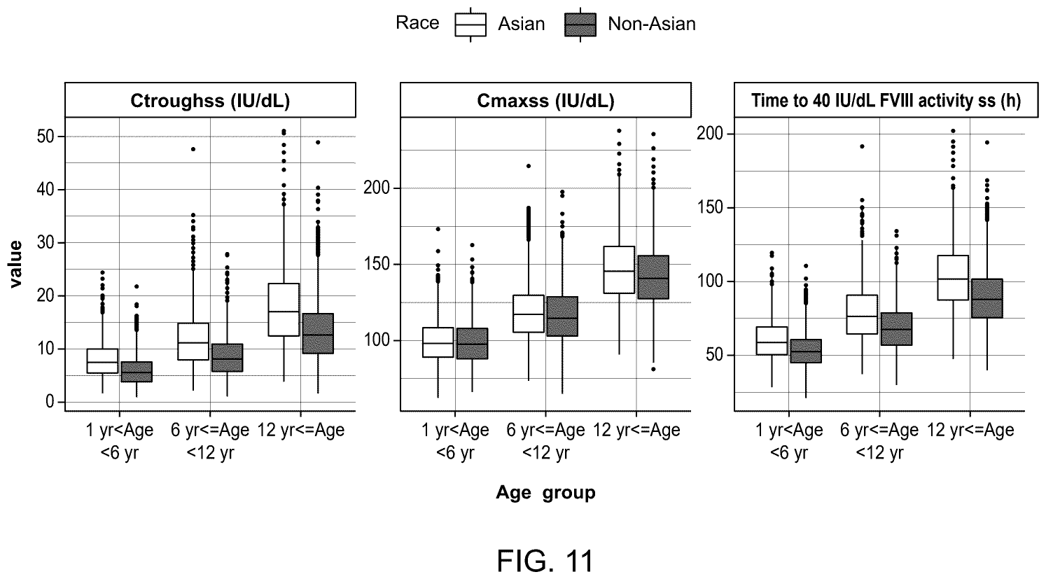

FIG. 10 shows the distribution of steady state C_{trough}, Cₘₐₓₛₛ and time to 40 IU/dL FVIII activity across all populations according to baseline body weight (kg). FIG. 11 shows the distribution of steady state C_{trough}, Cₘₐₓₛₛ and time to 40 IU/dL across non-Asian and Asian populations for all age groups. The steady state FVIII activity Cₘₐₓ, C_{trough} and time to 40 IU/dL FVIII activity increases with increasing body weight and is higher in Asians compared to Non-Asians. However, regardless of bodyweight and race, the 50 IU/kg QW prophylaxis regimen showed that a steady state C_{trough}>10 IU/dL and time to 40 IU/dL FVIII activity of 3 to 4 days is achieved for the majority of the adult and adolescent (Age ≥12 yr) population. The 50 IU/kg QW prophylaxis regimen also showed that a steady state C_{trough}>5 IU/dL & Time to 40 IU/dL FVIII activity of 2 to 3 days is achieved for the majority of the pediatric (Age <12 yr) population.

Major surgeries and major bleeds: The model was analyzed with regard to major surgeries and major bleeds. Major surgeries and major bleeds were categorized based on the criteria listed in Table 9.

TABLE 9

| Day | Major Surgery | Major Bleed |
|---|---|---|
| Pre-operative | Peak >80 IU-100 IU/dL | Peak >80 IU-100 IU/dL |
| (Day of bleed) | | |
| Day 1 to day 3 | Peak >60 IU-80 IU/dL | Peak >50 IU/dL (day 3) |
| Day 4 to day 7 | Peak >40 IU-60 IU/dL | Peak >50 IU/dL |
| Day 7 to day 14 | Peak >30 IU-50 IU/dL | Peak >50 IU/dL |

For major surgeries and bleeds, simulation was based on a dosing regimen of a single dose at 50 IU/kg with additional doses of 30 or 50 IU/kg every 2 to 3 days if needed. Thus, 50 IU/kg Q2D, 50 IU/kg Q3D, 30 IU/kg Q2D and 30 IU/kg Q3D are possible combinations of a dosing regimen after the pre-operative dose of 50 IU/kg (QXD is every X days). These same simulations as can be applied to both major surgeries and major bleeds, as both comprise the same dosing combinations.

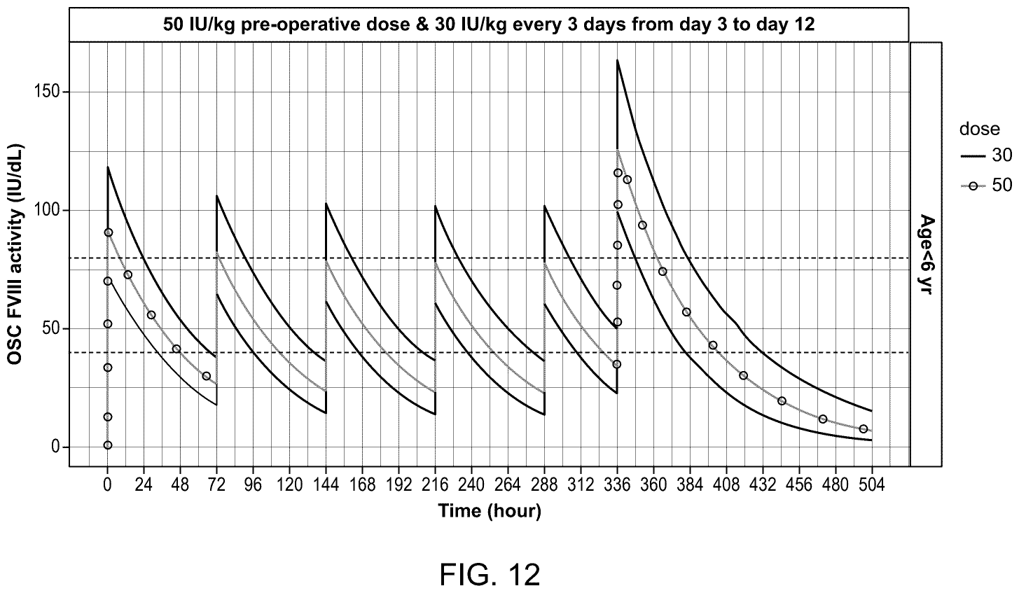

FIG. 12 shows the simulated OSC FVIII activity for major bleeds and major surgeries over time in subjects below six years of age. Across the entire surgery period, more than 95% of patients ages 6 and above meet the major surgery criteria, and more than 80% of patients under the age of 6 meet the surgery criteria, across the entire surgery period.

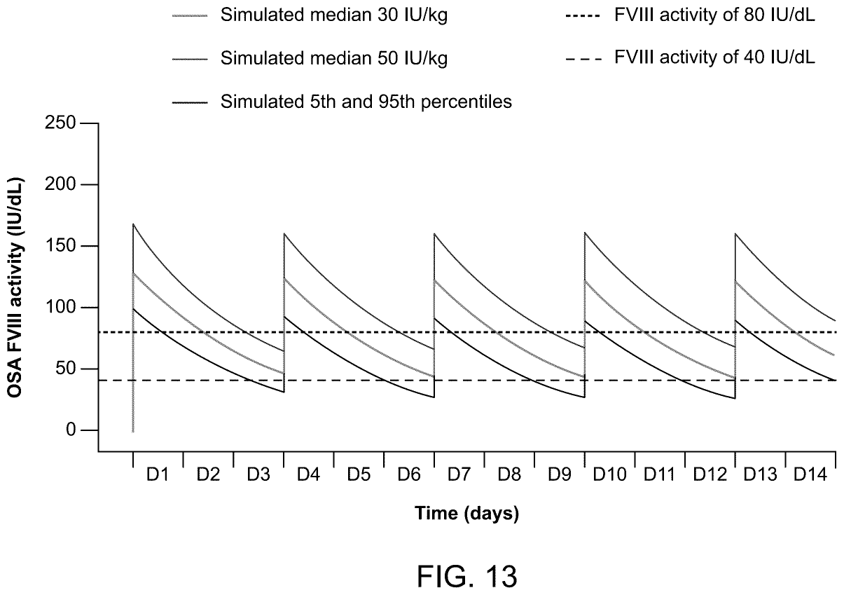

FIG. 13 shows the simulated FVIII activity for a dose of 50 IU/kg efanesoctocog alfa followed by 30 IU/kg every 3 days until Day 14 in a virtual adult and adolescent population. The simulations illustrated that an initial dose of 50 IU/kg, followed by 30 IU/kg every 3 days until Day 14, should be sufficient for perioperative management during major surgery, as well as for treatment of major bleeds. >95% of adult and adolescent patients are predicted to meet the World Federation of Hemophilia (WFH) guidelines (Srivastava A, et al. Haemophilia. 2020; 26 Suppl 6:1-158) for peak FVIII activity (>80-100 IU/dL preoperative or on day of major bleed). In addition, other dosing regimens such as an initial dose of 50 IU/kg followed by 50 or 30 IU/kg every 2 or 3 days were simulated, and these additional dosing regimens were also predicted to meet the WFH peak FVIII guidelines. The simulations thus illustrated that an initial dose of 50 IU/kg, followed by 50 IU/kg or 30 IU/kg every 2 or 3 days, would meet the WFH peak FVIII guidelines in the majority (>95%) of adult and adolescent patients to manage during major surgery and treatment of major bleeds.

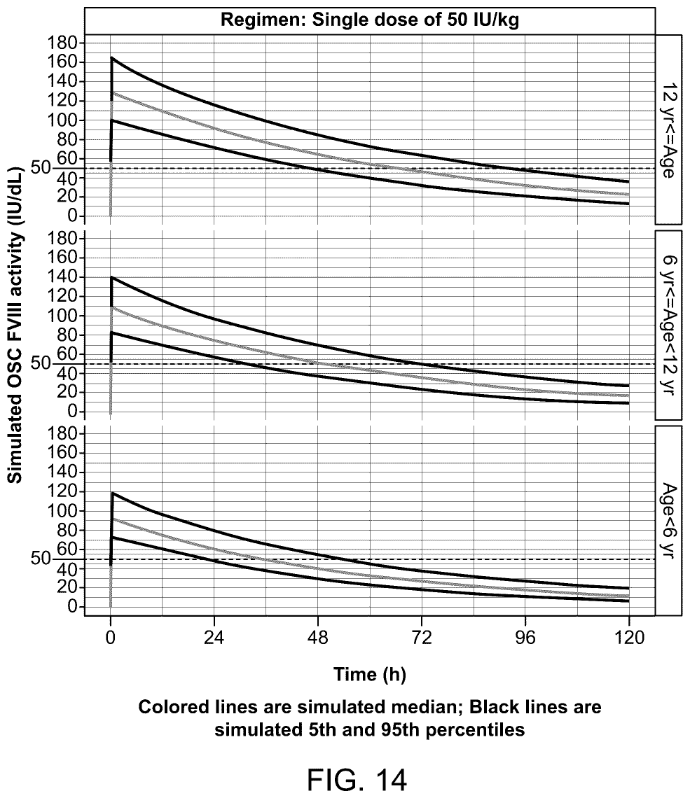

Minor surgeries and minor or moderate bleeds: FIG. 14 shows the simulated OSC FVIII activity overtime for all age groups. More than 95% of patients in all age groups meet the criteria of peak FVIII >50 IU/dL after the pre-operative dose for minor surgery. Similarly, more than 95% patients in all age groups meet the criteria of peak FVIII >40 IU/dL as needed for minor/moderate bleed management. With additional doses of 30 or 50 IU/kg every 2 or 3 days, more than 95% patients in all age groups meet the criteria of peak FVIII >50 IU/dL.

Conclusions: Based on this data, the one compartment (1-CMT) model describes the adult, adolescent, and pediatric OSC FVIII activity data reasonably well. Body weight effect (on CL and V) was included in the base model while Asian race effect (on CL) was identified as a statistically significant covariate. Simulations for a range of body weights show that fixed regimen of 50 IU/kg QW provides high FVIII activity in adult, adolescents, and pediatric populations, regardless of body weight and race. Simulations using this model were also able to support and show potential efanesoctocog alfa dosing schemes for surgery and bleeding scenarios.

### Example 3. Repeated Time to Event (RTTE) Analysis of Efanesoctocog Alfa in Pediatric Patients with Severe Hemophilia A

The XTEND-KIDS (EFC16295, NCT04759131) study was a Phase 3, open-label, multicenter study of the safety, efficacy, and pharmacokinetics efanesoctocog alfa in previously treated pediatric patients <12 years of age with severe hemophilia A. This example contains a description of the RTTE (bleeding) analysis of efanesoctocog alfa in pediatric patients with severe hemophilia A, aged <6 years and 6 to <12 years from the EFC16295 study. All pediatric bleed events were used to assess the model performances of a previously developed RTTE model in adult/adolescent patients (Model [B], see Example 1). As discussed above, this previous RTTE model was characterized by a Weibull baseline hazard from which the effect of OSC FVIII activity on baseline hazard was modeled with a power relationship.

The current analysis involved 1) an evaluation of the adult/adolescent RTTE model using the pediatric data to assess its ability in predicting the observed annualized bleed rates (ABRs) in the study EFC16295 and 2) the pediatric RTTE model was established by re-estimation of the model parameters using only the pediatric data. The pediatric RTTE model was used to compare observed and simulated ABRs in the pediatric population. Potential covariate-parameter relationships were also evaluated to assess if additional factors may affect the bleeding risk and ultimately the bleeding event rates in the pediatric population compared to adults and adolescents.

### Data:

Bleeding event data from 74 patients from the EFC16295 study was analyzed. This group of patients comprises the “pediatric BIVV001 RTTE analysis data set”. Key characteristics of the pediatric BIVV001 RTTE analysis data set are provided in Table 10. Overall, there were 63 bleed events recorded for all patients, 17 from the patients <6 years of age and 46 from the patients 6 to <12 years of age.

TABLE 10
Key characteristics of the pediatric BIVV001 RTTE analysis data set

| | <6 years | | 6 to <12 years | | Overall | |
|---|---|---|---|---|---|---|
| Number of patients | 38 | | 36 | | 74 | |
| Number of bleed events | 17 | | 46 | | 63 | |
| Age (year) | 4 | (1.4, 5) | 8 | (6, 11) | 5 | (1.4, 11) |
| [median (min, max)] | | | | | | |
| Weight (kg) [median | 18.0 | (11.4, 25.7) | 32.8 | (17.2, 66.5) | 22.0 | (11.4, 66.5) |
| (min, max)] | | | | | | |

| | |
|---|---|
| Development phase | III |
| Subject type | pediatric hemophilia A patients |
| Dose levels and regimens | BIVV001 50 IU/kg QW |

Of the 63 bleed events, 11 (17.5%) events were spontaneous bleed events, 30 (47.6%) events were traumatic bleed events, and 22 (34.9%) events were of unknown type. From the 63 bleed events, 42 (66.7%) events were joint bleed events, and 21 (33.3%) events were non-joint bleed events.

Patient baseline characteristics for the pediatric BIVV001 RTTE analysis data set are presented in Table 11, by age category and overall. The baseline bodyweight (WT) ranged from 11.4 to 66.5 kg with a median bodyweight of 22 kg. The age of patients in the dataset ranged from 1.4 years to 11 years with a median of 5 years.

The age at first prophylaxis ranged from 0 year (receiving prophylaxis right from first year of birth) to 5 years with a median of 1 year. This shows that 50% of patients had started receiving some type of FVIII prophylaxis treatment from a very early age, which resulted in a much-skewed distribution. Similarly, the distribution of number of target joints was also highly skewed as 72/74 (97%) patients had recorded 0 target joints (no target joint of bleeding concern). The hemophilia joint health score (HJHS) ranged from 0 to 24 with a median of 0. Baseline values were used for all covariates. Baseline was defined as the last available measurement before the first administration of study medication.

TABLE 11
Baseline characteristics of the patients in the pediatric
BIVV001 RTTE analysis data set: continuous covariates.

| | <6 years | | 6 to <12 years | | Overall | |
|---|---|---|---|---|---|---|
| | N = 38 | | N = 36 | | N = 74 | |
| Body weight (kg) | | | | | | |
| Mean (SD) | 17.9 | (3.53) | 35.8 | (12.9) | 26.6 | (12.9) |
| Median (min, max) | 18.0 | (11.4, 25.7) | 32.8 | (17.2, 66.5) | 22.0 | (11.4, 66.5) |
| Age (year) | | | | | | |
| Mean (SD) | 3.69 | (1.21) | 8.42 | (2.08) | 5.99 | (2.91) |
| Median (min, max) | 4.00 | (1.40, 5.00) | 8.00 | (6.00, 11.0) | 5.00 | (1.40, 11.0) |
| Age at 1st prophylaxis (years) | | | | | | |
| Mean (SD) | 0.789 | (0.875) | 1.22 | (1.15) | 1.00 | (1.03) |
| Median (min, max) | 1 | (0, 4) | 1 | (0, 5) | 1 | (0, 5) |
| Number of target joints | | | | | | |
| Mean (SD) | 0.0263 | (0.162) | 0.0556 | (0.333) | 0.0405 | (0.259) |
| Median (min, max) | 0 | (0, 1) | 0 | (0, 2) | 0 | (0, 2) |
| Number of bleeds 12 months prior | | | | | | |
| BIVV001 treatment | | | | | | |
| Mean (SD) | 1.97 | (2.49) | 2.08 | (5.29) | 2.03 | (4.07) |
| Median (min, max) | 1 | (0, 11) | 1 | (0, 32) | 1 | (0, 32) |
| Haemophilia Joint Health Score | | | | | | |
| Mean (SD) | 1.26 | (5.21) | 2.03 | (4.48) | 1.64 | (4.85) |
| Median (min, max) | 0 | (0, 24) | 0 | (0, 24) | 0 | (0, 24) |

Number of target joints: as assessed by the Investigator. A target joint is defined as a major joint (eg, hip, elbow, wrist, shoulder, knee or ankle) into which ≥3 spontaneous bleeding episodes occurred in a consecutive 6-month period. Number of bleeds 12 months prior BIVV001 treatment, referred to also as bleeding history in the current analysis. Hemophilia Joint Health Score range: 0-124, higher score indicates worse joint health.

### Covariate Analysis:

A search for significant covariate-parameter relationships was performed on the fixed adult/adolescent RTTE model to investigate if additional factors explain potential differences in bleeding risk in pediatric vs adult/adolescent patients. Potential covariate-parameter relationships were evaluated using the stepwise covariate model building procedure (SCM) with adaptive scope reduction (ASR) on the model with fixed adult/adolescent parameters to assess the effect of other factors (e.g. age, bleeding history) on bleeding risk in the pediatric patients. Of note, the covariate from the adult/adolescent RTTE model (on-demand treatment arm) is not relevant for the current analysis. Efanesoctocog alfa was administered only as prophylaxis treatment in the pediatric study EFC16295.

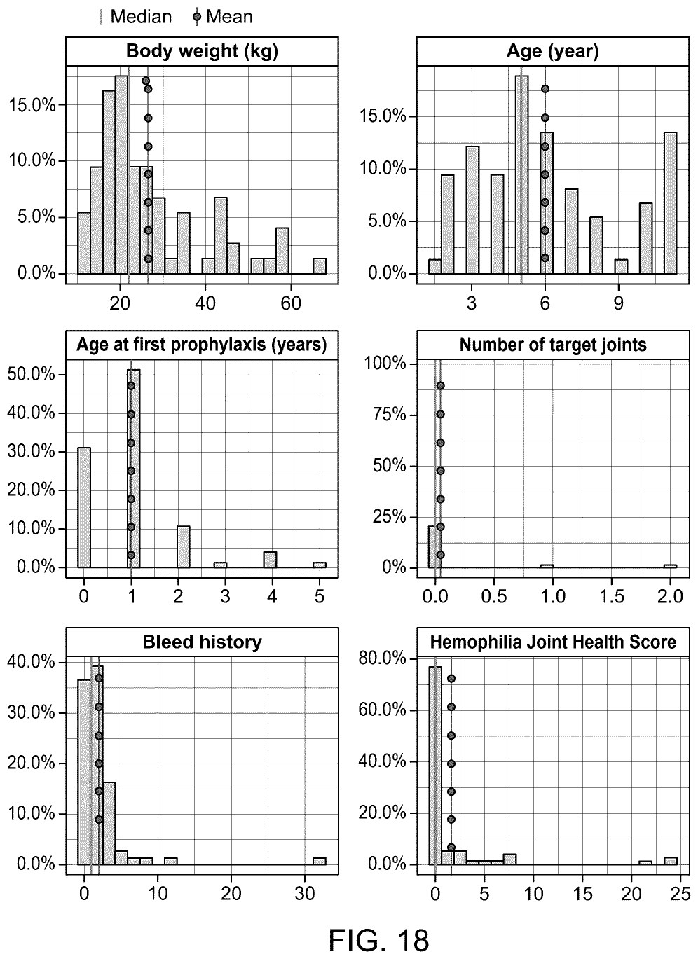

Histograms of covariates at baseline (FIG. 18) for the patients in the pediatric BIVV001 RTTE analysis data set show signs of skewed distribution of some disease history factors, such as age at first prophylaxis, number of target joints, bleed history in the 12 months prior to BIVV001 treatment and HJHS.

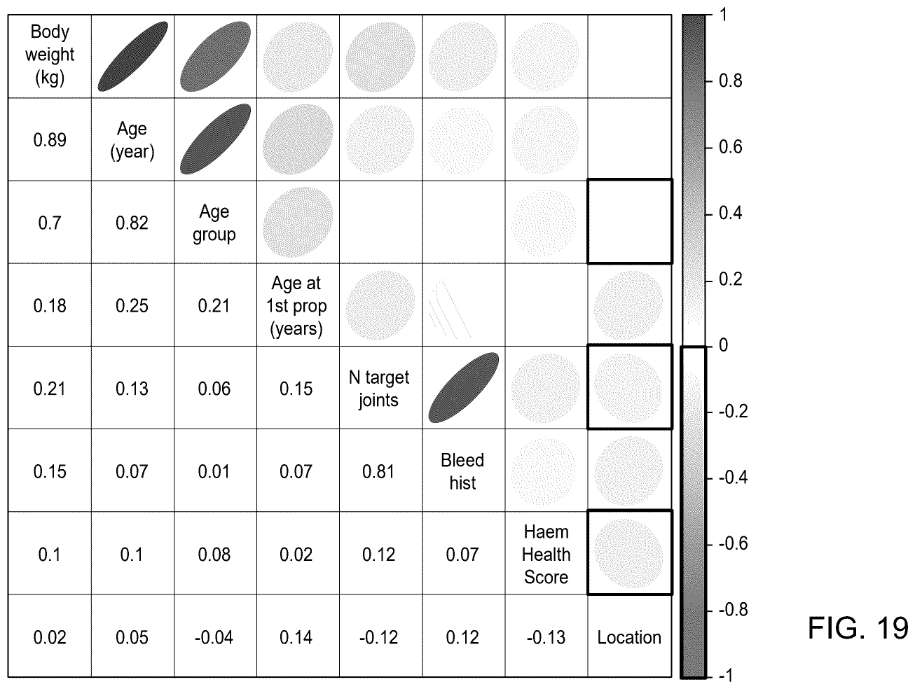

To assess covariate correlation, a correlation matrix of the covariates at baseline for the patients in the pediatric BIVV001 RTTE analysis data set is presented in FIG. 19. As expected, a strong correlation was observed for body weight (WT) and both age and age group (i.e. age continuous and age categorical). A strong correlation (>0.8) was also observed between the number of target joints and bleeding history. For covariates having a correlation coefficient >0.7, only age categorical (<6 years; 6 to <12 years) and bleeding history were included in the covariate model analysis.

The search for significant covariate-parameter relationships was performed on the RTTE model with fixed parameters to the adult/adolescent estimates using the SCM procedure to assess if other factors could account for the observed differences, but no significant covariate-parameter relationships were identified.

### RTTE Model Re-Estimation

To potentially improve the fit of the previous RTTE model to the pediatric population, the previous RTTE model was re-estimated on the pediatric efanesoctocog alfa RTTE dataset. Model parameters from the adult-adolescent model and the model parameters re-estimated on the pediatric data are presented together in Table 12. Pediatric patients are characterized by a much lower baseline scale parameter A, and a shape parameter γ<1 that result in a baseline hazard decreasing over time. Pediatric patients are also characterized by a shallower drug effect. Of note, for the re-estimated RTTE model the baseline scale parameter estimate A was obtained with a higher uncertainty (>50%) than for adult/adolescent datasets, likely reflecting the lower number of patients and events in the pediatric vs adult/adolescent datasets.

TABLE 12
Model parameters of the pediatric BIVV001 RTTE model estimated
on the adult-adolescent and on the pediatric populations

| | Adult/adolescent model | Pediatric re-estimated model |
|---|---|---|
| Run | 213 | 223 |
| OFV | 1208* | 1167.75 |
| Condition number | 81.88** | 12.62 |

| | | Adult/adolescent model | | Pediatric re-estimated model | |
|---|---|---|---|---|---|
| | Unit | Value** | RSE** (%) | Value | RSE (%) |
| Constant of the base hazard (λ) | (1/h) | 0.00247 | 36.2 | 0.0000429 | 58.2 |
| Shape parameter of the base hazard (γ) | (—) | 1.02 | 6.31 | 0.694 | 14.5 |
| Constant of the FVIII effect (β) | (dL/IU) | −1.73 | 14.5 | −0.00535 | 1.55 |
| Shape parameter of the FVIII effect (α) | (—) | 0.247 | 11.2 | 1.27 | 4.37 |
| Arm effect: On-demand | (—) | 2.21 | 14.3 | — | — |
| IIV Base hazard | (CV) | 1.14 | 12.6 | 1.86 | 26.0 |

*Based on the adult/adolescent RTTE model applied (MAXEVAL = 0) to pediatric data only
**Condition number, estimates and RSEs based on adult/adolescent data
The RSE for IIV parameters are reported on the approximate SD scale.

A second covariate screening was performed on the re-estimated pediatric RTTE model to investigate if it could be further refined. The evaluated covariates included age group and bleeding history. Quality control (QC) checks were performed aiming to consolidate the reliability of the reported results. No significant covariate-parameter relationships were identified on the re-estimated RTTE model. Thus, the re-estimated RTTE model was considered the final pediatric RTTE model.

### Final Pediatric RTTE Model

The final pediatric RTTE model has the following characteristics:
Baseline hazard model: Weibull with scale parameter λ and shape parameter γ.
Drug effect model: power with parameters β, α.
Covariate model: (none).
IV model: exponential IV on λ.

The parameter estimates of the final RTTE model are shown in Table 12. The parameter estimates from the final RTTE model illustrate that Weibull base hazard with λ of 0.0000429 1/h and shape parameter γ of 0.694 is decreasing with time since γ<1. The parameter estimates also illustrate that for the power model of FVIII activity effect, the estimates of β=−0.00535 (dL/IU) and α=1.27 shows that the hazard ratio is 1.1 fold higher for FVIII activity of 1 IU/dL compared to FVIII activity of 10 IU/dL.

The following RTTE model [D2] shows the final RTTE model containing the parameter estimates for patients who are less than 12 years old:

$$h ( t ) = ( 0.0000429 * 0.694 ) * ( {( 0.0000429 * t )}^{- 0.306} ) * {e}^{- 0.00535 * {C}_{pred}^{0.00429} ( t )} \qquad [ D2 ]$$

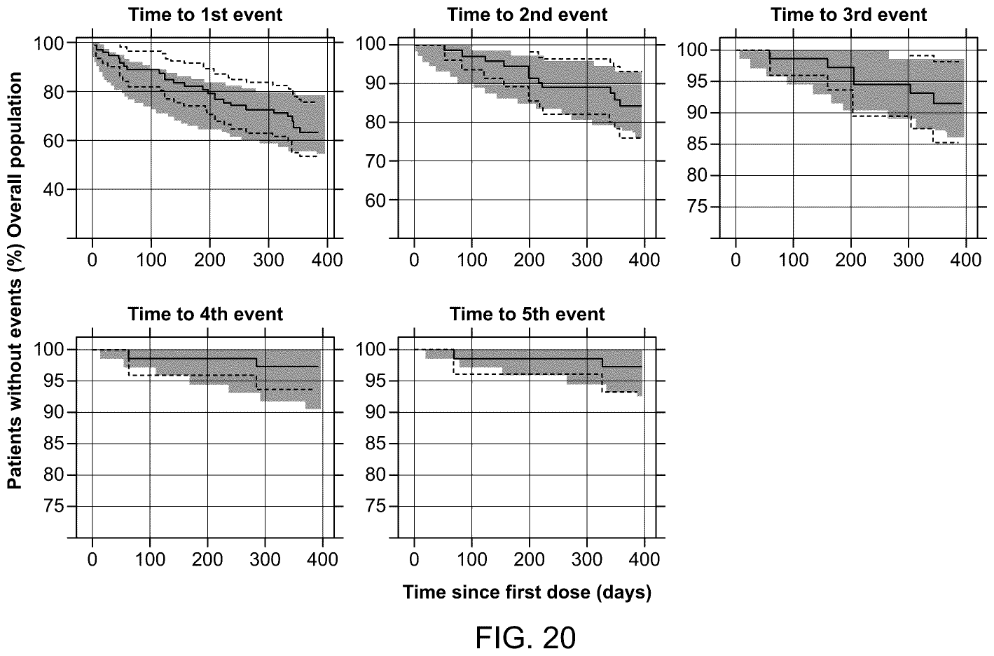

The predictive performance of the adult/adolescent RTTE model was also assessed in pediatric hemophilia A patients using visual predictive check (VPC), i.e., by keeping the model parameters fixed to the estimates in the adult/adolescent population. The VPC for the final RTTE model showed that this model adequately described the data for the full pediatric data set (see FIG. 20). The VPC for the final RTTE model also adequately described the data for both age groups (data not shown).

### Final Pediatric RTTE Model Testing:

Probability of first bleed in 1 year simulation: The final pediatric BIVV001 RTTE model was used to predict the probability of first bleed in one year for a range of BIVV001 prophylactic dosing regimens in typical pediatric patients from both age groups. The body weight for the typical patients <6 years and 6 to <12 years were 18 kg and 32.8 kg, respectively, which correspond to the median observed values in study EFC16295. Uncertainty in the final RTTE model parameters was included in these simulations using 300 SIR samples of RTTE parameter sets. The results for selected dosing regimens are shown in Tables 13 and 14. Continuous infusion is considered dose infused over 1 week, e.g. for 10 IU/kg continuous infusion, for an 18 kg patient, 180 IU were infused over 1 week and continued for 52 weeks; for a 32.8 kg patient, 328 IU were infused over 1 week and continued for 52 weeks.

TABLE 13
Probability of first bleed in 1 year for selected
BIVV001 prophylaxis dosing regimens in a typical
pediatric patient <6 years weighing 18 kg

| | | | | Probability |
|---|---|---|---|---|
| | | Fraction | Fraction | (%) of |
| | FVIII | (%) of | (%) of | first bleed |
| | Activity | time when | time when | (median [95% |
| | (IU/dL) | FVIII >10 | FVIII >40 | CI]) at |
| Regimen | C_{trough} | IU/dL | IU/dL | 52 weeks |
| 25 IU/kg, q4 days | 11.700 | 99.9 | 23.6 | 29 [14, 45] |
| 15 IU/kg continuous | 10.000 | 96.3 | 0.0 | 38 [20, 53] |
| 50 IU/kg, q7 days | 5.870 | 81.0 | 32.4 | 28 [14, 44] |
| 30 IU/kg, q7 days | 3.520 | 63.1 | 14.5 | 33 [17, 49] |
| 50 IU/kg, q14 days | 0.322 | 39.5 | 15.2 | 34 [18, 51] |
| 10 IU/kg continuous | 6.690 | 0.0 | 0.0 | 39 [20, 55] |
| 12 IU/kg continuous | 8.030 | 0.0 | 0.0 | 38 [20, 54] |

As shown in Table 13, the results predicted that for a typical pediatric patient <6 years and weighing 18 kg, receiving 50 IU/kg QW for 1 year prophylactically, the probability of having a first bleed in 1 year (95% CI) was 28% (14%-44%), with a steady state trough concentration C_{trough}. Time >10 IU/dL (% of dosing interval) and Time >40 IU/dL (% of dosing interval) of 5.87 IU/dL, 81% and 32% respectively. The probability of first bleed in 1 year illustrated that the high sustained FVIII activity that is achieved by 50 IU/kg QW regimen, lowered the probability of first bleed in 1 year from 38% to 28% compared to the 10-15 IU/kg continuous infusion regimens in the typical pediatric patient <6 years weighing 18 kg.

TABLE 14
Probability of first bleed in 1 year for selected
BIVV001 prophylaxis dosing regimens in a typical
pediatric patient 6 to <12 years weighing 32.8 kg

| | | | | Probability |
|---|---|---|---|---|
| | | Fraction | Fraction | (%) of |
| | FVIII | (%) of | (%) of | first bleed |
| | Activity | time when | time when | (median [95% |
| | (IU/dL) | FVIII >10 | FVIII >40 | CI]) at |
| Regimen | C_{trough} | IU/dL | IU/dL | 52 weeks |
| 25 IU/kg, q4 days | 15.200 | 100.0 | 35.7 | 27 [13, 42] |
| 15 IU/kg continuous | 12.200 | 98.8 | 0.0 | 37 [19, 52] |
| 50 IU/kg, q7 days | 8.140 | 92.1 | 39.9 | 26 [13, 41] |
| 30 IU/kg, q7 days | 4.890 | 72.8 | 20.7 | 31 [16, 48] |
| 50 IU/kg, q14 days | 0.533 | 44.8 | 18.7 | 33 [17, 49] |
| 10 IU/kg continuous | 8.130 | 0.0 | 0.0 | 38 [20, 54] |
| 12 IU/kg continuous | 9.750 | 0.0 | 0.0 | 38 [20, 54] |

As shown in Table 14, the results predicted that for a typical pediatric patient 6 to <12 years and weighing 32.8 kg, receiving 50 IU/kg QW for 1 year prophylactically, the probability of having a first bleed in 1 year (95% CI) was 26% (13%-41%), with a steady state C_{trough}. Time >10 IU/dL (% of dosing interval) and Time >40 IU/dL (% of dosing interval) of 8.14 IU/dL, 92% and 40% respectively. The probability of first bleed in 1 year illustrated that the high sustained FVIII activity that is achieved by 50 IU/kg QW regimen, lowered the probability of first bleed in 1 year from 37% to 26% compared to the 10-15 IU/kg continuous infusion regimens in the typical pediatric patient 6 to <12 years weighing 32.8 kg.

The results of this simulation demonstrate the advantage of achieving normal to near-normal FVIII activities (>40 IU/dL) after the 50 IU/kg QW regimen, which was covered by 32% and 40% of time in the pediatric patients <6 years and 6 to <12 years of age, respectively, as compared to 0% of time after 10-15 IU/kg continuous infusion in both age groups.

Annualized bleed rate (ABR): Simulations were conducted with the final pediatric RTTE model in a virtual population of 10000 pediatric patients <6 years old and 10000 pediatric patients 6 to <12 years old treated with 50 IU/kg QW prophylaxis regimen of BIVV001 up to 1 year (52 weeks). The individual simulated ABR was computed from simulations. The virtual populations <6 years and 6 to <12 years had a mean (SD) bodyweight of 16.2 kg (4.73 kg) and of 35.5 kg (13.6 kg), respectively. The descriptive statistics of the simulated ABR are shown in Table 15.

The simulated mean ABR in the overall pediatric population was 0.74 and median ABR was 0 (Q1-Q3: 0-1). This is in good agreement with the observed ABR for the study EFC16295 in which the mean (95% CI) ABR was 0.89 (0.56, 1.42) and median ABR was 0. In the age groups <6 years and 6 to <12 years the simulated mean ABRs were 0.76 and 0.70, respectively, which were within the 95% CI around the observed mean ABRs of 0.48 (0.30; 0.77) and 1.33 (0.64; 2.76), respectively. In the simulations, 63.6% and 65.6% of the virtual patients <6 years and 6 to <12 years, respectively, had 0 bleeds which is similar to the percentage in study EFC16295 where 63.2% (24 out of 38) and 63.8% (23 out of 36) patients were observed to have 0 bleeds during the efficacy period in the same age groups.

TABLE 15
Descriptive statistics of the simulated ABR (for 52 weeks) in virtual
population receiving 50 IU/kg QW BIVV001 as prophylaxis, by age group

| ABR at one year | Min | 2.5% | 25% | Median | 75% | 97.5% | Max | Mean | SD |
|---|---|---|---|---|---|---|---|---|---|
| Age < 6 years | 0 | 0 | 0 | 0 | 1 | 5 | 46 | 0.76 | 1.79 |
| Age 6 to <12 years | 0 | 0 | 0 | 0 | 1 | 4 | 52 | 0.70 | 1.75 |
| All | 0 | 0 | 0 | 0 | 1 | 5 | 52 | 0.74 | 1.78 |

A sensitivity analysis was performed excluding the participant with an outlier number of treated bleeds in the 6 to <12 years of age cohort. The mean ABR estimated from the negative binomial model decreased to 0.75 (95% CI: 0.41 to 1.40) in the 6 to <12 years of age cohort, and to 0.61 (95% CI: 0.42 to 0.90) overall.

These simulations show that the performance of the final pediatric RTTE model was acceptable, and it can be used to predict the bleeding profile and ABR in the first year for a pediatric population (<12 years) who are on 50 IU/kg QW BIVV001 prophylactically.

### Conclusions:

The bleeding events in pediatric hemophilia A patients treated with BIVV001 were adequately characterized by the final pediatric RTTE model [Model D2] with a Weibull baseline hazard from which the effect of OSC FVIII activity on baseline hazard was modeled with a power relationship. Diagnostic plots showed that the percentage of pediatric patients experiencing bleeding events with time was generally well described by the adult/adolescent RTTE model, but the time to first bleed was slightly underpredicted in younger patients (<6 years), i.e., the bleed events in this age group were predicted to happen slightly earlier than observed. A covariate search performed on the RTTE model with fixed parameters to the adult/adolescent estimates using the SCM procedure did not identify significant covariate-parameter relationships. The fit of the pediatric data to the adult/adolescent RTTE model was improved by re-estimating the model parameters on the pediatric efanesoctocog alfa RTTE dataset. An additional covariate search on the re-estimated RTTE model did not lead to further model refinement, therefore Model [D2] was considered as the final pediatric RTTE model.

The final pediatric RTTE model performed well capturing the time to bleed (1st to 5th events) in the pediatric population. Although the observed time to first bleed was still at the upper edge of the prediction interval for the younger patients (<6 years), the model was better suited to describe the ABRs in this age group with a simulated mean ABR of 0.76 vs 1.32 based on the adult/adolescent RTTE model compared to the observed mean (95% CI) ABR=0.48 (0.30; 0.77).

Thus, the bleeding events in pediatric hemophilia A patients treated with efanesoctocog alfa were adequately characterized by an RTTE model with a Weibull baseline hazard from which the effect of OSC FVIII activity on baseline hazard was modeled with a power relationship.

### Example 4. Population Pharmacokinetic (popPK) Model Application to Pediatric Patients

The objective of the following analysis was to use empirical Bayes estimates (EBEs) of the PK parameters based on the popPK model (Model [A]) described in Example 2 to describe the FVIII activity from the EFC16295 study. This study included only pediatric patients less than 12 years of age. Descriptive statistics of potential covariates (baseline values) of the patients from the EFC16295 study included in the MAP Bayesian analysis is summarized in Table 16 and Table 17.

TABLE 17
Descriptive statistics of the continuous covariates
in EFC16295 in the Final Dataset

| Covariate | Descriptive | EFC16295 | |
|---|---|---|---|
| candidate | statistic | (N = 74) | |
| Baseline | Mean (SD) | 26.6 | (12.9) |
| bodyweight (kg) | Median (5th-95th percentile) | 22.0 | (13.2-55.6) |
| Baseline age (year) | Mean (SD) | 5.99 | (2.91) |
| | Median (5th-95th percentile) | 5.00 | (2.00-11.0) |
| Baseline | Mean (SD) | 36.0 | (2.59) |
| hematocrit (%) | Median (5th-95th percentile) | 36.0 | (32.0-40.3) |
| Baseline VWF | Mean (SD) | 80.3 | (27.8) |
| | Median (5th-95th percentile) | 75.0 | (48.3-131) |

TABLE 17
Descriptive statistics of the categorical covariates for
the subjects in EFC16295 study in the Final Dataset

| Covariate | N | Type | n (%) | |
|---|---|---|---|---|
| Age group | 74 | Older pediatric (6 years ≤ | 36 | (48.7) |
| | | Age < 12 years) | | |
| | | Younger pediatric (Age < 6 years) | 38 | (51.3) |
| Hepatitis C | 74 | With | 0 | (0) |
| infection status | | Without | 74 | (100) |
| HIV infection | 74 | With | 0 | (0) |
| status | | Without | 74 | (100) |
| Race | 74 | Caucasian | 55 | (74.3) |
| | | Asian | 8 | (10.8) |
| | | Black | 3 | (4.05) |
| | | Other race | 4 | (5.41) |
| | | Race not reported | 4 | (5.41) |
| Blood type | 74 | Blood type A | 21 | (28.4) |
| | | Blood type AB | 2 | (2.7) |
| | | Blood type B | 8 | (10.8) |
| | | Blood type O | 26 | (35.1) |
| | | Blood type unknown | 17 | (23.0) |

Baseline bodyweight in pediatric patients (1.4 years to <12 years) from EFC16295 study ranged from 11.4 kg to 66.5 kg. There were 2, 36, and 36 patients in the age range of <2 years, 2 years sage <6 years, and 6 years sage <12 years, respectively.

Baseline VWF for pediatric patients (from EFC16295) was lower than those for adult and adolescent patients from all other studies.

In EFC16295, there were approximately 74% Caucasians, 11% Asians, 4% Blacks, 5% Other and 5% of the patients did not report their Race. For the distribution of blood type, ˜28% of patients had blood type A, ˜35% had blood type O, ˜11% had blood type B, ˜3% had blood type AB and rest ˜23% had unknown blood type. No patients in EFC16295 were HCV positive or HIV positive.

### Application of PopPK Model Described in Example 2 to EFC16295

A popPK model (Model [A]) was used to describe the FVIII activity data from EFC16295 using MAXEVAL=0 option in NONMEM. The parameterization in the popPK model is:

$$CL = 0.433 {( \frac{Bodyweight ( kg )}{78.3} )}^{0.677} {( 0.896 )}^{Asian} {e}^{{η}_{1}} \qquad [ {A}^{′} ]$$

$$V = 30.2 {( \frac{Bodyweight ( kg )}{78.3} )}^{0.791} {e}^{{η}_{2}}$$

Where Asian=1 for Asians and Asian=0 for non-Asians.

The model performance was assessed based on the goodness of fit (GOF), visual predictive checks (VPCs), and measures of quality criteria.

PopPK models [A] and [A′] are useful for subjects of all ages.

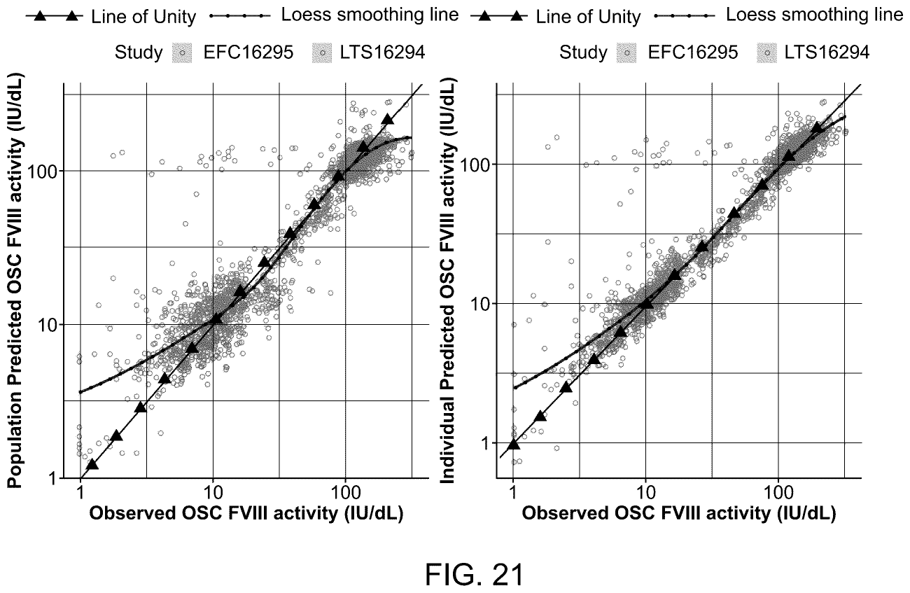

As shown in FIG. 21, the population predicted OSC FVIII activity (PRED) vs observed (DV) data suggests the appropriateness of the previous popPK model to describe the FVIII activity vs time from EFC16295 and LTS16294 studies. The individual predicted OSC FVIII activity (IPRED) vs observed (DV) plot shows that the interindividual variability model was able to explain the variability in the predictions.

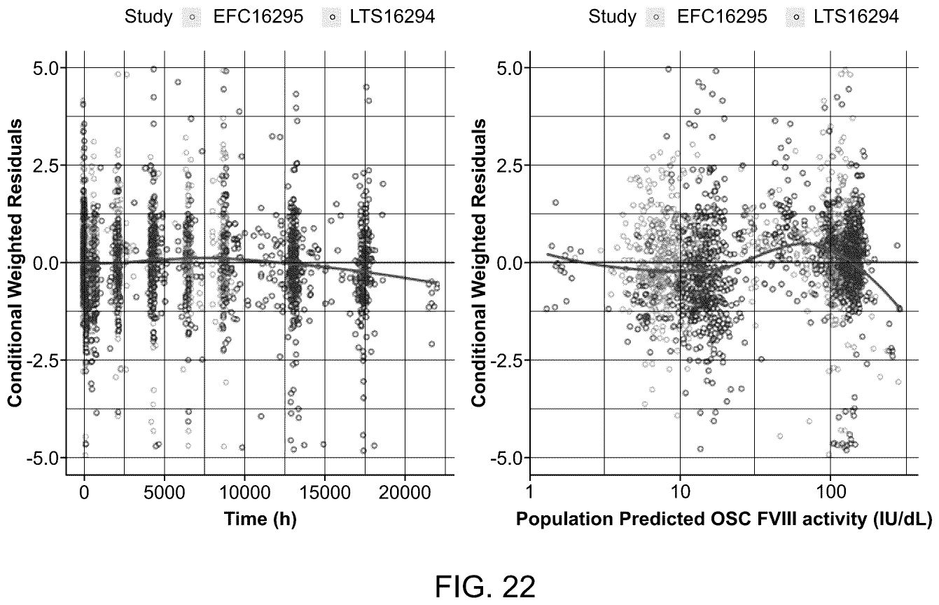

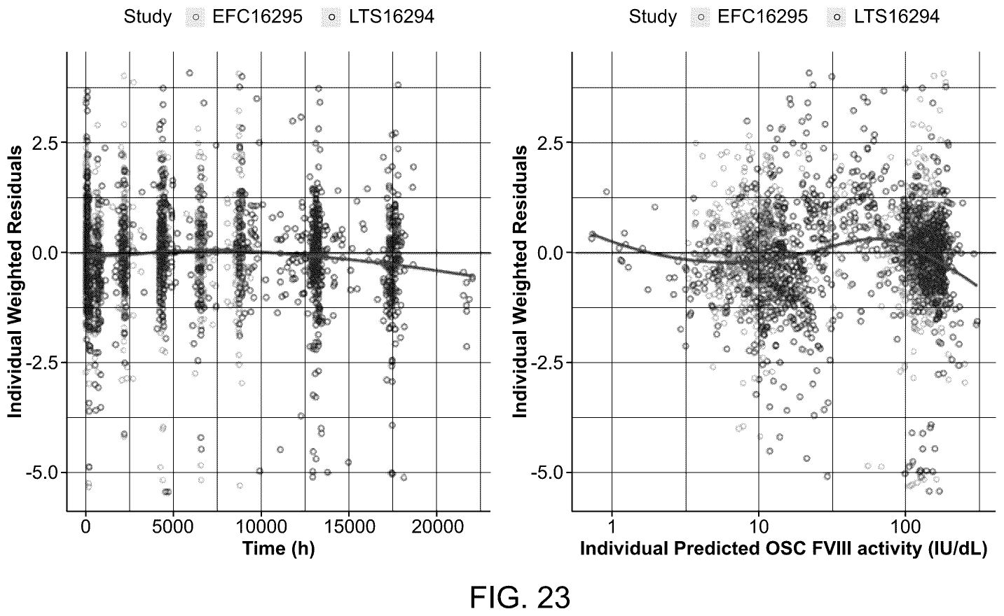

FIG. 22 and FIG. 23 show the residuals variation with time and predicted FVIII activity. Both the CWRES and IWRES are well/evenly distributed around 0, when plotted with Time for the entire duration or when plotted with PRED and IPRED respectively.

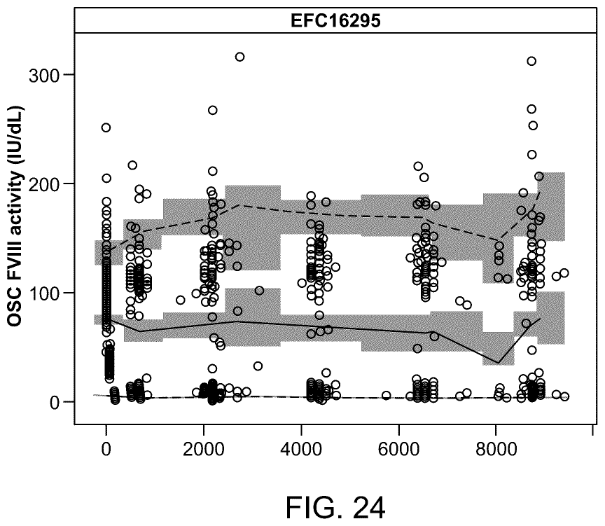

VPC was used evaluate the performance of the final popPK model with the EFC16295 study data. VPC graphs are presented in FIG. 24. The results of the VPC showed that a majority of the observed FVIII activities were within in the prediction range [5th-95th percentiles] and only a few FVIII activity data points were outside of the predicted percentile range [5th-95^{th} percentiles].

In addition, Table 18 shows the quality criteria summarized for EFC16295 study. Overall, the MPE as a measure of bias was <10% for PRED and <2% for IPRED, whereas the RMSE was <36.2% for PRED and <28.4% for IPRED. Both MPE and RMSE were considered acceptable for model performance in describing the FVIII activity for EFC16295.

TABLE 18
Summary of mean prediction error (MPE) and root mean square error (RMSE)

| | | | | MPE 95% CI | | | RMSE 95% CI |
|---|---|---|---|---|---|---|---|
| | | MPE | MPE | (lower bound; | RMSE | RMSE | (lower bound; |
| | STUDY | (absolute) | (%) | upper bound) | (absolute) | (%) | upper bound) |
| PRED | EFC16295 | −4.35 | 6.05 | −6.03; −2.66 | 26.0 | 36.2 | 22.3; 29.3 |
| IPRED | EFC16295 | −0.177 | −0.25 | −1.52; 1.16 | 20.4 | 28.4 | 17.3; 23.2 |

### Final PopPK Model Performance for Peri-Operative FVIII Activity

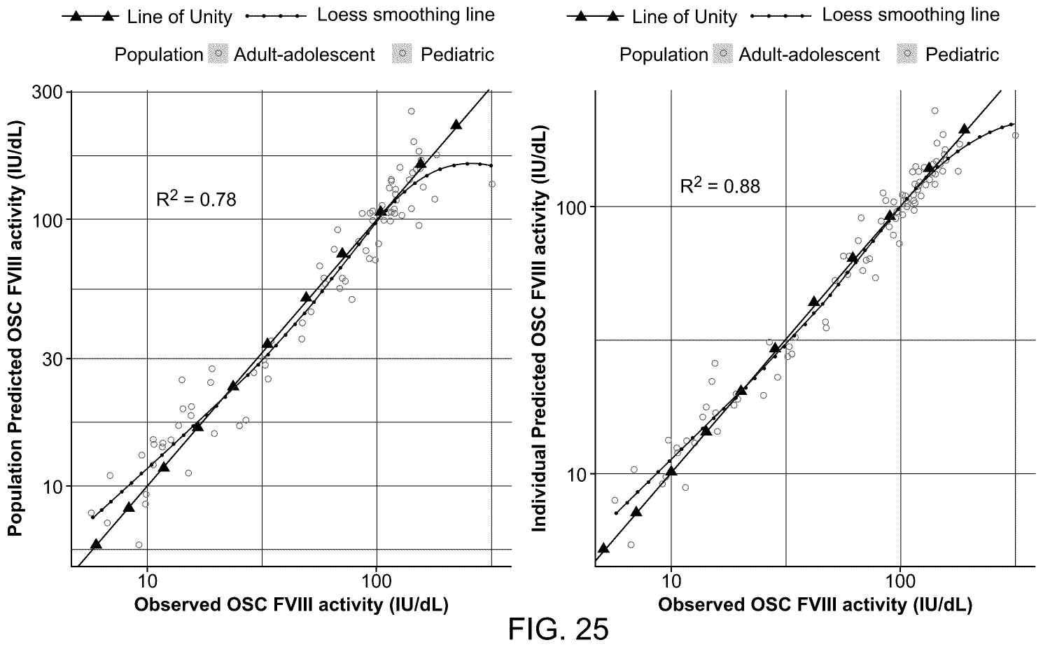

There were 32 patients (from EFC16295 and LTS16294) who underwent surgeries during the efanesoctocog alfa treatment. As per protocol, these patients received ad hoc efanesoctocog alfa doses during the pre-operative treatment and post-operative follow-up and PK samples were collected to assess the patient's FVIII activity during and after the surgical procedure. FIG. 25 shows the population predicted (PRED) vs observed and individual predicted (IPRED) vs observed FVIII activity plots respectively for FVIII activity assessed during peri-operative period. As seen from FIG. 25, both the PRED and IPRED were able to reasonably describe the FVIII activity data, with a R2 value of 0.78 and 0.88 respectively. The adult-adolescent patients with surgery in FIG. 25 are from LTS16294 study.

### EFC16295 Post-Hoc Intrinsic or Extrinsic Factor Assessment

Once the final popPK model was validated using GOF, VPC and the referenced quality criteria, exploration was conducted using post-hoc exposure parameters with selected intrinsic and extrinsic factors for the EFC16295 study. Steady state AUCₜₐᵤ (AUC0-168 h), Cₘₐₓ and Cₘᵢₙ were used as FVIII exposure parameters generated for week 26 for 73 patients in EFC16295. One patient did not continue until week 26 so that patient's exposure at steady state could not be computed.

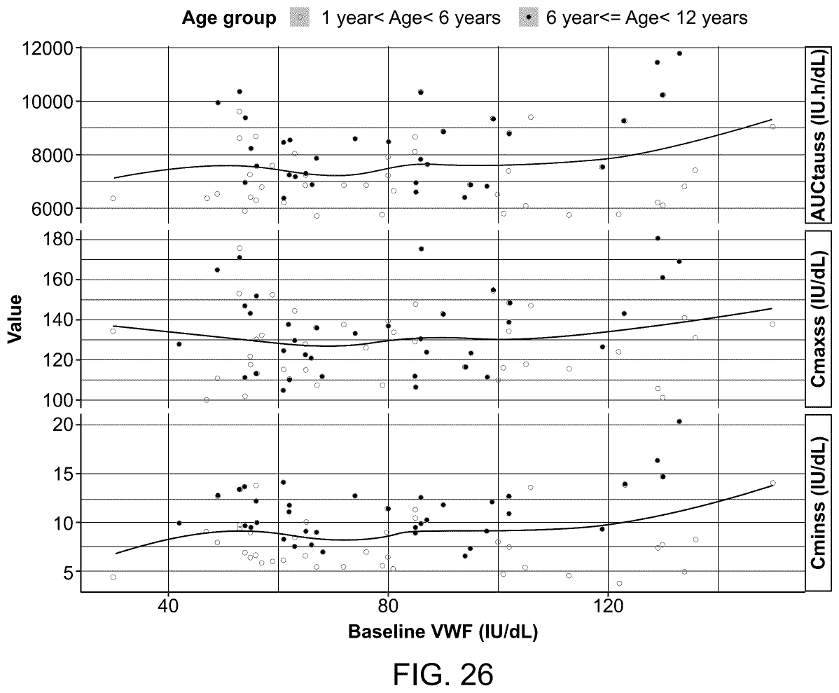

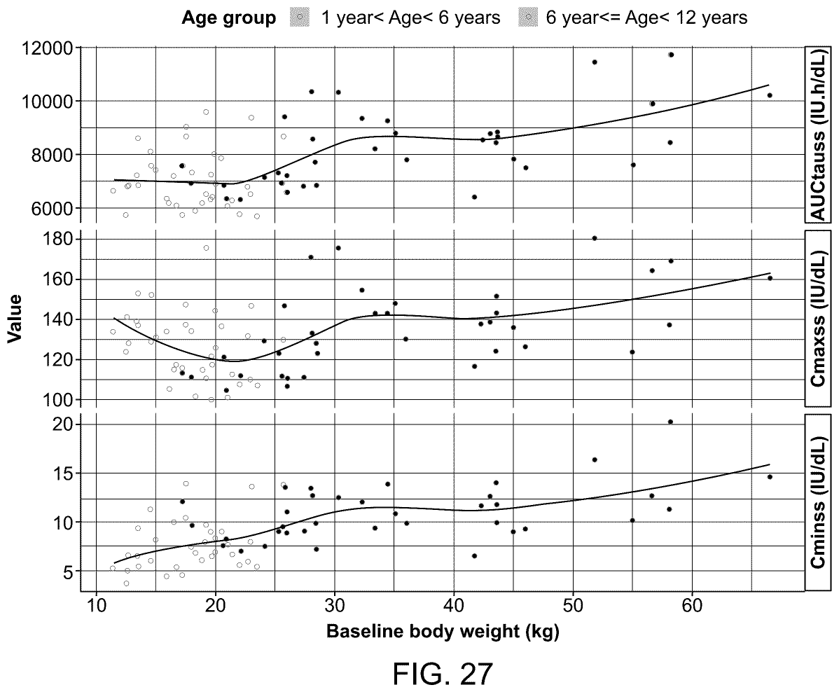

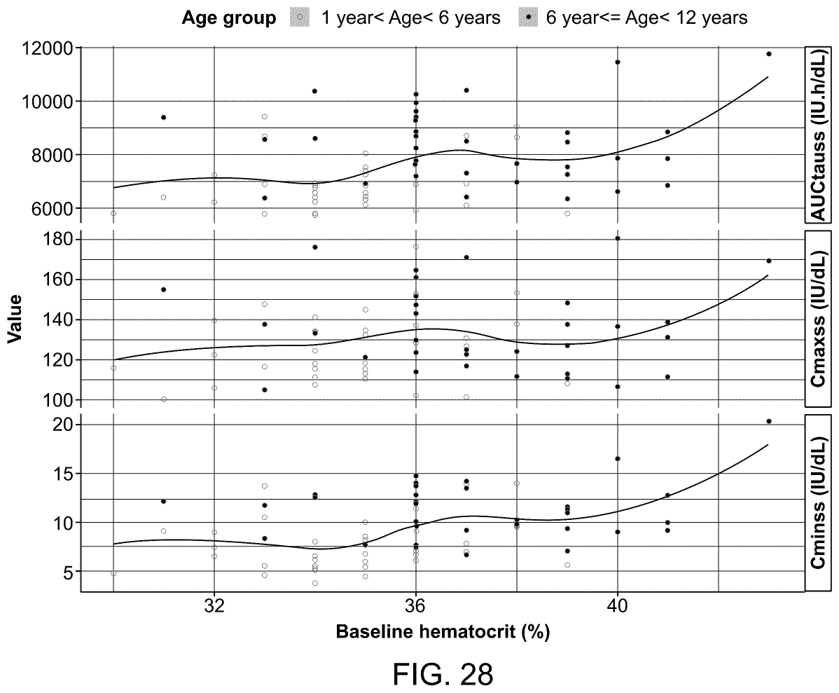

When the steady state AUC (AUCₜₐᵤₛₛ), Cₘₐₓ (Cₘₐₓₛₛ) and Cₘᵢₙ (Cₘᵢₙₛₛ) were plotted with baseline VWF (BVWF) and baseline hematocrit (BH), no specific trends for both younger and older children were observed with either BVWF or BH, as shown in FIGS. 26-28. As expected, a trend of increasing AUCₜₐᵤₛₛ, Cₘₐₓₛₛ and Cₘᵢₙₛₛ were observed with increasing bodyweight due to the bodyweight effect already incorporated in the popPK model on CL and V parameters. This is consistent with the predicted trend of increasing FVIII exposure with increasing bodyweight.

Similarly, the steady state FVIII exposure parameters were evaluated with categorical factors such as blood type A and blood type O. No major trends were observed for the steady state exposure with blood type A or blood type O.

The other categorical factors were not used for assessment due to less diversity of the patients with those categorical factors. For example, in the EFC16295 study, there were no patients with HCV infection or HIV infection. Similarly, for blood type B, 8 out of 73 patients were with blood type B, while for only 2 out of 73 patients had blood type AB.

### Post-Hoc PK Parameters

Using post-hoc PK parameters, exposure parameters such as Cₘₐₓ and Cₘᵢₙ (also called C_{trough}) were derived by simulating each individual's FVIII activity profile with time for the actual doses that were administered. The simulation was conducted to derive day 1 as well as steady state Cₘₐₓ and Cₘᵢₙ for patients in EFC16295. FVIII activity was simulated until the week 26 dose to obtain steady state Cₘₐₓ and Cₘᵢₙ. Out of 74 patients in EFC16295, 73 patients had dosing beyond week 26 and were included in steady state calculations.

Table 19, shows the comparison of summary statistics for Cₘₐₓ and Cₘᵢₙ after day 1 dose, stratified by age group. The Cₘₐₓ and Cₘᵢₙ predicted from the PopPK analysis were comparable to NCA derived or observed Cₘₐₓ and Cₘᵢₙ in EFC16295, across both age groups.

Mean (SD) values for PK parameters derived from population PK post-hoc assessment and non-compartmental analysis (NCA) are shown in Table 20, which shows that CL and V increase, and t½ decreases with decreasing age for PK parameters derived both from population PK and NCA. Overall, the mean and SD for the t½ was generally comparable between population PK and NCA for each age group, while CL and V were lower from the NCA estimation compared to PopPK for each age group.

These results illustrate that popPK model predicts the FVIII profile with time, individual PK and exposure parameters and across-patient variability reasonably well.

TABLE 19
Comparison on PopPK and Non-compartment analysis (NCA)
derived PK Parameters for baseline-corrected FVIII activity
after day 1 dose in PK group in EFC16295 patients

| | | PopPK post-hoc PK | | NCA or observed PK | |
|---|---|---|---|---|---|
| | | parameters day 1 | | parameters day 1 | |
| | | Cₘₐₓ | Cₘᵢₙ | Cₘₐₓ | Cₘᵢₙ |
| | | (IU/dL) | (IU/dL) | (IU/dL)* | (IU/dL) ‡ |
| 6 years ≤ | N | 18 | 18 | 18 | 17 |
| Age < 12 | Mean | 107 | 8.43 | 113 | 6.93 |
| years | SD | 14.1 | 1.83 | 22.7 | 1.76 |
| Age < 6 | N | 19 | 19 | 19 | 16 |
| years | Mean | 108 | 6.43 | 143 | 5.73 |
| | SD | 19.1 | 1.82 | 57.8 | 1.63 |

†Predicted Cmin is the post-hoc simulated FVIII activity at time = 168 h after day 1 dose.
Predicted Cmax is the post-hoc simulated Cmax after day 1 dose.
‡ Observed Cmin is the baseline visit day 8 FVIII activity
*Observed Cmax is obtained from NCA analysis.

TABLE 20
Comparison of PK parameters derived from population
PK analysis and non-compartmental analysis

| | Mean (SD) post-hoc PK | | | | Mean (SD) PK | | | |
|---|---|---|---|---|---|---|---|---|
| | parameters from PopPK | | | | parameters from NCA | | | |
| Age | | CL | V | t½ | | CL | Vss | t½z |
| group | N | (mL/h/kg) | (mL/kg) | (h) | N | (mL/h/kg) | (mL/kg) | (h) |
| <6 years | 19 | 0.813 | 48.2 | 41.3 | 18 | 0.742 | 36.6 | 38.0 |
| | | (0.110) | (6.65) | (4.11) | | (0.121) | (5.59) | (3.72) |
| 6 years ≤ | 18 | 0.723 | 47.6 | 45.7 | 18 | 0.681 | 38.1 | 42.4 |
| Age < 12 | | (0.0916) | (5.11) | (2.64) | | (0.139) | (6.80) | (3.70) |
| years | | | | | | | | |

Abbreviations: CL = clearance, t½ = half-life, NCA = noncompartmental analysis; PK = pharmacokinetic(s); t½z = terminal half-life, V = volume of distribution, Vss = volume of distribution at steady state.

### Time to FVIII Activity

The steady state Time to FVIII activity increases as FVIII activity threshold decreases from 150 IU/dL to 1 IU/dL. This was observed in both pediatric age groups, as shown in Table 21.

TABLE 21
Descriptive statistics of steady state (week 26) TIME to pre-defined FVIII activity for EFC16295

| | | | | | | | |
|---|---|---|---|---|---|---|---|
| Time to 1 | Time to 3 | Time to 5 | Time to 10 | Time to 15 | Time to 20 | Time to 40 | Time to 150 |
| IU/dL FVIII | IU/dL FVIII | IU/dL FVIII | IU/dL FVIII | IU/dL FVIII | IU/dL FVIII | IU/dL FVIII | IU/dL FVIII |
| activity (h) | activity (h) | activity (h) | activity (h) | activity (h) | activity (h) | activity (h) | activity (h) |

Age < 6 years

| | | | | | | | | |
|---|---|---|---|---|---|---|---|---|
| n | 37 | 37 | 37 | 37 | 37 | 37 | 37 | 3 |
| Mean | 287 | 222 | 192 | 150 | 126 | 109 | 68.0 | 3.80 |
| SD | 33.2 | 25.9 | 22.6 | 18.2 | 15.7 | 14.0 | 10.5 | 4.66 |
| Geo. Mean | 285 | 220 | 190 | 149 | 125 | 108 | 67.3 | 2.20 |
| % CV | 11.5 | 11.7 | 11.8 | 12.1 | 12.5 | 12.9 | 15.4 | 122 |
| Median | 284 | 219 | 188 | 147 | 123 | 106 | 64.8 | 1.42 |
| Minimum | 231 | 178 | 154 | 121 | 101 | 87.5 | 54.3 | 0.82 |
| Maximum | 364 | 282 | 244 | 192 | 162 | 141 | 91.8 | 9.17 |

6 years ≤ Age < 12 years

| | | | | | | | | |
|---|---|---|---|---|---|---|---|---|
| n | 36 | 36 | 36 | 36 | 36 | 36 | 36 | 8 |
| Mean | 327 | 253 | 219 | 173 | 146 | 127 | 80.6 | 6.81 |
| SD | 27.0 | 22.2 | 20.0 | 17.1 | 15.6 | 14.5 | 12.3 | 4.22 |
| Geo. Mean | 325 | 252 | 218 | 172 | 145 | 126 | 79.7 | 4.90 |
| % CV | 8.28 | 8.75 | 9.10 | 9.90 | 10.7 | 11.5 | 15.3 | 62.0 |
| Median | 324 | 251 | 217 | 172 | 144 | 125 | 78.4 | 7.31 |
| Minimum | 277 | 213 | 184 | 143 | 120 | 103 | 62.3 | 0.55 |
| Maximum | 406 | 319 | 279 | 224 | 192 | 169 | 114 | 13.0 |

Abbreviations: SD is standard deviation, % CV is percent co-efficient of variation

For pediatric patients with 6 years ≤Age <12 years, the median Time to FVIII activity is 324 h (13.5 days), 172 h (7.17 days) and 78.4 h (3.27 days) for the FVIII activity thresholds of 1 IU/dL, 10 IU/dL and 40 IU/dL respectively.

For pediatric patients with Age <6 years, the median Time to FVIII activity is 284 h (11.8 days), 147 h (6.13 days) and 64.8 h (2.70 days) for the FVIII activity thresholds of 1 IU/dL, 10 IU/dL and 40 IU/dL respectively.

In the post-hoc simulations, 8 patients with 6 years ≤Age <12 years age and 3 patients with Age <6 years were predicted to have peak FVIII activity >150 IU/dL at steady state.

The descriptive statistics of day 1 Time to FVIII activity for patients in PK group for both pediatric age groups are shown in Table 22.

TABLE 22
Descriptive statistics of day 1 Time to pre-defined FVIII activity for patients in PK group in EFC16295

| | | | | | | | |
|---|---|---|---|---|---|---|---|
| Time to 1 | Time to 3 | Time to 5 | Time to 10 | Time to 15 | Time to 20 | Time to 40 | Time to 150 |
| IU/dL FVIII | IU/dL FVIII | IU/dL FVIII | IU/dL FVIII | IU/dL FVIII | IU/dL FVIII | IU/dL FVIII | IU/dL FVIII |
| activity (h) | activity (h) | activity (h) | activity (h) | activity (h) | activity (h) | activity (h) | activity (h) |

Age < 6

| | | | | | | | | |
|---|---|---|---|---|---|---|---|---|
| n | 19 | 19 | 19 | 19 | 19 | 19 | 19 | 1 |
| Mean | 278 | 213 | 182 | 141 | 117 | 99.6 | 58.3 | 7.8 |
| SD | 26.2 | 20.1 | 17.5 | 14 | 12.2 | 11.1 | 9.11 | NC |
| Geo. Mean | 277 | 212 | 181 | 140 | 116 | 99 | 57.7 | 7.8 |
| % CV | 9.41 | 9.48 | 9.59 | 9.97 | 10.5 | 11.1 | 15.6 | NC |
| Median | 281 | 214 | 182 | 139 | 115 | 98.7 | 56.9 | 7.8 |
| Minimum | 240 | 185 | 159 | 123 | 102 | 86.2 | 48.6 | 7.8 |
| Maximum | 350 | 268 | 230 | 179 | 149 | 127 | 84 | 7.8 |

6 years ≤ Age < 12 years

| | | | | | | | | |
|---|---|---|---|---|---|---|---|---|
| n | 18 | 18 | 18 | 18 | 18 | 18 | 18 | 0 |
| Mean | 308 | 235 | 202 | 156 | 129 | 110 | 64.4 | NC |
| SD | 20.9 | 17.2 | 15.6 | 13.6 | 12.5 | 11.7 | 10.3 | NC |
| Geo. Mean | 307 | 235 | 201 | 155 | 129 | 110 | 63.5 | NC |
| % CV | 6.78 | 7.32 | 7.75 | 8.7 | 9.65 | 10.7 | 16 | NC |
| Median | 308 | 234 | 200 | 154 | 127 | 108 | 63.5 | NC |
| Minimum | 270 | 205 | 175 | 134 | 106 | 86.6 | 39.6 | NC |
| Maximum | 346 | 270 | 234 | 186 | 158 | 138 | 89.5 | NC |

ALL

| | | | | | | | | |
|---|---|---|---|---|---|---|---|---|
| n | 37 | 37 | 37 | 37 | 37 | 37 | 37 | 1 |
| Mean | 293 | 224 | 192 | 148 | 123 | 105 | 61.2 | 7.8 |
| SD | 27.9 | 21.8 | 19.1 | 15.6 | 13.7 | 12.5 | 10.1 | NC |
| Geo. Mean | 291 | 223 | 191 | 147 | 122 | 104 | 60.5 | 7.8 |
| % CV | 9.52 | 9.75 | 9.97 | 10.5 | 11.2 | 11.9 | 16.4 | NC |
| Median | 292 | 222 | 190 | 147 | 122 | 103 | 59.6 | 7.8 |
| Minimum | 240 | 185 | 159 | 123 | 102 | 86.2 | 39.6 | 7.8 |
| Maximum | 350 | 270 | 234 | 186 | 158 | 138 | 89.5 | 7.8 |

Conclusions: Based on the GOF, VPC and quality criteria, the popPK model was considered as appropriate to characterize the FVIII activity data from pediatric study. In addition, the model was also able to describe the FVIII activity data collected during ad-hoc dosing during peri-operative management in pediatric and long-term safety studies. The influence of selected intrinsic and extrinsic factors on FVIII activity in pediatric patients was evaluated by the comparison of post hoc estimates of FVIII activity exposures.

Consistent with the previous finding, the pediatric post-hoc steady state FVIII activity exposures varied significantly only with bodyweight, as bodyweight effect was included on CL and V in the popPK model. All other evaluated factors, such as baseline VWF, baseline hematocrit, blood types A and O had no apparent effect on pediatric FVIII activity based on available data. FVIII activity in pediatric and long-term safety studies was well described by the previous popPK model, with the effect of body weight on exposure as the only source of apparent FVIII variability across the pediatric population.

Using MAP Bayesian approach, the additional FVIII activity data for pediatric patients in the EFC16295 study was adequately characterized by the popPK model developed previously for adult/adolescent patients, as described in Example 2.

TABLE A
Efanesoctocog alfa Sequence Information

| Sequence | |
|---|---|
| Description / | |
| SEQ ID NO. | Amino acid or nucleic acid sequence |
| rFVIIIFc-ELNN | MQIELSTCFF LCLLRFCFSA TRRYYLGAVE LSWDYMQSDL GELPVDARFP PRVPKSFPEN 60 |
| amino acid | TSVVYKKTLF VEFTDHLFNI AKPRPPWMGL LGPTIQAEVY DTVVITLKNM ASHPVSLHAV 120 |
| sequence, | GVSYWKASEG AEYDDQTSQR EKEDDKVFPG GSHTYVWQVL KENGPMASDP LCLTYSYLSH 180 |
| including signal | VDLVKDLNSG LIGALLVCRE GSLAKEKTQT LHKFILLFAV FDEGKSWHSE TKNSLMQDRD 240 |
| peptide | AASARAWPKM HTVNGYVNRS LPGLIGCHRK SVYWHVIGMG TTPEVHSIFL EGHTFLVRNH 300 |
| (SEQ ID NO: 1) | RQASLEISPI TFLTAQTLLM DLGQFLLFCH ISSHQHDGME AYVKVDSCPE EPQLRMKNNE 360 |
| | EAEDYDDDLT DSEMDVVRED DDNSPSFIQI RSVAKKHPKT WVHYIAAEEE DWDYAPLVLA 420 |
| | PDDRSYKSQY LNNGPQRIGR KYKKVRFMAY TDETFKTREA IQHESGILGP LLYGEVGDTL 480 |
| | LIIFKNQASR PYNIYPHGIT DVRPLYSRRL PKGVKHLKDF PILPGEIFKY KWTVTVEDGP 540 |
| | TKSDPRCLTR YYSSFVNMER DLASGLIGPL LICYKESVDQ RGNQIMSDKR NVILFSVEDE 600 |
| | NRSWYLTENI QRFLPNPAGV QLEDPEFQAS NIMHSINGYV FDSLQLSVCL HEVAYWYILS 660 |
| | IGAQTDFLSV FFSGYTFKHK MVYEDTLTLF PFSGETVFMS MENPGLWILG CHNSDFRNRG 720 |
| | MTALLKVSSC DKNTGDYYED SYEDISAYLL SKNNAIEPRS FSQNGTSESA TPESGPGSEP 780 |
| | ATSGSETPGT SESATPESGP GSEPATSGSE TPGTSESATP ESGPGTSTEP SEGSAPGSPA 840 |
| | GSPTSTEEGT SESATPESGP GSEPATSGSE TPGTSESATP ESGPGSPAGS PTSTEEGSPA 900 |
| | GSPTSTEEGT STEPSEGSAP GTSESATPES GPGTSESATP ESGPGTSESA TPESGPGSEP 960 |
| | ATSGSETPGS EPATSGSETP GSPAGSPIST EEGTSTEPSE GSAPGTSTEP SEGSAPGSEP 1020 |
| | ATSGSETPGT SESATPESGP GTSTEPSEGS APASSEITRT TLQSDQEEID YDDTISVEMK 1080 |
| | KEDFDIYDED ENQSPRSFQK KTRHYFIAAV ERLWDYGMSS SPHVLRNRAQ SGSVPQFKKV 1140 |
| | VFQEFTDGSF TQPLYRGELN EHLGLLGPYI RAEVEDNIMV TERNQASRPY SFYSSLISYE 1200 |
| | EDQRQGAEPR KNFVKPNETK TYFWKVQHHM APTKDEFDCK AWAYFSDVDL EKDVHSGLIG 1260 |
| | PLLVCHTNTL NPAHGRQVTV QEFALFFTIF DETKSWYFTE NMERNCRAPC NIQMEDPTFK 1320 |
| | ENYRFHAING YIMDTLPGLV MAQDQRIRWY LLSMGSNENI HSIHFSGHVF TVRKKEEYKM 1380 |
| | ALYNLYPGVF ETVEMLPSKA GIWRVECLIG EHLHAGMSTL FLVYSNKCQT PLGMASGHIR 1440 |
| | DFQITASGQY GQWAPKLARL HYSGSINAWS TKEPFSWIKV DLLAPMIIHG IKTQGARQKF 1500 |
| | SSLYISQFII MYSLDGKKWQ TYRGNSTGTL MVFFGNVDSS GIKHNIFNPP IIARYIRLHP 1560 |
| | THYSIRSTLR MELMGCDLNS CSMPLGMESK AISDAQITAS SYFTNMFATW SPSKARLHLQ 1620 |
| | GRSNAWRPQV NNPKEWLQVD FQKTMKVTGV TTQGVKSLLT SMYVKEFLIS SSQDGHQWTL 1680 |
| | FFQNGKVKVF QGNQDSFTPV VNSLDPPLLT RYLRIHPQSW VHQIALRMEV LGCEAQDLYD 1740 |
| | KTHTCPPCPA PELLGGPSVF LFPPKPKDTL MISRTPEVTC VVVDVSHEDP EVKFNWYVDG 1800 |
| | VEVHNAKTKP REEQYNSTYR VVSVLTVLHQ DWLNGKEYKC KVSNKALPAP IEKTISKAKG 1860 |
| | QPREPQVYTL PPSRDELTKN QVSLTCLVKG FYPSDIAVEW ESNGQPENNY KTTPPVLDSD 1920 |
| | GSFFLYSKLT VDKSRWQQGN VFSCSVMHEA LHNHYTQKSL SLSPG 1965 |
| rFVIIIFc-ELNN | atgcaaatag agctctccac ctgcttcttt ctgtgccttt tgcgattctg ctttagtgcc 60 |
| nucleotide | accagaagat actacctggg tgcagtggaa ctgtcatggg actatatgca aagtgatctc 120 |
| sequence | ggtgagctgc ctgtggacgc aagatttcct cctagagtgc caaaatcttt tccattcaac 180 |
| (SEQ ID NO: 2) | acctcagtcg tgtacaaaaa gactctgttt gtagaattca cggatcacct tttcaacatc 240 |
| | gctaagccaa ggccaccctg gatgggtctg ctaggtccta ccatccaggc tgaggtttat 300 |
| | gatacagtgg tcattacact taagaacatg gcttcccatc ctgtcagtct tcatgctgtt 360 |
| | ggtgtatcct actggaaagc ttctgaggga gctgaatatg atgatcagac cagtcaaagg 420 |
| | gagaaagaag atgataaagt cttccctggt ggaagccata catatgtctg gcaggtcctg 480 |
| | aaagagaatg gtccaatggc ctctgaccca ctgtgcctta cctactcata tctttctcat 540 |
| | gtggacctgg taaaagactt gaattcaggc ctcattggag ccctactagt atgtagagaa 600 |
| | gggagtctgg ccaaggaaaa gacacagacc ttgcacaaat ttatactact ttttgctgta 660 |
| | tttgatgaag ggaaaagttg gcactcagaa acaaagaact ccttgatgca ggatagggat 720 |
| | gctgcatctg ctcgggcctg gcctaaaatg cacacagtca atggttatgt aaacaggtct 780 |
| | ctgccaggtc tgattggatg ccacaggaaa tcagtctatt ggcatgtgat tggaatgggc 840 |
| | accactcctg aagtgcactc aatattcctc gaaggtcaca catttcttgt gaggaaccat 900 |
| | cgccaggcgt ccttggaaat ctcgccaata actttcctta ctgctcaaac actcttgatg 960 |
| | gaccttggac agtttctact gttttgtcat atctcttccc accaacatga tggcatggaa 1020 |
| | gcttatgtca aagtagacag ctgtccagag gaaccccaac tacgaatgaa aaataatgaa 1080 |
| | gaagcggaag actatgatga tgatcttact gattctgaaa tggatgtggt caggtttgat 1140 |
| | gatgacaact ctccttcctt tatccaaatt cgctcagttg ccaagaagca tcctaaaact 1200 |
| | tgggtacatt acattgctgc tgaagaggag gactgggact atgctccctt agtcctcgcc 1260 |
| | cccgatgaca gaagttataa aagtcaatat ttgaacaatg gccctcagcg gattggtagg 1320 |
| | aagtacaaaa aagtccgatt tatggcatac acagatgaaa cctttaagac tcgtgaagct 1380 |
| | attcagcatg aatcaggaat cttgggacct ttactttatg gggaagttgg agacacactg 1440 |
| | ttgattatat ttaagaatca agcaagcaga ccatataaca tctaccctca cggaatcact 1500 |
| | gatgtccgtc ctttgtattc aaggagatta ccaaaaggtg taaaacattt gaaggatttt 1560 |
| | ccaattctgc caggagaaat attcaaatat aaatggacag tgactgtaga agatgggcca 1620 |
| | actaaatcag atcctcggtg cctgacccgc tattactcta gtttcgttaa tatggagaga 1680 |
| | gatctagctt caggactcat tggccctctc ctcatctgct acaaagaatc tgtagatcaa 1740 |
| | agaggaaacc agataatgtc agacaagagg aatgtcatcc tgttttctgt atttgatgag 1800 |
| | aaccgaagct ggtacctcac agagaatata caacgctttc tccccaatcc agctggagtg 1860 |
| | cagcttgagg atccagagtt ccaagcctcc aacatcatgc acagcatcaa tggctatgtt 1920 |
| | tttgatagtt tgcagttgtc agtttgtttg catgaggtgg catactggta cattctaagc 1980 |
| | attggagcac agactgactt cctttctgtc ttcttctctg gatatacctt caaacacaaa 2040 |
| | atggtctatg aagacacact caccctattc ccattctcag gagaaactgt cttcatgtcg 2100 |
| | atggaaaacc caggtctatg gattctgggg tgccacaact cagactttcg gaacagaggc 2160 |
| | atgaccgcct tactgaaggt ttctagttgt gacaagaaca ctggtgatta ttacgaggac 2220 |
| | agttatgaag atatttcagc atacttgctg agtaaaaaca atgccattga accaagaagc 2280 |
| | ttctctcaaa acggtacctc agagtctgct acccccgagt cagggccagg atcagagcca 2340 |
| | gccacctccg ggtctgagac acccgggact tccgagagtg ccacccctga gtccggaccc 2400 |
| | gggtccgagc ccgccacttc cggctccgaa actcccggca caagcgagag cgctacccca 2460 |
| | gagtcaggac caggaacatc tacagagccc tctgaaggct ccgctccagg gtccccagcc 2520 |
| | ggcagtccca ctagcaccga ggagggaacc tctgaaagcg ccacacccga atcagggcca 2580 |
| | gggtctgagc ctgctaccag cggcagcgag acaccaggca cctctgagtc cgccacacca 2640 |
| | gagtccggac ccggatctcc cgctgggagc cccacctcca ctgaggaggg atctcctgct 2700 |
| | ggctctccaa catctactga ggaaggaacc tcaaccgagc catccgaggg atcagctccc 2760 |
| | ggcacctcag agtcggcaac cccggagtct ggacccggaa cttccgaaag tgccacacca 2820 |
| | gagtccggtc ccgggacttc agaatcagca acacccgagt ccggccctgg gtctgaaccc 2880 |
| | gccacaagtg gtagtgagac accaggatca gaacctgcta cctcagggtc agagacaccc 2940 |
| | ggatctccgg caggctcacc aacctccact gaggagggca ccagcacaga accaagcgag 3000 |
| | ggctccgcac ccggaacaag cactgaaccc agtgagggtt cagcacccgg ctctgagccg 3060 |
| | gccacaagtg gcagtgagac acccggcact tcagagagtg ccacccccga gagtggccca 3120 |
| | ggcactagta ccgagccctc tgaaggcagt gcgccagcct cgagcgaaat aactcgtact 3180 |
| | actcttcagt cagatcaaga ggaaattgac tatgatgata ccatatcagt tgaaatgaag 3240 |
| | aaggaagatt ttgacattta tgatgaggat gaaaatcaga gcccccgcag ctttcaaaag 3300 |
| | aaaacacgac actattttat tgctgcagtg gagaggctct gggattatgg gatgagtagc 3360 |
| | tccccacatg ttctaagaaa cagggctcag agtggcagtg tccctcagtt caagaaagtt 3420 |
| | gttttccagg aatttactga tggctccttt actcagccct tataccgtgg agaactaaat 3480 |
| | gaacatttgg gactcctggg gccatatata agagcagaag ttgaagataa tatcatggta 3540 |
| | actttcagaa atcaggcctc tcgtccctat tccttctatt ctagccttat ttcttatgag 3600 |
| | gaagatcaga ggcaaggagc agaacctaga aaaaactttg tcaagcctaa tgaaaccaaa 3660 |
| | acttactttt ggaaagtgca acatcatatg gcacccacta aagatgagtt tgactgcaaa 3720 |
| | gcctgggctt atttctctga tgttgacctg gaaaaagatg tgcactcagg cctgattgga 3780 |
| | ccccttctgg tctgccacac taacacactg aaccctgctc atgggagaca agtgacagta 3840 |
| | caggaatttg ctctgttttt caccatcttt gatgagacca aaagctggta cttcactgaa 3900 |
| | aatatggaaa gaaactgcag ggctccctgc aatatccaga tggaagatcc cacttttaaa 3960 |
| | gagaattatc gcttccatgc aatcaatggc tacataatgg atacactacc tggcttagta 4020 |
| | atggctcagg atcaaaggat tcgatggtat ctgctcagca tgggcagcaa tgaaaacatc 4080 |
| | cattctattc atttcagtgg acatgtgttc actgtacgaa aaaaagagga gtataaaatg 4140 |
| | gcactgtaca atctctatcc aggtgttttt gagacagtgg aaatgttacc atccaaagct 4200 |
| | ggaatttggc gggtggaatg ccttattggc gagcatctac atgctgggat gagcacactt 4260 |
| | tttctggtgt acagcaataa gtgtcagact cccctgggaa tggcttctgg acacattaga 4320 |
| | gattttcaga ttacagcttc aggacaatat ggacagtggg ccccaaagct ggccagactt 4380 |
| | cattattccg gatcaatcaa tgcctggagc accaaggagc ccttttcttg gatcaaggtg 4440 |
| | gatctgttgg caccaatgat tattcacggc atcaagaccc agggtgcccg tcagaagttc 4500 |
| | tccagcctct acatctctca gtttatcatc atgtatagtc ttgatgggaa gaagtggcag 4560 |
| | acttatcgag gaaattccac tggaacctta atggtcttct ttggcaatgt ggattcatct 4620 |
| | gggataaaac acaatatttt taaccctcca attattgctc gatacatccg tttgcaccca 4680 |
| | actcattata gcattcgcag cactcttcgc atggagttga tgggctgtga tttaaatagt 4740 |
| | tgcagcatgc cattgggaat ggagagtaaa gcaatatcag atgcacagat tactgcttca 4800 |
| | tcctacttta ccaatatgtt tgccacctgg tctccttcaa aagctcgact tcacctccaa 4860 |
| | gggaggagta atgcctggag acctcaggtg aataatccaa aagagtggct gcaagtggac 4920 |
| | ttccagaaga caatgaaagt cacaggagta actactcagg gagtaaaatc tctgcttacc 4980 |
| | agcatgtatg tgaaggagtt cctcatctcc agcagtcaag atggccatca gtggactctc 5040 |
| | ttttttcaga atggcaaagt aaaggttttt cagggaaatc aagactcctt cacacctgtg 5100 |
| | gtgaactctc tagacccacc gttactgact cgctaccttc gaattcaccc ccagagttgg 5160 |
| | gtgcaccaga ttgccctgag gatggaggtt ctgggctgcg aggcacagga cctctacgac 5220 |
| | aaaactcaca catgcccacc gtgcccagct ccagaactcc tgggcggacc gtcagtcttc 5280 |
| | ctcttccccc caaaacccaa ggacaccctc atgatctccc ggacccctga ggtcacatgc 5340 |
| | gtggtggtgg acgtgagcca cgaagaccct gaggtcaagt tcaactggta tgtggacggc 5400 |
| | gtggaagtgc ataatgccaa gacaaagccg cgggaggagc agtacaacag cacgtaccgt 5460 |
| | gtggtcagcg tcctcaccgt cctgcaccaa gactggctga atggcaagga gtacaagtgc 5520 |
| | aaggtctcca acaaagccct cccagccccc atcgagaaaa ccatctccaa agccaaaggg 5580 |
| | cagccccgag aaccacaggt gtacaccctg cccccatccc gggatgagct gaccaagaac 5640 |
| | caagttagcc tgacctgcct ggtcaaaggc ttctatccca gcgacatcgc cgtggagtgg 5700 |
| | gagagcaatg ggcagccgga gaacaactac aagaccacgc ctcccgtgtt ggactccgac 5760 |
| | ggctccttct tcctctactc caagctcacc gtggacaaga gcaggtggca gcaggggaac 5820 |
| | gtcttctcat gctccgtgat gcatgaggct ctgcacaacc actacacgca gaagagcctc 5880 |
| | tccctgtctc cgggttga 5898 |
| rFVIIIFc-ELNN | ATRRYYLGAV ELSWDYMQSD LGELPVDARF PPRVPKSFPF NTSVVYKKTL FVEFTDHLEN 60 |
| amino acid | IAKPRPPWMG LLGPTIQAEV YDTVVITLKN MASHPVSLHA VGVSYWKASE GAEYDDQTSQ 120 |
| sequence, | REKEDDKVFP GGSHTYVWQV LKENGPMASD PLCLTYSYLS HVDLVKDLNS GLIGALLVCR 180 |
| without signal | EGSLAKEKTQ TLHKFILLFA VFDEGKSWHS ETKNSLMQDR DAASARAWPK MHTVNGYVNR 240 |
| peptide | SLPGLIGCHR KSVYWHVIGM GTTPEVHSIF LEGHTFLVRN HRQASLEISP ITFLTAQTLL 300 |
| (SEQ ID NO: 3) | MDLGQFLLFC HISSHQHDGM EAYVKVDSCP EEPQLRMKNN EEAEDYDDDL TDSEMDVVRF 360 |
| | DDDNSPSFIQ IRSVAKKHPK TWVHYIAAEE EDWDYAPLVL APDDRSYKSQ YLNNGPQRIG 420 |
| | RKYKKVRFMA YTDETFKTRE AIQHESGILG PLLYGEVGDT LLIIFKNQAS RPYNIYPHGI 480 |
| | TDVRPLYSRR LPKGVKHLKD FPILPGEIFK YKWTVTVEDG PTKSDPRCLT RYYSSFVNME 540 |
| | RDLASGLIGP LLICYKESVD QRGNQIMSDK RNVILFSVED ENRSWYLTEN IQRFLPNPAG 600 |
| | VQLEDPEFQA SNIMHSINGY VFDSLQLSVC LHEVAYWYIL SIGAQTDFLS VFFSGYTFKH 660 |
| | KMVYEDTLTL FPFSGETVFM SMENPGLWIL GCHNSDFRNR GMTALLKVSS CDKNTGDYYE 720 |
| | DSYEDISAYL LSKNNAIEPR SFSQNGTSES ATPESGPGSE PATSGSETPG TSESATPESG 780 |
| | PGSEPATSGS ETPGTSESAT PESGPGTSTE PSEGSAPGSP AGSPTSTEEG TSESATPESG 840 |
| | PGSEPATSGS ETPGTSESAT PESGPGSPAG SPTSTEEGSP AGSPTSTEEG TSTEPSEGSA 900 |
| | PGTSESATPE SGPGTSESAT PESGPGTSES ATPESGPGSE PATSGSETPG SEPATSGSET 960 |
| | PGSPAGSPTS TEEGTSTEPS EGSAPGTSTE PSEGSAPGSE PATSGSETPG TSESATPESG 1020 |
| | PGTSTEPSEG SAPASSEITR TTLQSDQEEI DYDDTISVEM KKEDEDIYDE DENQSPRSFQ 1080 |
| | KKTRHYFIAA VERLWDYGMS SSPHVLRNRA QSGSVPQFKK VVFQEFTDGS FTQPLYRGEL 1140 |
| | NEHLGLLGPY IRAEVEDNIM VTERNQASRP YSFYSSLISY EEDQRQGAEP RKNFVKPNET 1200 |
| | KTYFWKVQHH MAPTKDEFDC KAWAYFSDVD LEKDVHSGLI GPLLVCHTNT LNPAHGRQVT 1260 |
| | VQEFALFFTI FDETKSWYFT ENMERNCRAP CNIQMEDPTF KENYRFHAIN GYIMDTLPGL 1320 |
| | VMAQDQRIRW YLLSMGSNEN IHSIHFSGHV FTVRKKEEYK MALYNLYPGV FETVEMLPSK 1380 |
| | AGIWRVECLI GEHLHAGMST LFLVYSNKCQ TPLGMASGHI RDFQITASGQ YGQWAPKLAR 1440 |
| | LHYSGSINAW STKEPFSWIK VDLLAPMIIH GIKTQGARQK FSSLYISQFI IMYSLDGKKW 1500 |
| | QTYRGNSTGT LMVFFGNVDS SGIKHNIFNP PIIARYIRLH PTHYSIRSTL RMELMGCDLN 1560 |
| | SCSMPLGMES KAISDAQITA SSYFTNMFAT WSPSKARLHL QGRSNAWRPQ VNNPKEWLQV 1620 |
| | DFQKTMKVTG VTTQGVKSLL TSMYVKEFLI SSSQDGHQWT LFFQNGKVKV FQGNQDSFTP 1680 |
| | VVNSLDPPLL TRYLRIHPQS WVHQIALRME VLGCEAQDLY DKTHTCPPCP APELLGGPSV 1740 |
| | FLFPPKPKDT LMISRTPEVT CVVVDVSHED PEVKFNWYVD GVEVHNAKTK PREEQYNSTY 1800 |
| | RVVSVLTVLH QDWLNGKEYK CKVSNKALPA PIEKTISKAK GQPREPQVYT LPPSRDELTK 1860 |
| | NQVSLTCLVK GFYPSDIAVE WESNGQPENN YKTTPPVLDS DGSFFLYSKL TVDKSRWQQG 1920 |
| | NVFSCSVMHE ALHNHYTQKS LSLSPG 1946 |
| rVWFFc-ELNN | MIPARFAGVL LALALILPGT LCAEGTRGRS STARCSLFGS DFVNTFDGSM YSFAGYCSYL 60 |
| amino acid | LAGGCQKRSF SIIGDFQNGK RVSLSVYLGE FFDIHLFVNG TVTQGDQRVS MPYASKGLYL 120 |
| sequence, | ETEAGYYKLS GEAYGFVARI DGSGNFQVLL SDRYFNKTCG LCGNFNIFAE DDFMTQEGTL 180 |
| with signal | TSDPYDFANS WALSSGEQWC ERASPPSSSC NISSGEMQKG LWEQCQLLKS TSVFARCHPL 240 |
| peptide and | VDPEPFVALC EKTLCECAGG LECACPALLE YARTCAQEGM VLYGWTDHSA CSPVCPAGME 300 |
| D1D2 portion | YRQCVSPCAR TCQSLHINEM CQERCVDGCS CPEGQLLDEG LCVESTECPC VHSGKRYPPG 360 |
| of VWF | TSLSRDCNTC ICRNSQWICS NEECPGECLV TGQSHFKSED NRYFTFSGIC QYLLARDCQD 420 |
| (SEQ ID NO: 4) | HSFSIVIETV QCADDRDAVC TRSVTVRLPG LHNSLVKLKH GAGVAMDGQD IQLPLLKGDL 480 |
| | RIQHTVTASV RLSYGEDLQM DWDGRGRLLV KLSPVYAGKT CGLCGNYNGN QGDDFLTPSG 540 |
| | LAEPRVEDFG NAWKLHGDCQ DLQKQHSDPC ALNPRMTRES EEACAVLTSP TFEACHRAVS 600 |
| | PLPYLRNCRY DVCSCSDGRE CLCGALASYA AACAGRGVRV AWREPGRCEL NCPKGQVYLQ 660 |
| | CGTPCNLTCR SLSYPDEECN EACLEGCFCP PGLYMDERGD CVPKAQCPCY YDGEIFQPED 720 |
| | IFSDHHTMCY CEDGFMHCTM SGVPGSLLPD AVLSSPLSHR SKRSLSCRPP MVKLVCPADN 780 |
| | LRAEGLECTK TCQNYDLECM SMGCVSGCLC PPGMVRHENR CVALERCPCF HQGKEYAPGE 840 |
| | TVKIGCNTCV CRDRKWNCTD HVCDATCSTI GMAHYLTEDG LKYLFPGECQ YVLVQDYCGS 900 |
| | NPGTFRILVG NKGCSHPSVK CKKRVTILVE GGEIELFDGE VNVKRPMKDE THFEVVESGR 960 |
| | YIILLLGKAL SVVWDRHLSI SVVLKQTYQE KVCGLCGNED GIQNNDLTSS NLQVEEDPVD 1020 |
| | FGNSWKVSSQ CADTRKVPLD SSPATCHNNI MKQTMVDSSC RILTSDVFQD CNKLVDPEPY 1080 |
| | LDVCIYDTCS CESIGDCAAF CDTIAAYAHV CAQHGKVVTW RTATLCPQSC EERNLRENGY 1140 |
| | EAEWRYNSCA PACQVTCQHP EPLACPVQCV EGCHAHCPPG KILDELLQTC VDPEDCPVCE 1200 |
| | VAGRRFASGK KVTLNPSDPE HCQICHCDVV NLTCEACQEP GTSESATPES GPGSEPATSG 1260 |
| | SETPGTSESA TPESGPGSEP ATSGSETPGT SESATPESGP GTSTEPSEGS APGSPAGSPT 1320 |
| | STEEGTSESA TPESGPGSEP ATSGSETPGT SESATPESGP GSPAGSPTST EEGSPAGSPT 1380 |
| | STEEGASSDK NIGDYYEDSY EDISAYLLSK NNAIEPRSFS DKTHTCPPCP APELLGGPSV 1440 |
| | FLFPPKPKDT LMISRTPEVT CVVVDVSHED PEVKENWYVD GVEVHNAKTK PREEQYNSTY 1500 |
| | RVVSVLTVLH QDWLNGKEYK CKVSNKALPA PIEKTISKAK GQPREPQVYT LPPSRDELTK 1560 |
| | NQVSLTCLVK GFYPSDIAVE WESNGQPENN YKTTPPVLDS DGSFFLYSKL TVDKSRWQQG 1620 |
| | NVFSCSVMHE ALHNHYTQKS LSLSPG 1646 |
| rVWFFc-ELNN | atgattcctg ccagatttgc cggggtgctg cttgctctgg ccctcatttt gccagggacc 60 |
| nucleotide | ctttgtgcag aaggaactcg cggcaggtca tccacggccc gatgcagcct tttcggaagt 120 |
| sequence | gacttcgtca acacctttga tgggagcatg tacagctttg cgggatactg cagttacctc 180 |
| (SEQ ID NO: 5) | ctggcagggg gctgccagaa acgctccttc tcgattattg gggacttcca gaatggcaag 240 |
| | agagtgagcc tctccgtgta tcttggggaa ttttttgaca tccatttgtt tgtcaatggt 300 |
| | accgtgacac agggggacca aagagtctcc atgccctatg cctccaaagg gctgtatcta 360 |
| | gaaactgagg ctgggtacta caagctgtcc ggtgaggcct atggctttgt ggccaggatc 420 |
| | gatggcagcg gcaactttca agtcctgctg tcagacagat acttcaacaa gacctgcggg 480 |
| | ctgtgtggca actttaacat ctttgctgaa gatgacttta tgacccaaga agggaccttg 540 |
| | acctcggacc cttatgactt tgccaactca tgggctctga gcagtggaga acagtggtgt 600 |
| | gaacgggcat ctcctcccag cagctcatgc aacatctcct ctggggaaat gcagaagggc 660 |
| | ctgtgggagc agtgccagct tctgaagagc acctcggtgt ttgcccgctg ccaccctctg 720 |
| | gtggaccccg agccttttgt ggccctgtgt gagaagactt tgtgtgagtg tgctgggggg 780 |
| | ctggagtgcg cctgccctgc cctcctggag tacgcccgga cctgtgccca ggagggaatg 840 |
| | gtgctgtacg gctggaccga ccacagcgcg tgcagcccag tgtgccctgc tggtatggag 900 |
| | tataggcagt gtgtgtcccc ttgcgccagg acctgccaga gcctgcacat caatgaaatg 960 |
| | tgtcaggagc gatgcgtgga tggctgcagc tgccctgagg gacagctcct ggatgaaggc 1020 |
| | ctctgcgtgg agagcaccga gtgtccctgc gtgcattccg gaaagcgcta ccctcccggc 1080 |
| | acctccctct ctcgagactg caacacctgc atttgccgaa acagccagtg gatctgcagc 1140 |
| | aatgaagaat gtccagggga gtgccttgtc actggtcaat cccacttcaa gagctttgac 1200 |
| | aacagatact tcaccttcag tgggatctgc cagtacctgc tggcccggga ttgccaggac 1260 |
| | cactccttct ccattgtcat tgagactgtc cagtgtgctg atgaccgcga cgctgtgtgc 1320 |
| | acccgctccg tcaccgtccg gctgcctggc ctgcacaaca gccttgtgaa actgaagcat 1380 |
| | ggggcaggag ttgccatgga tggccaggac atccagctcc ccctcctgaa aggtgacctc 1440 |
| | cgcatccagc atacagtgac ggcctccgtg cgcctcagct acggggagga cctgcagatg 1500 |
| | gactgggatg gccgcgggag gctgctggtg aagctgtccc ccgtctatgc cgggaagacc 1560 |
| | tgcggcctgt gtgggaatta caatggcaac cagggcgacg acttccttac cccctctggg 1620 |
| | ctggcggagc cccgggtgga ggacttcggg aacgcctgga agctgcacgg ggactgccag 1680 |
| | gacctgcaga agcagcacag cgatccctgc gccctcaacc cgcgcatgac caggttctcc 1740 |
| | gaggaggcgt gcgcggtcct gacgtccccc acattcgagg cctgccatcg tgccgtcagc 1800 |
| | ccgctgccct acctgcggaa ctgccgctac gacgtgtgct cctgctcgga cggccgcgag 1860 |
| | tgcctgtgcg gcgccctggc cagctatgcc gcggcctgcg cggggagagg cgtgcgcgtc 1920 |
| | gcgtggcgcg agccaggccg ctgtgagctg aactgcccga aaggccaggt gtacctgcag 1980 |
| | tgcgggaccc cctgcaacct gacctgccgc tctctctctt acccggatga ggaatgcaat 2040 |
| | gaggcctgcc tggagggctg cttctgcccc ccagggctct acatggatga gaggggggac 2100 |
| | tgcgtgccca aggcccagtg cccctgttac tatgacggtg agatcttcca gccagaagac 2160 |
| | atcttctcag accatcacac catgtgctac tgtgaggatg gcttcatgca ctgtaccatg 2220 |
| | agtggagtcc ccggaagctt gctgcctgac gctgtcctca gcagtcccct gtctcatcgc 2280 |
| | agcaaaagga gcctatcctg tcggcccccc atggtcaagc tggtgtgtcc cgctgacaac 2340 |
| | ctgcgggctg aagggctcga gtgtaccaaa acgtgccaga actatgacct ggagtgcatg 2400 |
| | agcatgggct gtgtctctgg ctgcctctgc cccccgggca tggtccggca tgagaacaga 2460 |
| | tgtgtggccc tggaaaggtg tccctgcttc catcagggca aggagtatgc ccctggagaa 2520 |
| | acagtgaaga ttggctgcaa cacttgtgtc tgtcgggacc ggaagtggaa ctgcacagac 2580 |
| | catgtgtgtg atgccacgtg ctccacgatc ggcatggccc actacctcac cttcgacggg 2640 |
| | ctcaaatacc tgttccccgg ggagtgccag tacgttctgg tgcaggatta ctgcggcagt 2700 |
| | aaccctggga cctttcggat cctagtgggg aataagggat gcagccaccc ctcagtgaaa 2760 |
| | tgcaagaaac gggtcaccat cctggtggag ggaggagaga ttgagctgtt tgacggggag 2820 |
| | gtgaatgtga agaggcccat gaaggatgag actcactttg aggtggtgga gtctggccgg 2880 |
| | tacatcattc tgctgctggg caaagccctc tccgtggtct gggaccgcca cctgagcatc 2940 |
| | tccgtggtcc tgaagcagac ataccaggag aaagtgtgtg gcctgtgtgg gaattttgat 3000 |
| | ggcatccaga acaatgacct caccagcagc aacctccaag tggaggaaga ccctgtggac 3060 |
| | tttgggaact cctggaaagt gagctcgcag tgtgctgaca ccagaaaagt gcctctggac 3120 |
| | tcatcccctg ccacctgcca taacaacatc atgaagcaga cgatggtgga ttcctcctgt 3180 |
| | agaatcctta ccagtgacgt cttccaggac tgcaacaagc tggtggaccc cgagccatat 3240 |
| | ctggatgtct gcatttacga cacctgctcc tgtgagtcca ttggggactg cgccgcattc 3300 |
| | tgcgacacca ttgctgccta tgcccacgtg tgtgcccagc atggcaaggt ggtgacctgg 3360 |
| | aggacggcca cattgtgccc ccagagctgc gaggagagga atctccggga gaacgggtat 3420 |
| | gaggctgagt ggcgctataa cagctgtgca cctgcctgtc aagtcacgtg tcagcaccct 3480 |
| | gagccactgg cctgccctgt gcagtgtgtg gagggctgcc atgcccactg ccctccaggg 3540 |
| | aaaatcctgg atgagctttt gcagacctgc gttgaccctg aagactgtcc agtgtgtgag 3600 |
| | gtggctggcc ggcgttttgc ctcaggaaag aaagtcacct tgaatcccag tgaccctgag 3660 |
| | cactgccaga tttgccactg tgatgttgtc aacctcacct gtgaagcctg ccaggagccg 3720 |
| | ggtacatcag agagcgccac ccctgaaagt ggtcccggga gcgagccagc cacatctggg 3780 |
| | tcggaaacgc caggcacatc cgagtctgca actcccgagt ccggacctgg ctccgagcct 3840 |
| | gccactagcg gctccgagac tccgggaact tccgagagcg ctacaccaga aagcggaccc 3900 |
| | ggaaccagta ccgaacctag cgagggctct gctccgggca gcccagccgg ctctcctaca 3960 |
| | tccacggagg agggcacttc cgaatccgcc accccggagt cagggccagg atctgaaccc 4020 |
| | gctacctcag gcagtgagac gccaggaacg agcgagtccg ctacaccgga gagtgggcca 4080 |
| | gggagccctg ctggatctcc tacgtccact gaggaagggt caccagcggg ctcgcccacc 4140 |
| | agcactgaag aaggtgcctc gtctgacaag aacactggtg attattacga ggacagttat 4200 |
| | gaagatattt cagcatactt gctgagtaaa aacaatgcca ttgaaccaag aagcttctct 4260 |
| | gacaaaactc acacatgccc accgtgccca gctccagaac tcctgggcgg accgtcagtc 4320 |
| | ttcctcttcc ccccaaaacc caaggacacc ctcatgatct cccggacccc tgaggtcaca 4380 |
| | tgcgtggtgg tggacgtgag ccacgaagac cctgaggtca agttcaactg gtatgtggac 4440 |
| | ggcgtggaag tgcataatgc caagacaaag ccgcgggagg agcagtacaa cagcacgtac 4500 |
| | cgtgtggtca gcgtcctcac cgtcctgcac caagactggc tgaatggcaa ggagtacaag 4560 |
| | tgcaaggtct ccaacaaagc cctcccagcc cccatcgaga aaaccatctc caaagccaaa 4620 |
| | gggcagcccc gagaaccaca ggtgtacacc ctgcccccat cccgggatga gctgaccaag 4680 |
| | aaccaagtta gcctgacctg cctggtcaaa ggcttctatc ccagcgacat cgccgtggag 4740 |
| | tgggagagca atgggcagcc ggagaacaac tacaagacca cgcctcccgt gttggactcc 4800 |
| | gacggctcct tcttcctcta ctccaagctc accgtggaca agagcaggtg gcagcagggg 4860 |
| | aacgtcttct catgctccgt gatgcatgag gctctgcaca accactacac gcagaagagc 4920 |
| | ctctccctgt ctccgggttg a 4941 |
| rVWF-ELNN- | SLSCRPPMVK LVCPADNLRA EGLECTKTCQ NYDLECMSMG CVSGCLCPPG MVRHENRCVA 60 |
| Fc | LERCPCFHQG KEYAPGETVK IGCNTCVCRD RKWNCTDHVC DATCSTIGMA HYLTFDGLKY 120 |
| amino acid | LFPGECQYVL VQDYCGSNPG TFRILVGNKG CSHPSVKCKK RVTILVEGGE IELFDGEVNV 180 |
| sequence, | KRPMKDETHF EVVESGRYII LLLGKALSVV WDRHLSISVV LKQTYQEKVC GLCGNFDGIQ 240 |
| without signal | NNDLTSSNLQ VEEDPVDFGN SWKVSSQCAD TRKVPLDSSP ATCHNNIMKQ TMVDSSCRIL 300 |
| peptide or | TSDVFQDCNK LVDPEPYLDV CIYDTCSCES IGDCAAFCDT IAAYAHVCAQ HGKVVTWRTA 360 |
| D1D2 portion | TLCPQSCEER NLRENGYEAE WRYNSCAPAC QVTCQHPEPL ACPVQCVEGC HAHCPPGKIL 420 |
| of VWF | DELLQTCVDP EDCPVCEVAG RRFASGKKVT LNPSDPEHCQ ICHCDVVNLT CEACQEPGTS 480 |
| (SEQ ID NO: 6) | ESATPESGPG SEPATSGSET PGTSESATPE SGPGSEPATS GSETPGTSES ATPESGPGTS 540 |
| | TEPSEGSAPG SPAGSPTSTE EGTSESATPE SGPGSEPATS GSETPGTSES ATPESGPGSP 600 |
| | AGSPTSTEEG SPAGSPTSTE EGASSDKNTG DYYEDSYEDI SAYLLSKNNA IEPRSFSDKT 660 |
| | HTCPPCPAPE LLGGPSVFLF PPKPKDTLMI SRTPEVTCVV VDVSHEDPEV KFNWYVDGVE 720 |
| | VHNAKTKPRE EQYNSTYRVV SVLTVLHQDW LNGKEYKCKV SNKALPAPIE KTISKAKGQP 780 |
| | REPQVYTLPP SRDELTKNQV SLTCLVKGFY PSDIAVEWES NGQPENNYKT TPPVLDSDGS 840 |
| | FFLYSKLTVD KSRWQQGNVF SCSVMHEALH NHYTQKSLSL SPG 883 |
| Mature human | ATRRYYLGAV ELSWDYMQSD LGELPVDARF PPRVPKSFPF NTSVVYKKTL FVEFTDHLEN 60 |
| FVIII | IAKPRPPWMG LLGPTIQAEV YDTVVITLKN MASHPVSLHA VGVSYWKASE GAEYDDQTSQ 120 |
| (SEQ ID NO: 7) | REKEDDKVFP GGSHTYVWQV LKENGPMASD PLCLTYSYLS HVDLVKDLNS GLIGALLVCR 180 |
| | EGSLAKEKTQ TLHKFILLFA VFDEGKSWHS ETKNSLMQDR DAASARAWPK MHTVNGYVNR 240 |
| | SLPGLIGCHR KSVYWHVIGM GTTPEVHSIF LEGHTFLVRN HRQASLEISP ITFLTAQTLL 300 |
| | MDLGQFLLFC HISSHQHDGM EAYVKVDSCP EEPQLRMKNN EEAEDYDDDL TDSEMDVVRF 360 |
| | DDDNSPSFIQ IRSVAKKHPK TWVHYIAAEE EDWDYAPLVL APDDRSYKSQ YLNNGPQRIG 420 |
| | RKYKKVRFMA YTDETFKTRE AIQHESGILG PLLYGEVGDT LLIIFKNQAS RPYNIYPHGI 480 |
| | TDVRPLYSRR LPKGVKHLKD FPILPGEIFK YKWTVTVEDG PTKSDPRCLT RYYSSFVNME 540 |
| | RDLASGLIGP LLICYKESVD QRGNQIMSDK RNVILFSVFD ENRSWYLTEN IQRFLPNPAG 600 |
| | VQLEDPEFQA SNIMHSINGY VFDSLQLSVC LHEVAYWYIL SIGAQTDFLS VFFSGYTFKH 660 |
| | KMVYEDTLTL FPFSGETVFM SMENPGLWIL GCHNSDFRNR GMTALLKVSS CDKNTGDYYE 720 |
| | DSYEDISAYL LSKNNAIEPR SFSQNSRHPS TRQKQFNATT IPENDIEKTD PWFAHRTPMP 780 |
| | KIQNVSSSDL LMLLRQSPTP HGLSLSDLQE AKYETFSDDP SPGAIDSNNS LSEMTHFRPQ 840 |
| | LHHSGDMVFT PESGLQLRLN EKLGTTAATE LKKLDFKVSS ISNNLISTIP SDNLAAGTDN 900 |
| | TSSLGPPSMP VHYDSQLDTT LFGKKSSPLT ESGGPLSLSE ENNDSKLLES GLMNSQESSW 960 |
| | GKNVSSTESG RLFKGKRAHG PALLTKDNAL FKVSISLLKT NKTSNNSATN RKTHIDGPSL 1020 |
| | LIENSPSVWQ NILESDTEFK KVTPLIHDRM LMDKNATALR LNHMSNKTTS SKNMEMVQQK 1080 |
| | KEGPIPPDAQ NPDMSFFKML FLPESARWIQ RTHGKNSLNS GQGPSPKQLV SLGPEKSVEG 1140 |
| | QNFLSEKNKV VVGKGEFTKD VGLKEMVFPS SRNLFLINLD NLHENNTHNQ EKKIQEEIEK 1200 |
| | KETLIQENVV LPQIHTVTGT KNFMKNLFLL STRQNVEGSY DGAYAPVLQD FRSLNDSTNR 1260 |
| | TKKHTAHFSK KGEEENLEGL GNQTKQIVEK YACTTRISPN TSQQNFVTQR SKRALKQFRL 1320 |
| | PLEETELEKR IIVDDTSTQW SKNMKHLTPS TLTQIDYNEK EKGAITQSPL SDCLTRSHSI 1380 |
| | PQANRSPLPI AKVSSFPSIR PIYLTRVLFQ DNSSHLPAAS YRKKDSGVQE SSHFLQGAKK 1440 |
| | NNLSLAILTL EMTGDQREVG SLGTSATNSV TYKKVENTVL PKPDLPKTSG KVELLPKVHI 1500 |
| | YQKDLFPTET SNGSPGHLDL VEGSLLQGTE GAIKWNEANR PGKVPFLRVA TESSAKTPSK 1560 |
| | LLDPLAWDNH YGTQIPKEEW KSQEKSPEKT AFKKKDTILS LNACESNHAI AAINEGQNKP 1620 |
| | EIEVTWAKQG RTERLCSQNP PVLKRHQREI TRTTLQSDQE EIDYDDTISV EMKKEDEDIY 1680 |
| | DEDENQSPRS FQKKTRHYFI AAVERLWDYG MSSSPHVLRN RAQSGSVPQF KKVVFQEFTD 1740 |
| | GSFTQPLYRG ELNEHLGLLG PYIRAEVEDN IMVTERNQAS RPYSFYSSLI SYEEDQRQGA 1800 |
| | EPRKNFVKPN ETKTYFWKVQ HHMAPTKDEF DCKAWAYFSD VDLEKDVHSG LIGPLLVCHT 1860 |
| | NTLNPAHGRQ VTVQEFALFF TIFDETKSWY FTENMERNCR APCNIQMEDP TFKENYRFHA 1920 |
| | INGYIMDTLP GLVMAQDQRI RWYLLSMGSN ENIHSIHFSG HVFTVRKKEE YKMALYNLYP 1980 |
| | GVFETVEMLP SKAGIWRVEC LIGEHLHAGM STLFLVYSNK CQTPLGMASG HIRDFQITAS 2040 |
| | GQYGQWAPKL ARLHYSGSIN AWSTKEPFSW IKVDLLAPMI IHGIKTQGAR QKFSSLYISQ 2100 |
| | FIIMYSLDGK KWQTYRGNST GTLMVFFGNV DSSGIKHNIF NPPIIARYIR LHPTHYSIRS 2160 |
| | TLRMELMGCD LNSCSMPLGM ESKAISDAQI TASSYFTNMF ATWSPSKARL HLQGRSNAWR 2220 |
| | PQVNNPKEWL QVDFQKTMKV TGVTTQGVKS LLTSMYVKEF LISSSQDGHQ WTLFFQNGKV 2280 |
| | KVFQGNQDSF TPVVNSLDPP LLTRYLRIHP QSWVHQIALR MEVLGCEAQD LY 2332 |
| ELNN | GTSESATPES GPGSEPATSG SETPGTSESA TPESGPGSEP ATSGSETPGT SESATPESGP 60 |
| polypeptide | GTSTEPSEGS APGSPAGSPT STEEGTSESA TPESGPGSEP ATSGSETPGT SESATPESGP 120 |
| AE288 | GSPAGSPTST EEGSPAGSPT STEEGTSTEP SEGSAPGTSE SATPESGPGT SESATPESGP 180 |
| (SEQ ID NO: 8) | GTSESATPES GPGSEPATSG SETPGSEPAT SGSETPGSPA GSPTSTEEGT STEPSEGSAP 240 |
| | GTSTEPSEGS APGSEPATSG SETPGTSESA TPESGPGTST EPSEGSAP 288 |
| ELNN | TSESATPESG PGSEPATSGS ETPGTSESAT PESGPGSEPA TSGSETPGTS ESATPESGPG 60 |
| polypeptide | TSTEPSEGSA PGSPAGSPTS TEEGTSESAT PESGPGSEPA TSGSETPGTS ESATPESGPG 120 |
| AE144_5A | SPAGSPTSTE EGSPAGSPTS TEEG 144 |
| (SEQ ID NO: 9) | |
| a2 Linker of | DKNTGDYYED SYEDI SAYLL SKNNAIEPRS FS 32 |
| chimeric | |
| protein | |
| (SEQ ID NO: | |
| 10) | |

1. A method of estimating bleed risk, the method comprising:
(a) receiving clotting Factor VIII (FVIII) activity information by a software-based system comprising a computer program that is programmed to implement a Repeated Time to Event (RTTE) model,
(b) calculating, by the computer program, bleed risk using the RTTE model and the received information, and
(c) transmitting, by the software-based system, the calculated bleed risk information of (b) for output of the bleed risk information.

2. (canceled)

3. The method of claim 1, wherein calculating bleed risk is estimating the risk of occurrence of a bleeding event.

4. (canceled)

5. (canceled)

6. The method of claim 1, wherein the FVIII activity information is provided by an efanesoctocog alfa popPK model comprising body weight as a covariate, and wherein the efanesoctocog alfa popPK model does not comprise the level of VWF or level of hematocrit as covariates.

7. The method of claim 6, wherein the popPK model is an efanesoctocog alfa popPK model [A] or [A′].

8. (canceled)

9. The method of claim 1, wherein the FVIII activity information provided is for an individual subject, a population of subjects, or a virtual or hypothetical subject.

10. The method of claim 1, wherein the FVIII activity information provided is a target FVIII activity level.

11-18. (canceled)

19. The method of claim 1, wherein the RTTE model comprises treatment effect as a covariate.

20. (canceled)

21. The method of claim 19, wherein the RTTE model is the RTTE model [B], [C], [D], [D1], or [D2].

22-27. (canceled)

28. The method of claim 10, wherein the desired treatment outcome information is provided by the individual subject or a healthcare professional.

29. (canceled)

30. A method of treating a subject with hemophila A, the method comprising:
(a) identifying a subject with hemophilia A;
(b) estimating the bleed risk in the subject when treated with 50 IU/kg efanesoctocog alfa about once weekly according to the method of claim 1,
(c) estimating the bleed risk in the subject if the subject had FVIII activity level of at least 10 IU/dL according to the method of claim 1; and
(d) treating the subject with 50 IU/kg efanesoctocog alfa about once weekly if the subject has a lower estimated bleed risk with the efanesoctocog alfa therapy than the estimated bleed risk if the subject had a FVIII activity level of at least 10 IU/dL.

31. (canceled)

32. (canceled)

33. The method of claim 1, wherein the subject is at least 12 years old.

34. (canceled)

35. The method of any one of claim 1, wherein the wherein the subject is less than 12 years old.

36. (canceled)

37. (canceled)

38. A data processing apparatus, device, or system comprising a processor configured to implement an efanesoctocog alfa RTTE model.

39-48. (canceled)

49. A computer-readable medium comprising instructions which, when executed by a computer, cause the computer to carry out the method of claim 1.

50. (canceled)

51. (canceled)


SEQUENCE LISTING (10 sequences)

SEQ ID NO: 1 - protein, 1965 aa, synthetic construct
  REGION 1..1965: note = rFVIIIFc-XTEN amino acid sequence, including signal peptide
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - DNA, 5898 bp, synthetic construct
  misc_feature 1..5898: note = rFVIIIFc-XTEN nucleotide sequence
  source 1..5898: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggaacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccagcctcgagcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaagtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaagttagcctgacctgcctggtcaaaggcttctatcccagcgacatcgccgtggagtgggagagcaatgggcagccggagaacaactacaagaccacgcctcccgtgttggactccgacggctccttcttcctctactccaagctcaccgtggacaagagcaggtggcagcaggggaacgtcttctcatgctccgtgatgcatgaggctctgcacaaccactacacgcagaagagcctctccctgtctccgggttga

SEQ ID NO: 3 - protein, 1946 aa, synthetic construct
  REGION 1..1946: note = rFVIIIFc-XTEN amino acid sequence without signal peptide
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 1646 aa, synthetic construct
  REGION 1..1646: note = rVWFFc-XTEN amino acid sequence with signal peptide and D1D2portion of VWF
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - DNA, 4941 bp, synthetic construct
  misc_feature 1..4941: note = rVWFFc-XTEN nucleotide sequence
  source 1..4941: mol_type = other DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggtacatcagagagcgccacccctgaaagtggtcccgggagcgagccagccacatctgggtcggaaacgccaggcacatccgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgtctgacaagaacactggtgattattacgaggacagttatgaagatatttcagcatacttgctgagtaaaaacaatgccattgaaccaagaagcttctctgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaagtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaagttagcctgacctgcctggtcaaaggcttctatcccagcgacatcgccgtggagtgggagagcaatgggcagccggagaacaactacaagaccacgcctcccgtgttggactccgacggctccttcttcctctactccaagctcaccgtggacaagagcaggtggcagcaggggaacgtcttctcatgctccgtgatgcatgaggctctgcacaaccactacacgcagaagagcctctccctgtctccgggttga

SEQ ID NO: 6 - protein, 883 aa, synthetic construct
  REGION 1..883: note = rVWFFc-XTEN amino acid sequence without signal peptide or D1D2portion of VWF
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 8 - protein, 288 aa, synthetic construct
  REGION 1..288: note = Synthesized
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 9 - protein, 144 aa, synthetic construct
  REGION 1..144: note = Synthesized
TSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEG

SEQ ID NO: 10 - protein, 32 aa, synthetic construct
  REGION 1..32: note = Synthesized
DKNTGDYYEDSYEDISAYLLSKNNAIEPRSFS